SEQUENCE LISTING (104 sequences)

SEQ ID NO: 1 - protein, 121 aa, Mus musculus
QVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSS

SEQ ID NO: 2 - protein, 107 aa, Mus musculus
DIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIK

SEQ ID NO: 3 - protein, 121 aa, Artificial
  A03 CAR VH
QVQLQESGPGLVRPSQTLSLTCTVSGFTFTSYGVSWVRQPPGRGLEWIGVIWEDGSTNYHSALISRVTMLVDTSKNQFSLRLSSVTAADTAVYYCARPHYGSSYVGAMEYWGQGSLVTVSS

SEQ ID NO: 4 - protein, 108 aa, Artificial
  A03 CAR VL
DIQMTQSPSSLSASVGDRVTITCRASENIYSYVAWYQQKPGKAPKLLIYNAKSLAEGVPSRFSGSGSGTDFTFTISSLQPEDIATYYCQHHYVSPWTFGQGTKVEIKR

SEQ ID NO: 5 - protein, 121 aa, Artificial
  A04 CAR VH
QVQLQESGPGLVAPSQTLSLTCTVSGFSLTSYGVSWIRQPPGKGLEWIGVIWEDGSTNYHSALISRVSISVDTSKSQFSLKLSSVDAADTAVYYCARPHYGSSYVGAMEYWGQGTTVTVSS

SEQ ID NO: 6 - protein, 107 aa, Artificial
  A04 CAR VL
DIQMTQSPSSLSASVGDRVTITCRASENIYSYVAWYQQKPGKAPKFLIYNAKSLAEGVPSRFSGSGSGTDFTLTISSLQPEDFANYYCQHHYVSPWTFGQGTKVEIK

SEQ ID NO: 7 - protein, 121 aa, Artificial
  A05 CAR VH
QVQLKESGPGLVAPSQTLSLTCTVSGFSLTSYGVSWIRQPPGKGLEWIGVIWEDGSTNYHSALISRVSISVDTSKSQFSLKLSSVDAADTAVYYCARPHYGSSYVGAMEYWGQGTTVTVSS

SEQ ID NO: 8 - protein, 107 aa, Artificial; identical to SEQ ID NO: 6
  A05 CAR VL

SEQ ID NO: 9 - protein, 121 aa, Artificial
  A06 CAR VH
QVQLKESGPGLVAPSQTLSLTCTVSGFSLTSYGVSWIRQPPGKGLEWIGVIWEDGSTNYHSALISRVSISVDTSKSQFSLKLSSVDAADTAVYYCAKPHYGSSYVGAMEYWGQGTTVTVSS

SEQ ID NO: 10 - protein, 107 aa, Artificial
  A06 CAR VL
DIQMTQSPSSLSASVGDRVTITCRASENIYSYVAWYQQKPGKAPKLLIYNAKSLAEGVPSRFSGSGSGTDFTLTISSLQPEDFANYYCQHHYVSPWTFGQGTKVEIK

SEQ ID NO: 11 - protein, 121 aa, Artificial; identical to SEQ ID NO: 9
  A07 CAR VH

SEQ ID NO: 12 - protein, 107 aa, Artificial; identical to SEQ ID NO: 6
  A07 CAR VL

SEQ ID NO: 13 - protein, 121 aa, Artificial
  A08 CAR VH
QVQLQESGPGLVKPSQTLSLTCTVSGFSLTSYGVSWIRQPPGKGLEWIGVIWEDGSTNYHSALISRVSISVDTSKSQFSLKLSSVDAADTAVYYCARPHYGSSYVGAMEYWGQGTTVTVSS

SEQ ID NO: 14 - protein, 107 aa, Artificial; identical to SEQ ID NO: 6
  A08 CAR VL

SEQ ID NO: 15 - protein, 120 aa, Artificial
  Anti-PD-1 scFv VH
QVQLVQSGAEVKKPGASVKVSCKASGYTFTNYYMYWVRQAPGQGLEWIGGINPSNGGTNFNEKFKNKATMTVDKSTSTAYMELSSLRSEDTAVYYCTRRDYNYDGGFDYWGQGTLVTVSS

SEQ ID NO: 16 - protein, 110 aa, Artificial
  Anti-PD-1 scFv VL
DIVLTQSPATLSLSPGERATLSCRASKSVSTSGFNYMHWYQQKPGQAPRLLIFLASNLASGVPARFSGSGSGTDFTLTISSLEPEDFAVYYCQHGRELPLTFGQGTKLEI

SEQ ID NO: 17 - DNA, 1506 bp, Artificial
  A02 CAR
atgctgctgctggtcacatctctgctgctgtgcgagctgccccatcctgcctttctgctgattcctcaggtgcagctgaaagagtctggccctggactggtggccccaagccagtctctgagcatcacctgtaccgtgtccggcttcagcctgacaagctatggcgtgtcctgggtccgacagcctcctagaaaaggcctggaatggctgggcgtgatctgggaggatggcagcaccaattaccacagcgccctgatcagcagactgtccatcaacaaggacaacagcaagagccaggtgttcctgaagctgaacagcctgcagaccgacgacaccgccacctactattgtgccaagcctcactacggcagcagctacgtgggcgctatggaatattggggccagggcacaagcgtgaccgtgtctagcgaaaagggaagaagtggcggcggaggatctggcggaggtggaagcggaggcggtggatctgatatccagatgacacagagccccgccagcctgactgcttctgtgggagagacagtgaccatcacatgccgggccagcgagaacatctactcctacgtggcctggtatcagcaaaagcagggcaagagccctcagttcctggtgtacaacgccaagtctctggccgagggcgtgccaagcagattttctggctctggcagcggcacccagttctccctgaagatcaattccctgcagccagaggacttcggcaactactactgccagcaccactatgtgtctccctggacctttggcggaggcaccaagctggaaatcaaacggtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacggtag

SEQ ID NO: 18 - protein, 501 aa, Artificial
  A02 CAR
MLLLVTSLLLCELPHPAFLLIPQVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSSEKGRSGGGGSGGGGSGGGGSDIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIKRYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 1506 bp, Artificial
  A03 CAR
atgctgctgctggtcacatctctgctgctgtgcgagctgccccatcctgcctttctgctgattcctcaggtccagctgcaagagtctggccctggactcgtcagacctagccagacactgagcctgacctgtaccgtgtccggcttcacctttaccagctacggcgtgtcctgggtccgacagcctcctggaagaggactggaatggatcggcgtgatctgggaggacggcagcaccaattatcacagcgccctgatcagcagagtgaccatgctggtggacacctccaagaaccagttcagcctgagactgagcagcgtgacagccgccgatacagccgtgtactactgtgccagacctcactacggcagcagctatgtgggcgccatggaatattggggccagggctctctggtcaccgtgtctagcgaaaagggaagaagtggcggcggaggatctggcggaggtggaagcggaggcggtggatctgatatccagatgacacagagccccagcagcctgtctgcctctgtgggagacagagtgacaatcacctgtcgggccagcgagaacatctacagctacgtggcctggtatcagcaaaagcccggcaaggcccctaagctgctgatctacaatgccaagtctctggccgagggcgtgcccagcagattttctggttctggcagcggcaccgactttacctttacaatcagctccctgcagcctgaggatatcgccacctactactgccagcaccactacgtgtccccatggacatttggccagggcaccaaggtggaaatcaagcggtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacggtag

SEQ ID NO: 20 - protein, 501 aa, Artificial
  A03 CAR
MLLLVTSLLLCELPHPAFLLIPQVQLQESGPGLVRPSQTLSLTCTVSGFTFTSYGVSWVRQPPGRGLEWIGVIWEDGSTNYHSALISRVTMLVDTSKNQFSLRLSSVTAADTAVYYCARPHYGSSYVGAMEYWGQGSLVTVSSEKGRSGGGGSGGGGSGGGGSDIQMTQSPSSLSASVGDRVTITCRASENIYSYVAWYQQKPGKAPKLLIYNAKSLAEGVPSRFSGSGSGTDFTFTISSLQPEDIATYYCQHHYVSPWTFGQGTKVEIKRYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 1503 bp, Artificial
  A04 CAR
atgctgctgctggtcacatctctgctgctgtgcgagctgccccatcctgcctttctgctgattcctcaggtccagctgcaagagtctggccctggactggttgccccaagccaaacactgagcctgacctgtaccgtgtccggctttagcctgacaagctacggcgtgtcctggatcagacagcctcctggcaaaggcctggaatggatcggagtgatctgggaggacggcagcaccaattaccacagcgccctgatcagccgggtgtccatctctgtggataccagcaagagccagttcagcctgaagctgtctagcgtggacgccgccgatacagccgtgtactattgtgccagacctcactacggcagcagctacgtgggcgctatggaatattggggccagggcaccaccgtgaccgtgtctagtgaaaagggaagatccggcggaggcggatctggtggcggaggtagtggcggcggaggatctgacattcagatgacacagagccccagcagcctgtctgcctctgtgggagacagagtgaccatcacctgtcgggccagcgagaacatctactcctacgtggcctggtatcagcaaaagcccggcaaggcccctaagttcctgatctacaacgccaagtctctggccgagggcgtgccaagcagattttctggctctggcagcggcaccgacttcaccctgacaatatctagcctccagcctgaggacttcgccaactactactgccagcaccactatgtgtctccctggacctttggccagggaacaaaggtggaaatcaagtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacggtag

SEQ ID NO: 22 - protein, 500 aa, Artificial
  A04 CAR
MLLLVTSLLLCELPHPAFLLIPQVQLQESGPGLVAPSQTLSLTCTVSGFSLTSYGVSWIRQPPGKGLEWIGVIWEDGSTNYHSALISRVSISVDTSKSQFSLKLSSVDAADTAVYYCARPHYGSSYVGAMEYWGQGTTVTVSSEKGRSGGGGSGGGGSGGGGSDIQMTQSPSSLSASVGDRVTITCRASENIYSYVAWYQQKPGKAPKFLIYNAKSLAEGVPSRFSGSGSGTDFTLTISSLQPEDFANYYCQHHYVSPWTFGQGTKVEIKYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1503 bp, Artificial
  A05 CAR
atgctgctgctggtcacatctctgctgctgtgcgagctgccccatcctgcctttctgctgattcctcaggtgcagctgaaagagtctggccctggactggtggccccaagccaaacactgagcctgacctgtaccgtgtccggctttagcctgacaagctacggcgtgtcctggatcagacagcctcctggcaaaggcctggaatggatcggagtgatctgggaggacggcagcaccaattaccacagcgccctgatcagccgggtgtccatctctgtggataccagcaagagccagttcagcctgaagctgtctagcgtggacgccgccgatacagccgtgtactattgtgccagacctcactacggcagcagctacgtgggcgctatggaatattggggccagggcaccaccgtgaccgtgtctagtgaaaagggaagatccggcggaggcggatctggtggcggaggtagtggcggcggaggatctgacattcagatgacacagagccccagcagcctgtctgcctctgtgggagacagagtgaccatcacctgtcgggccagcgagaacatctactcctacgtggcctggtatcagcaaaagcccggcaaggcccctaagttcctgatctacaacgccaagtctctggccgagggcgtgccaagcagattttctggctctggcagcggcaccgacttcaccctgacaatatctagcctccagcctgaggacttcgccaactactactgccagcaccactatgtgtctccctggacctttggccagggaacaaaggtggaaatcaagtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacggtag

SEQ ID NO: 24 - protein, 500 aa, Artificial
  A05 CAR
MLLLVTSLLLCELPHPAFLLIPQVQLKESGPGLVAPSQTLSLTCTVSGFSLTSYGVSWIRQPPGKGLEWIGVIWEDGSTNYHSALISRVSISVDTSKSQFSLKLSSVDAADTAVYYCARPHYGSSYVGAMEYWGQGTTVTVSSEKGRSGGGGSGGGGSGGGGSDIQMTQSPSSLSASVGDRVTITCRASENIYSYVAWYQQKPGKAPKFLIYNAKSLAEGVPSRFSGSGSGTDFTLTISSLQPEDFANYYCQHHYVSPWTFGQGTKVEIKYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 1503 bp, Artificial
  A06 CAR
atgctgctgctggtcacatctctgctgctgtgcgagctgccccatcctgcctttctgctgattcctcaggtgcagctgaaagagtctggccctggactggtggccccaagccaaacactgagcctgacctgtaccgtgtccggctttagcctgacaagctacggcgtgtcctggatcagacagcctcctggcaaaggcctggaatggatcggagtgatctgggaggacggcagcaccaattaccacagcgccctgatcagccgggtgtccatctctgtggataccagcaagagccagttcagcctgaagctgtctagcgtggacgccgccgatacagccgtgtactattgtgccaagcctcactacggcagcagctacgtgggcgctatggaatattggggccagggcaccaccgtgaccgtgtctagtgaaaagggaagatccggcggaggcggatctggtggcggaggtagtggcggcggaggatctgacattcagatgacacagagccccagcagcctgtctgcctctgtgggagacagagtgaccatcacctgtcgggccagcgagaacatctactcctacgtggcctggtatcagcaaaagcccggcaaggcccctaagctgctgatctacaatgccaagtctctggccgagggcgtgcccagcagattttctggttctggcagcggcaccgacttcaccctgacaatatctagcctccagcctgaggacttcgccaactactactgccagcaccactatgtgtctccctggacctttggccagggaacaaaggtggaaatcaagtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacggtag

SEQ ID NO: 26 - protein, 500 aa, Artificial
  A06 CAR
MLLLVTSLLLCELPHPAFLLIPQVQLKESGPGLVAPSQTLSLTCTVSGFSLTSYGVSWIRQPPGKGLEWIGVIWEDGSTNYHSALISRVSISVDTSKSQFSLKLSSVDAADTAVYYCAKPHYGSSYVGAMEYWGQGTTVTVSSEKGRSGGGGSGGGGSGGGGSDIQMTQSPSSLSASVGDRVTITCRASENIYSYVAWYQQKPGKAPKLLIYNAKSLAEGVPSRFSGSGSGTDFTLTISSLQPEDFANYYCQHHYVSPWTFGQGTKVEIKYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1503 bp, Artificial
  A07 CAR
atgctgctgctggtcacatctctgctgctgtgcgagctgccccatcctgcctttctgctgattcctcaggtgcagctgaaagagtctggccctggactggtggccccaagccaaacactgagcctgacctgtaccgtgtccggctttagcctgacaagctacggcgtgtcctggatcagacagcctcctggcaaaggcctggaatggatcggagtgatctgggaggacggcagcaccaattaccacagcgccctgatcagccgggtgtccatctctgtggataccagcaagagccagttcagcctgaagctgtctagcgtggacgccgccgatacagccgtgtactattgtgccaagcctcactacggcagcagctacgtgggcgctatggaatattggggccagggcaccaccgtgaccgtgtctagtgaaaagggaagatccggcggaggcggatctggtggcggaggtagtggcggcggaggatctgacattcagatgacacagagccccagcagcctgtctgcctctgtgggagacagagtgaccatcacctgtcgggccagcgagaacatctactcctacgtggcctggtatcagcaaaagcccggcaaggcccctaagttcctgatctacaacgccaagtctctggccgagggcgtgccaagcagattttctggctctggcagcggcaccgacttcaccctgaccatatctagcctgcagccagaggacttcgccaactactactgccagcaccactatgtgtctccctggacctttggccagggaacaaaggtggaaatcaagtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacggtag

SEQ ID NO: 28 - protein, 500 aa, Artificial
  A07 CAR
MLLLVTSLLLCELPHPAFLLIPQVQLKESGPGLVAPSQTLSLTCTVSGFSLTSYGVSWIRQPPGKGLEWIGVIWEDGSTNYHSALISRVSISVDTSKSQFSLKLSSVDAADTAVYYCAKPHYGSSYVGAMEYWGQGTTVTVSSEKGRSGGGGSGGGGSGGGGSDIQMTQSPSSLSASVGDRVTITCRASENIYSYVAWYQQKPGKAPKFLIYNAKSLAEGVPSRFSGSGSGTDFTLTISSLQPEDFANYYCQHHYVSPWTFGQGTKVEIKYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 1503 bp, Artificial
  A08 CAR
atgctgctgctggtcacatctctgctgctgtgcgagctgccccatcctgcctttctgctgattcctcaggtccagctgcaagagtctggccctggactggtcaagcctagccagacactgagcctgacctgtaccgtgtccggctttagcctgacaagctacggcgtgtcctggatcagacagcctcctggcaaaggcctggaatggatcggagtgatctgggaggacggcagcaccaattaccacagcgccctgatcagccgggtgtccatctctgtggataccagcaagagccagttcagcctgaagctgtctagcgtggacgccgccgatacagccgtgtactattgtgccagacctcactacggcagcagctacgtgggcgctatggaatattggggccagggcaccaccgtgaccgtgtctagtgaaaagggaagatccggcggaggcggatctggtggcggaggtagtggcggcggaggatctgacattcagatgacacagagccccagcagcctgtctgcctctgtgggagacagagtgaccatcacctgtcgggccagcgagaacatctactcctacgtggcctggtatcagcaaaagcccggcaaggcccctaagttcctgatctacaacgccaagtctctggccgagggcgtgccaagcagattttctggctctggcagcggcaccgacttcaccctgaccatatctagcctgcagcctgaggacttcgccaactactactgccagcaccactatgtgtctccctggacctttggccagggaacaaaggtggaaatcaagtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacggtag

SEQ ID NO: 30 - protein, 500 aa, Artificial
  A08 CAR
MLLLVTSLLLCELPHPAFLLIPQVQLQESGPGLVKPSQTLSLTCTVSGFSLTSYGVSWIRQPPGKGLEWIGVIWEDGSTNYHSALISRVSISVDTSKSQFSLKLSSVDAADTAVYYCARPHYGSSYVGAMEYWGQGTTVTVSSEKGRSGGGGSGGGGSGGGGSDIQMTQSPSSLSASVGDRVTITCRASENIYSYVAWYQQKPGKAPKFLIYNAKSLAEGVPSRFSGSGSGTDFTLTISSLQPEDFANYYCQHHYVSPWTFGQGTKVEIKYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 118 aa, Artificial [sequence omitted]

SEQ ID NO: 32 - protein, 107 aa, Artificial [sequence omitted]

SEQ ID NO: 33 - DNA, 2382 bp, Artificial
  A02P03
atgctgctgctggtcacatctctgctgctgtgcgagctgccccatcctgcctttctgctgattcctcaggtgcagctgaaagagtctggccctggactggtggccccaagccagtctctgagcatcacctgtaccgtgtccggcttcagcctgacaagctatggcgtgtcctgggtccgacagcctcctagaaaaggcctggaatggctgggcgtgatctgggaggatggcagcaccaattaccacagcgccctgatcagcagactgtccatcaacaaggacaacagcaagagccaggtgttcctgaagctgaacagcctgcagaccgacgacaccgccacctactattgtgccaagcctcactacggcagcagctacgtgggcgctatggaatattggggccagggcacaagcgtgaccgtgtctagcgaaaagggaagaagtggcggcggaggatctggcggaggtggaagcggaggcggtggatctgatatccagatgacacagagccccgccagcctgactgcttctgtgggagagacagtgaccatcacatgccgggccagcgagaacatctactcctacgtggcctggtatcagcaaaagcagggcaagagccctcagttcctggtgtacaacgccaagtctctggccgagggcgtgccaagcagattttctggctctggcagcggcacccagttctccctgaagatcaattccctgcagccagaggacttcggcaactactactgccagcaccactatgtgtctccctggacctttggcggaggcaccaagctggaaatcaaacggtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacgggtcgacagagccaagcggggctctggcgagggcagaggctctctgctgacctgcggagatgtggaagaaaatcccggccctatgtacagaatgcagctgttgtcttgtattgccctttctctcgccctcgtaacaaattcacaagtccagcttgtacaaagcggtgccgaagtgaaaaaaccgggcgcaagcgtcaaggtttcatgcaaggcgagtggatacacctttaccaactattacatgtactgggtgcggcaggcacccgggcagggactggaatggatcggtggtattaacccaagtaatggtggtacaaatttcaacgaaaagtttaagaacaaagcgacgatgacagtagacaaatctacaagtaccgcatacatggagctgagctcactgagatctgaggacaccgcagtctattactgcacacggcgagattacaattacgatggagggttcgactactggggccaagggactttggtgactgtatcctccggcggcggagggtcaggtggaggaggaagcggtggaggaggtagtgacatcgtccttacgcaaagcccggcgaccctcagtttgagccctggggaaagagcaacgctgtcttgtagagcatccaaatcagtctcaacttctggttttaattacatgcactggtatcaacagaagccaggacaagcacctagattgctgatctttctggcgtcaaatctggcctctggcgttccagcccggtttagcggttctggaagcggaacggactttacccttaccatatcatcactcgaaccagaggactttgcggtatattactgccaacatggacgggagctgcccttgacgtttggtcaaggaacgaagttggagatataa

SEQ ID NO: 34 - protein, 793 aa, Artificial
  A02P03
MLLLVTSLLLCELPHPAFLLIPQVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSSEKGRSGGGGSGGGGSGGGGSDIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIKRYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVDRAKRGSGEGRGSLLTCGDVEENPGPMYRMQLLSCIALSLALVTNSQVQLVQSGAEVKKPGASVKVSCKASGYTFTNYYMYWVRQAPGQGLEWIGGINPSNGGTNFNEKFKNKATMTVDKSTSTAYMELSSLRSEDTAVYYCTRRDYNYDGGFDYWGQGTLVTVSSGGGGSGGGGSGGGGSDIVLTQSPATLSLSPGERATLSCRASKSVSTSGFNYMHWYQQKPGQAPRLLIFLASNLASGVPARFSGSGSGTDFTLTISSLEPEDFAVYYCQHGRELPLTFGQGTKLEI

SEQ ID NO: 35 - DNA, 2367 bp, Artificial
  A02PL01
atgctgctgctggtcacatctctgctgctgtgcgagctgccccatcctgcctttctgctgattcctcaggtgcagctgaaagagtctggccctggactggtggccccaagccagtctctgagcatcacctgtaccgtgtccggcttcagcctgacaagctatggcgtgtcctgggtccgacagcctcctagaaaaggcctggaatggctgggcgtgatctgggaggatggcagcaccaattaccacagcgccctgatcagcagactgtccatcaacaaggacaacagcaagagccaggtgttcctgaagctgaacagcctgcagaccgacgacaccgccacctactattgtgccaagcctcactacggcagcagctacgtgggcgctatggaatattggggccagggcacaagcgtgaccgtgtctagcgaaaagggaagaagtggcggcggaggatctggcggaggtggaagcggaggcggtggatctgatatccagatgacacagagccccgccagcctgactgcttctgtgggagagacagtgaccatcacatgccgggccagcgagaacatctactcctacgtggcctggtatcagcaaaagcagggcaagagccctcagttcctggtgtacaacgccaagtctctggccgagggcgtgccaagcagattttctggctctggcagcggcacccagttctccctgaagatcaattccctgcagccagaggacttcggcaactactactgccagcaccactatgtgtctccctggacctttggcggaggcaccaagctggaaatcaaacggtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacgggtcgacagagccaagcggggctctggcgagggcagaggctctctgctgacctgcggagatgtggaagaaaatcccggccctatgtacagaatgcagctgttgtcttgtattgccctttctctcgccctcgtaacaaattcagatatccagatgacacaatctccgagctccttgtccgcatcagtgggggatagagttaccataacttgccgcgcgtctcaggatgtctccactgctgtcgcgtggtatcagcagaagccgggaaaggccccgaagttgctcatttactccgctagttttttgtattccggcgttcccagtcggttttctgggtcaggcagcggtaccgactttacacttacgattagttctctgcagcctgaggatttcgccacctattattgtcagcagtatttgtaccaccctgcaactttcggccagggaacaaaggtggaaataaagggcggtggtggaagtggaggagggggatccggaggcgggggttctgaagtccaattggttgagtccggcggaggccttgtccaaccagggggatcacttcggctgtcatgcgcggccagtggtttcacgtttagtgacagctggattcattgggtaagacaggcgcccgggaaaggtctcgagtgggtagcttggatttctccttacggaggttctacctattatgcggattctgttaaaggtcgattcacgatctctgcagatactagcaagaatacggcgtatcttcaaatgaatagccttcgggctgaggacaccgcggtgtactactgtgcccgaagacattggccgggcggattcgattattggggtcagggcactcttgtgacggtcagctcataa

SEQ ID NO: 36 - protein, 788 aa, Artificial
  A02PL01
MLLLVTSLLLCELPHPAFLLIPQVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSSEKGRSGGGGSGGGGSGGGGSDIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIKRYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVDRAKRGSGEGRGSLLTCGDVEENPGPMYRMQLLSCIALSLALVTNSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 37 - protein, 20 aa, Artificial
  Linker peptide
EKGRSGGGGSGGGGSGGGGS

SEQ ID NO: 38 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 39 - protein, 47 aa, Homo sapiens
RFSVVKRGRKKLLYIFKQPFMRPVQTTQEEDGCSCRFPEEEEGGCEL

SEQ ID NO: 40 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 21 aa, Artificial
  T2A
GSGEGRGSLLTCGDVEENPGP

SEQ ID NO: 42 - protein, 20 aa, Homo sapiens
MYRMQLLSCIALSLALVTNS

SEQ ID NO: 43 - protein, 245 aa, Artificial
  Anti-PD-1 scFv
QVQLVQSGAEVKKPGASVKVSCKASGYTFTNYYMYWVRQAPGQGLEWIGGINPSNGGTNFNEKFKNKATMTVDKSTSTAYMELSSLRSEDTAVYYCTRRDYNYDGGFDYWGQGTLVTVSSGGGGSGGGGSGGGGSDIVLTQSPATLSLSPGERATLSCRASKSVSTSGFNYMHWYQQKPGQAPRLLIFLASNLASGVPARFSGSGSGTDFTLTISSLEPEDFAVYYCQHGRELPLTFGQGTKLEI

SEQ ID NO: 44 - protein, 240 aa, Artificial
  Anti-PD-L1 scFv
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 45 - protein, 8 aa, Artificial
  CDR sequence
GFSLTSYG

SEQ ID NO: 46 - protein, 7 aa, Artificial
  CDR sequence
IWEDGST

SEQ ID NO: 47 - protein, 15 aa, Artificial
  CDR sequence
AKPHYGSSYVGAMEY

SEQ ID NO: 48 - protein, 6 aa, Artificial
  CDR sequence
ENIYSY

SEQ ID NO: 49 - protein, 3 aa, Artificial
  CDR sequence
NAK

SEQ ID NO: 50 - protein, 9 aa, Artificial
  CDR sequence
QHHYVSPWT

SEQ ID NO: 51 - protein, 8 aa, Artificial
  CDR sequence
GFTFTSYG

SEQ ID NO: 52 - protein, 7 aa, Artificial
  CDR sequence
IWEDGST

SEQ ID NO: 53 - protein, 15 aa, Artificial
  CDR sequence
ARPHYGSSYVGAMEY

SEQ ID NO: 54 - protein, 6 aa, Artificial
  CDR sequence
ENIYSY

SEQ ID NO: 55 - protein, 3 aa, Artificial
  CDR sequence
NAK

SEQ ID NO: 56 - protein, 9 aa, Artificial
  CDR sequence
QHHYVSPWT

SEQ ID NO: 57 - protein, 8 aa, Artificial
  CDR sequence
GFSLTSYG

SEQ ID NO: 58 - protein, 7 aa, Artificial
  CDR sequence
IWEDGST

SEQ ID NO: 59 - protein, 15 aa, Artificial
  CDR sequence
ARPHYGSSYVGAMEY

SEQ ID NO: 60 - protein, 6 aa, Artificial
  CDR sequence
ENIYSY

SEQ ID NO: 61 - protein, 3 aa, Artificial
  CDR sequence
NAK

SEQ ID NO: 62 - protein, 9 aa, Artificial
  CDR sequence
QHHYVSPWT

SEQ ID NO: 63 - protein, 8 aa, Artificial
  CDR sequence
GFSLTSYG

SEQ ID NO: 64 - protein, 7 aa, Artificial
  CDR sequence
IWEDGST

SEQ ID NO: 65 - protein, 15 aa, Artificial
  CDR sequence
ARPHYGSSYVGAMEY

SEQ ID NO: 66 - protein, 6 aa, Artificial
  CDR sequence
ENIYSY

SEQ ID NO: 67 - protein, 3 aa, Artificial
  CDR sequence
NAK

SEQ ID NO: 68 - protein, 9 aa, Artificial
  CDR sequence
QHHYVSPWT

SEQ ID NO: 69 - protein, 8 aa, Artificial
  CDR sequence
GFSLTSYG

SEQ ID NO: 70 - protein, 7 aa, Artificial
  CDR sequence
IWEDGST

SEQ ID NO: 71 - protein, 15 aa, Artificial
  CDR sequence
AKPHYGSSYVGAMEY

SEQ ID NO: 72 - protein, 6 aa, Artificial
  CDR sequence
ENIYSY

SEQ ID NO: 73 - protein, 3 aa, Artificial
  CDR sequence
NAK

SEQ ID NO: 74 - protein, 9 aa, Artificial
  CDR sequence
QHHYVSPWT

SEQ ID NO: 75 - protein, 8 aa, Artificial
  CDR sequence
GFSLTSYG

SEQ ID NO: 76 - protein, 7 aa, Artificial
  CDR sequence
IWEDGST

SEQ ID NO: 77 - protein, 15 aa, Artificial
  CDR sequence
AKPHYGSSYVGAMEY

SEQ ID NO: 78 - protein, 6 aa, Artificial
  CDR sequence
ENIYSY

SEQ ID NO: 79 - protein, 3 aa, Artificial
  CDR sequence
NAK

SEQ ID NO: 80 - protein, 9 aa, Artificial
  CDR sequence
QHHYVSPWT

SEQ ID NO: 81 - protein, 8 aa, Artificial
  CDR sequence
GFSLTSYG

SEQ ID NO: 82 - protein, 7 aa, Artificial
  CDR sequence
IWEDGST

SEQ ID NO: 83 - protein, 15 aa, Artificial
  CDR sequence
ARPHYGSSYVGAMEY

SEQ ID NO: 84 - protein, 6 aa, Artificial
  CDR sequence
ENIYSY

SEQ ID NO: 85 - protein, 3 aa, Artificial
  CDR sequence
NAK

SEQ ID NO: 86 - protein, 9 aa, Artificial
  CDR sequence
QHHYVSPWT

SEQ ID NO: 87 - protein, 15 aa, Artificial
  Linker peptide
GGGGSGGGGSGGGGS

SEQ ID NO: 88 - protein, 5 aa, Artificial
  Linker peptide
GGGGS

SEQ ID NO: 89 - protein, 41 aa, Homo sapiens
RSKRSRLLHSDYMNMTPRRPGPTRKHYQPYAPPRDFAAYRS

SEQ ID NO: 90 - protein, 88 aa, Artificial
  CD28 and 4-1BB intracellular signaling domain
RSKRSRLLHSDYMNMTPRRPGPTRKHYQPYAPPRDFAAYRSRFSVVKRGRKKLLYIFKQPFMRPVQTTQEEDGCSCRFPEEEEGGCEL

SEQ ID NO: 91 - protein, 542 aa, Artificial
  A02 third generation CAR
MLLLVTSLLLCELPHPAFLLIPQVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSSEKGRSGGGGSGGGGSGGGGSDIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIKRYVTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 542 aa, Artificial
  A03 third generation CAR
MLLLVTSLLLCELPHPAFLLIPQVQLQESGPGLVRPSQTLSLTCTVSGFTFTSYGVSWVRQPPGRGLEWIGVIWEDGSTNYHSALISRVTMLVDTSKNQFSLRLSSVTAADTAVYYCARPHYGSSYVGAMEYWGQGSLVTVSSEKGRSGGGGSGGGGSGGGGSDIQMTQSPSSLSASVGDRVTITCRASENIYSYVAWYQQKPGKAPKLLIYNAKSLAEGVPSRFSGSGSGTDFTFTISSLQPEDIATYYCQHHYVSPWTFGQGTKVEIKRYVTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 541 aa, Artificial
  A06 third generation CAR
MLLLVTSLLLCELPHPAFLLIPQVQLKESGPGLVAPSQTLSLTCTVSGFSLTSYGVSWIRQPPGKGLEWIGVIWEDGSTNYHSALISRVSISVDTSKSQFSLKLSSVDAADTAVYYCAKPHYGSSYVGAMEYWGQGTTVTVSSEKGRSGGGGSGGGGSGGGGSDIQMTQSPSSLSASVGDRVTITCRASENIYSYVAWYQQKPGKAPKLLIYNAKSLAEGVPSRFSGSGSGTDFTLTISSLQPEDFANYYCQHHYVSPWTFGQGTKVEIKYVTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 47 aa, Homo sapiens
KPTTTPAPRPPTPAPTIASQPLSLRPEACRPAAGGAVHTRGLDFACD

SEQ ID NO: 95 - protein, 45 aa, Homo sapiens
TTTPAPRPPTPAPTIASQPLSLRPEACRPAAGGAVHTRGLDFACD

SEQ ID NO: 96 - protein, 22 aa, Homo sapiens
MALIVLGGVAGLLLFIGLGIFF

SEQ ID NO: 97 - protein, 39 aa, Homo sapiens
IEVMYPPPYLDNEKSNGTIIHVKGKHLCPSPLFPGPSKP

SEQ ID NO: 98 - protein, 108 aa, Artificial
  A02 CAR VL-2
DIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIRR

SEQ ID NO: 99 - protein, 501 aa, Artificial
  A02 CAR-2
MLLLVTSLLLCELPHPAFLLIPQVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSSEKGRSGGGGSGGGGSGGGGSDIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIRRYVTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 1503 bp, Artificial
  A02 CAR-2
atgctgctgctggtcacatctctgctgctgtgcgagctgccccatcctgcctttctgctgattcctcaggtgcagctgaaagagtctggccctggactggtggccccaagccagtctctgagcatcacctgtaccgtgtccggcttcagcctgacaagctatggcgtgtcctgggtccgacagcctcctagaaaaggcctggaatggctgggcgtgatctgggaggatggcagcaccaattaccacagcgccctgatcagcagactgtccatcaacaaggacaacagcaagagccaggtgttcctgaagctgaacagcctgcagaccgacgacaccgccacctactattgtgccaagcctcactacggcagcagctacgtgggcgctatggaatattggggccagggcacaagcgtgaccgtgtctagcgaaaagggaagaagtggcggcggaggatctggcggaggtggaagcggaggcggtggatctgatatccagatgacacagagccccgccagcctgactgcttctgtgggagagacagtgaccatcacatgccgggccagcgagaacatctactcctacgtggcctggtatcagcaaaagcagggcaagagccctcagttcctggtgtacaacgccaagtctctggccgagggcgtgccaagcagattttctggctctggcagcggcacccagttctccctgaagatcaattccctgcagccagaggacttcggcaactactactgccagcaccactatgtgtctccctggacctttggcggaggcaccaagctggaaatcagacggtacgtaacgacgacaccagcaccccgccctcccacaccagcgcctacgatcgccagccaaccattgtcactgcggcctgaggcttgcagaccagctgccggtggagccgtccacacacgcggacttgactttgcttgcgacatctacatctgggctcctctcgcaggcacctgcggtgtactgcttctctcactggtcattacactctattgccgatttagtgtggtcaaacgaggccgaaaaaaactcctttatatcttcaaacagcctttcatgagacctgtgcaaacaacccaggaagaagacggttgctcttgccgctttcccgaagaagaagaaggaggttgcgaactgagagttaaattctctaggtctgccgacgcccctgcctaccagcagggccagaaccagctgtataacgaactgaacctcggacgacgagaagaatatgatgtactcgataaaagacgcggaagggaccccgaaatgggcgggaagccccggaggaagaacccacaggagggtctgtataatgaactccagaaagacaaaatggccgaagcatattcagaaattggcatgaaaggggaacgaaggcggggtaaagggcacgatggcctttatcagggacttagcacagccacaaaagacacttatgacgctctgcacatgcaagccctccccccacgg

SEQ ID NO: 101 - protein, 501 aa, Artificial
  A02-8H CAR
MLLLVTSLLLCELPHPAFLLIPQVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSSEKGRSGGGGSGGGGSGGGGSDIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIRRYVKPTTTPAPRPPTPAPTIASQPLSLRPEACRPAAGGAVHTRGLDFACDMALIVLGGVAGLLLFIGLGIFFRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 1503 bp, Artificial
  A02-8H CAR
atgctgctgctggtcacatctctgctgctgtgcgagctgccccatcctgcctttctgctgattcctcaggtgcagctgaaagagtctggccctggactggtggccccaagccagtctctgagcatcacctgtaccgtgtccggcttcagcctgacaagctatggcgtgtcctgggtccgacagcctcctagaaaaggcctggaatggctgggcgtgatctgggaggatggcagcaccaattaccacagcgccctgatcagcagactgtccatcaacaaggacaacagcaagagccaggtgttcctgaagctgaacagcctgcagaccgacgacaccgccacctactattgtgccaagcctcactacggcagcagctacgtgggcgctatggaatattggggccagggcacaagcgtgaccgtgtctagcgaaaagggaagaagtggcggcggaggatctggcggaggtggaagcggaggcggtggatctgatatccagatgacacagagccccgccagcctgactgcttctgtgggagagacagtgaccatcacatgccgggccagcgagaacatctactcctacgtggcctggtatcagcaaaagcagggcaagagccctcagttcctggtgtacaacgccaagtctctggccgagggcgtgccaagcagattttctggctctggcagcggcacccagttctccctgaagatcaattccctgcagccagaggacttcggcaactactactgccagcaccactatgtgtctccctggacctttggcggaggcaccaagctggaaatcagacggtacgtaaagcctaccaccacaccagctcctagacctccaactcctgctcctacaatcgccagccagcctctgtctctcagacccgaagcctgtagacctgctgctggcggagctgtgcataccagaggactggatttcgcctgcgacatggccctgattgtgcttggcggagttgctggactgctgctgtttatcggcctgggcatcttcttcagattcagcgtggtcaagcggggcagaaagaagctgctgtacatcttcaagcagcccttcatgcggcccgtgcagaccacacaagaggaagatggctgctcctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 103 - protein, 493 aa, Artificial
  A02-28H CAR
MLLLVTSLLLCELPHPAFLLIPQVQLKESGPGLVAPSQSLSITCTVSGFSLTSYGVSWVRQPPRKGLEWLGVIWEDGSTNYHSALISRLSINKDNSKSQVFLKLNSLQTDDTATYYCAKPHYGSSYVGAMEYWGQGTSVTVSSEKGRSGGGGSGGGGSGGGGSDIQMTQSPASLTASVGETVTITCRASENIYSYVAWYQQKQGKSPQFLVYNAKSLAEGVPSRFSGSGSGTQFSLKINSLQPEDFGNYYCQHHYVSPWTFGGGTKLEIRRYVIEVMYPPPYLDNEKSNGTIIHVKGKHLCPSPLFPGPSKPMALIVLGGVAGLLLFIGLGIFFRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - DNA, 1479 bp, Artificial
  A02-28H CAR
atgctgctgctggtcacatctctgctgctgtgcgagctgccccatcctgcctttctgctgattcctcaggtgcagctgaaagagtctggccctggactggtggccccaagccagtctctgagcatcacctgtaccgtgtccggcttcagcctgacaagctatggcgtgtcctgggtccgacagcctcctagaaaaggcctggaatggctgggcgtgatctgggaggatggcagcaccaattaccacagcgccctgatcagcagactgtccatcaacaaggacaacagcaagagccaggtgttcctgaagctgaacagcctgcagaccgacgacaccgccacctactattgtgccaagcctcactacggcagcagctacgtgggcgctatggaatattggggccagggcacaagcgtgaccgtgtctagcgaaaagggaagaagtggcggcggaggatctggcggaggtggaagcggaggcggtggatctgatatccagatgacacagagccccgccagcctgactgcttctgtgggagagacagtgaccatcacatgccgggccagcgagaacatctactcctacgtggcctggtatcagcaaaagcagggcaagagccctcagttcctggtgtacaacgccaagtctctggccgagggcgtgccaagcagattttctggctctggcagcggcacccagttctccctgaagatcaattccctgcagccagaggacttcggcaactactactgccagcaccactatgtgtctccctggacctttggcggaggcaccaagctggaaatcagacggtacgtaatcgaagtgatgtaccctcctccttacctggacaacgagaagtccaacggcaccatcatccacgtgaagggcaagcacctgtgtccttctccactgttccccggacctagcaagcctatggctctgattgtgcttggcggagtggctggcctgctgctgtttatcggcctgggcatcttcttcagattcagcgtggtcaagcggggcagaaagaagctgctgtacatcttcaagcagcccttcatgcggcccgtgcagaccacacaagaggaagatggctgctcctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

(19) United States
(12) Patent Application Publication
(10) Pub. No.: US 2022/0125845 A1
(43) Pub. Date: Apr. 28, 2022

(54) ANTI-ALPP CAR-T CELL THERAPY

(71) Applicants: GUANGDONG TCRCURE BIOPHARMA TECHNOLOGY CO., LTD., Guangzhou (CN); TCRCURE BIOPHARMA CORP., Chapel Hill, NC (US)

(72) Inventors: Rui CHEN, Durham, NC (US); Peter ALEXANDER, Durham, NC (US); Lixia ZHAO, Cary, NC (US); Brooke WOLFF, Haw River, NC (US); Rhiannon ROARK, Durham, NC (US)

(21) Appl. No.: 17/561,089

(22) Filed: Dec. 23, 2021

Related U.S. Application Data
(63) Continuation-in-part of application No. PCT/US2020/039084, filed on Jun. 23, 2020.
(60) Provisional application No. 62/865,244, filed on Jun. 23, 2019.

(51) Int. Cl. A61K 35/17; A61P 35/00; C07K 14/725; C07K 14/705; C07K 16/40

(52) U.S. Cl. CPC A61K 35/17; A61P 35/00; C07K 16/40; C07K 14/70517; C07K 14/70578; C07K 14/7051

(57) ABSTRACT

The disclosure relates to anti-ALPP CAR-T cell therapies for the treatment of cancer patients having ALPP-positive cancer, including e.g., ovarian, endometrial, cervical, testicular cancers, etc.

### CLAIM OF PRIORITY

This application is a continuation-in-part of International Application PCT/US2020/039084, with an international filing date of Jun. 23, 2020, which claims the benefit of U.S. Provisional Application No. 62/865,244, filed on Jun. 23, 2019. The entire contents of the foregoing applications are incorporated herein by reference.

### TECHNICAL FIELD

The disclosure relates to anti-ALPP CAR-T cell therapy for the treatment of cancer patients.

### BACKGROUND

Ovarian cancer accounts for approximately 3% of all cancers in women and is the fifth leading cause of cancer-related death among women in the United States. Due to the lack of early symptoms and of effective ovarian cancer screening tests, it has the highest mortality of all cancers of the female reproductive system. Numerous associations have been reported between the expression of ALPP (Alkaline Phosphatase, Placental) and ovarian cancer. In normal tissues, ALPP is only detectable in term placenta and endometrium. By contrast, ALPP is strongly expressed in ovarian cancer, especially in ovarian adenocarcinoma, serous cystadenocarcinoma, undifferentiated carcinoma and dysgerminoma. In addition, ALPP is also detected in some other malignancies, particularly in testicular seminoma and endometrial cancer.

The recent clinical and commercial success of immune cell therapy has created great interest in this area. Despite this advancement in cancer treatments, the efficacy of various treatments against certain cancers remains relatively poor. Accordingly, there exists an unmet need for effective anti-cancer therapies.

### SUMMARY

The disclosure relates to anti-ALPP CAR-T cell therapy for the treatment of cancer patients with ALPP-positive cancer, including e.g., ovarian, endometrial, cervical, testicular cancers, etc. Genetically engineered T cells can recognize and attack target cells. These T cells can be isolated from the host and genetically modified using e.g., suitable virus mediated or non-viral means of transfection. Thereafter, the modified T cells can be infused back into the patients as adoptive cell therapy.

In one aspect, the disclosure is related to a method of treating cancer, comprising administering an effective amount of genetically engineered anti-tumor human T cells to a patient to treat cancer of the patient. In some embodiments, the anti-tumor human T cells have been obtained by incorporating a recombinant DNA sequence encoding a CAR into T cells extracted from the patient. In some embodiments, the encoded CAR comprises an ALPP antigen binding domain that binds to ALPP expressed in cancer cells.

In some embodiments, the cancer is lung cancer. In some embodiments, the cancer is gastric cancer. In some embodiments, the cancer is pancreatic cancer. In some embodiments, the cancer is head & neck cancer. In some embodiments, the cancer is colorectal cancer. In some embodiments, the cancer is urothelial cancer. In some embodiments, the cancer is renal cancer. In some embodiments, the cancer is cancer of reproductive organs. In some embodiments, the cancer of reproductive organs is ovarian cancer. In some embodiments, the cancer of reproductive organs is endometrial cancer. In some embodiments, the cancer of reproductive organs is cervical cancer. In some embodiments, the cancer of reproductive organs is testicular cancer.

In one aspect, the disclosure is related to a method of treating cancer, comprising administering an effective amount of genetically engineered anti-tumor human T cells to a patient to treat cancer of the patient. In some embodiments, the anti-tumor human T cells have been obtained by incorporating a recombinant DNA sequence encoding a CAR into T cells extracted from the patient. In some embodiments, the encoded CAR comprises an ALPP antigen binding domain. In some embodiments, the CAR-T cell antigen binding domain consists of an antibody or antibody fragment. In some embodiments, the said antibody has a variable heavy chain region selected from SEQ ID NO: 1 or SEQ ID NO: 3; and a variable light chain region selected from SEQ ID NO: 2 or SEQ ID NO: 4.

In one aspect, the disclosure is related to the method of treating cancer as described herein. In some embodiments, the antibody is murine antibody against ALPP having a variable heavy chain region SEQ ID NO: 1 and variable light chain region SEQ ID NO: 2 or 98.

In one aspect, the disclosure is related to the method of treating cancer as described herein. In some embodiments, the antibody is humanized antibody against ALPP having a variable heavy chain region SEQ ID NO: 3 and variable light chain region SEQ ID NO: 4.

In one aspect, the disclosure is related to a chimeric antigen receptor comprising: (a) an extracellular antigen-binding domain that specifically recognizes alkaline phosphatase, placental (ALPP); (b) a transmembrane domain; and (c) an intracellular signaling region.

In some embodiments, the antigen-binding domain comprises a heavy chain variable domain (VH) and a light chain variable domain (VL).

In some embodiments, the VH comprises heavy chain complementarity determining regions (CDRs) 1, 2, and 3 and the VL comprises VL CDRs 1, 2, and 3. In some embodiments, the VH CDRs 1, 2, and 3 amino acid sequences and the VL CDRs, 1, 2, and 3 amino acid sequences are one of the following:
(1) the VH CDRs 1, 2, and 3 amino acid sequences are set forth in SEQ ID NOs: 45, 46, and 47, respectively, and the VL CDRs 1, 2, and 3 amino acid sequences are set forth in SEQ ID NOs: 48, 49, and 50, respectively;
(2) the VH CDRs 1, 2, and 3 amino acid sequences are set forth in SEQ ID Nos: 51, 52, and 53, respectively, and the VL CDRs 1, 2, and 3 amino acid sequences are set forth in SEQ ID NOs: 54, 55, and 56, respectively;
(3) the VH CDRs 1, 2, and 3 amino acid sequences are set forth in SEQ ID NOs: 57, 58, and 59, respectively, and the VL CDRs 1, 2, and 3 amino acid sequences are set forth in SEQ ID NOs: 60, 61, and 62, respectively;
(4) the VH CDRs 1, 2, and 3 amino acid sequences are set forth in SEQ ID NOs: 63, 64, and 65, respectively, and the VL CDRs 1, 2, and 3 amino acid sequences are set forth in SEQ ID NOs: 66, 67, and 68, respectively; and
(5) the VH CDRs 1, 2, and 3 amino acid sequences are set forth in SEQ ID NOs: 81, 82, and 83, respectively, and the VL CDRs 1, 2, and 3 amino acid sequences are set forth in SEQ ID NOs: 84, 85, and 86, respectively.

In some embodiments, the VH consist of or comprises an amino acid sequence having at least 90%, 91%, 92%, 93%, 94%, 95%, 96%, 97%, 98%, 99% or 100% sequence identity to SEQ ID NOs: 1, 3, 5, 7, 9, 11, or 13; and the VL consists of or comprises an amino acid sequence having at least 90%, 91%, 92%, 93%, 94%, 95%, 96%, 97%, 98%, 99% or 100% sequence identity to SEQ ID NOs: 2, 4, 6, 8, 10, 12, 14, or 98.

In some embodiments, the VH comprises an amino acid sequence having at least 90% identity to SEQ ID NO: 1 and the VL comprises an amino acid sequence having at least 90% identity to SEQ ID NO: 2.

In some embodiments, the VH comprises an amino acid sequence having at least 90% identity to SEQ ID NO: 1 and the VL comprises an amino acid sequence having at least 90% identity to SEQ ID NO: 98.

In some embodiments, the VH comprises an amino acid sequence having at least 90% identity to SEQ ID NO: 3 and the VL comprises an amino acid sequence having at least 90% identity to SEQ ID NO: 4.

In some embodiments, the VH comprises an amino acid sequence having at least 90% identity to SEQ ID NO: 5 and the VL comprises an amino acid sequence having at least 90% identity to SEQ ID NO: 6.

In some embodiments, the VH comprises an amino acid sequence having at least 90% identity to SEQ ID NO: 7 and the VL comprises an amino acid sequence having at least 90% identity to SEQ ID NO: 8.

In some embodiments, the VH comprises an amino acid sequence having at least 90% identity to SEQ ID NO: 9 and the VL comprises an amino acid sequence having at least 90% identity to SEQ ID NO: 10.

In some embodiments, the VH comprises an amino acid sequence having at least 90% identity to SEQ ID NO: 11 and the VL comprises an amino acid sequence having at least 90% identity to SEQ ID NO: 12.

In some embodiments, the VH comprises an amino acid sequence having at least 90% identity to SEQ ID NO: 13 and the VL comprises an amino acid sequence having at least 90% identity to SEQ ID NO: 14.

In some embodiments, the antigen-binding domain comprises an scFv.

In some embodiments, the VH region and the VL region are joined by a flexible linker.

In some embodiments, the flexible linker comprises the amino acid sequence of EKGRSGGGGSGGGGSGGGGS (SEQ ID NO: 37). In some embodiments, the flexible linker comprises the amino acid sequence of GGGGSGGGGSGGGGS (SEQ ID NO: 87).

In some embodiments, the chimeric antigen receptor further comprises a hinge region. In some embodiments, the hinge region comprises a membrane-proximal region from IgG, CD8, or CD28. In some embodiments, the hinge region comprises a CD8 membrane-proximal region. In some embodiments, the CD8 membrane-proximal region comprises an amino acid sequence set forth in SEQ ID NO: 94 or 95, or an amino acid sequence that is at least 90% identical to SEQ ID NO: 94 or 95. In some embodiments, the hinge region comprises a CD28 (e.g., human CD28) membrane-proximal region. In some embodiments, the CD28 membrane-proximal region comprises an amino acid sequence set forth in SEQ ID NO: 97, or an amino acid sequence that is at least 90% identical to SEQ ID NO: 97.

In some embodiments, the transmembrane domain comprises a transmembrane region of CD4, CD8, or CD28. In some embodiments, the transmembrane domain comprises a CD8 transmembrane region. In some embodiments, the hinge region and/or the transmembrane region are from human CD8. In some embodiments, the chimeric antigen receptor comprises an amino acid sequence set forth in SEQ ID NO: 38, or an amino acid sequence that is at least 90% identical to SEQ ID NO: 38. In some embodiments, the transmembrane domain comprises a CD4 (e.g., human CD4) transmembrane region. In some embodiments, the CD4 transmembrane region comprises an amino acid sequence set forth in SEQ ID NO: 96, or an amino acid sequence that is at least 90% identical to SEQ ID NO: 96.

In some embodiments, the intracellular signaling region comprises an activating cytoplasmic signaling domain. In some embodiments, the activating cytoplasmic signaling domain is capable of inducing a primary activation signal in a T cell, is a T cell receptor (TCR) component, and/or comprises an immunoreceptor tyrosine-based activation motif (ITAM). In some embodiments, the intracellular signaling region is or comprises a functional signaling domain of CD3 zeta. In some embodiments, the CD3 zeta is human CD3 zeta. In some embodiments, the intracellular signaling region is or comprises the amino acid sequence set forth in SEQ ID NO: 40 or an amino acid sequence that is at least 90% sequence identical to SEQ ID NO: 40.

In some embodiments, the intracellular signaling region further comprises a costimulatory signaling region. In some embodiments, the costimulatory signaling region is between the transmembrane domain and the intracellular signaling region.

In some embodiments, the costimulatory signaling region comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In some embodiments, the costimulatory signaling region is or comprises a functional signaling domain from OX40, CD28, 4-1BB, ICOS, or a signaling portion thereof. In some embodiments, the costimulatory signaling region comprises an intracellular signaling domain of 4-1BB. In some embodiments, the 4-1BB is human 4-1BB. In some embodiments, the costimulatory signaling region is or comprises an amino acid sequence set forth in SEQ ID NO: 39 or an amino acid sequence that is at least 90% identical to SEQ ID NO: 39.

In some embodiments, the costimulatory signaling region comprises intracellular signaling domains of CD28 and 4-1BB. In some embodiments, the CD28 is human CD28 and the 4-1BB is human 4-1BB. In some embodiments, the costimulatory signaling region is or comprises an amino acid sequence set forth in SEQ ID NO: 90 or an amino acid sequence that is at least 90% identical to SEQ ID NO: 90.

In one aspect, the disclosure is related to a chimeric antigen receptor comprising an amino acid sequence having at least 90%, 91%, 92%, 93%, 94%, 95%, 96%, 97%, 98%, 99% or 100% identity to SEQ ID NO: 18, 20, 22, 24, 26, 28, 30, 91, 92, 93, 99, 101, or 103.

In some embodiments, the amino acid sequence is at least 90%, 91%, 92%, 93%, 94%, 95%, 96%, 97%, 98%, 99% or 100% identical to SEQ ID NO: 18, 99, 101, or 103. In some embodiments, the amino acid sequence is at least 90%, 91%, 92%, 93%, 94%, 95%, 96%, 97%, 98%, 99% or 100% identical to SEQ ID NO: 20. In some embodiments, the amino acid sequence is at least 90%, 91%, 92%, 93%, 94%, 95%, 96%, 97%, 98%, 99% or 100% identical to SEQ ID NO: 22. In some embodiments, the amino acid sequence is at least 90%, 91%, 92%, 93%, 94%, 95%, 96%, 97%, 98%, 99% or 100% identical to SEQ ID NO: 24. In some embodiments, the amino acid sequence is at least 90%, 91%, 92%, 93%, 94%, 95%, 96%, 97%, 98%, 99% or 100% identical to SEQ ID NO: 26. In some embodiments, the amino acid sequence is at least 90%, 91%, 92%, 93%, 94%, 95%, 96%, 97%, 98%, 99% or 100% identical to SEQ ID NO: 28. In some embodiments, the amino acid sequence is at least 90%, 91%, 92%, 93%, 94%, 95%, 96%, 97%, 98%, 99% or 100% identical to SEQ ID NO: 30. In some embodiments, the amino acid sequence is at least 90%, 91%, 92%, 93%, 94%, 95%, 96%, 97%, 98%, 99% or 100% identical to SEQ ID NO: 91. In some embodiments, the amino acid sequence is at least 90%, 91%, 92%, 93%, 94%, 95%, 96%, 97%, 98%, 99% or 100% identical to SEQ ID NO: 92. In some embodiments, the amino acid sequence is at least 90%, 91%, 92%, 93%, 94%, 95%, 96%, 97%, 98%, 99% or 100% identical to SEQ ID NO: 93.

In one aspect, the disclosure is related to a polynucleotide encoding the chimeric antigen receptor as described herein.

In one aspect, the disclosure is related to a vector comprising the polynucleotide as described herein.

In some embodiments, the vector further comprises a nucleic acid encoding an anti-PD-1 antibody or antigen binding fragment thereof. In some embodiments, the vector further comprises a nucleic acid encoding an anti-PD-L1 antibody or antigen binding fragment thereof.

In some embodiments, the vector is a viral vector. In some embodiments, the viral vector is a retroviral vector or a lentiviral vector.

In one aspect, the disclosure is related to an engineered cell, comprising the chimeric antigen receptor as described herein.

In one aspect, the disclosure is related to an engineered cell, comprising the polynucleotide or the vector as described herein.

In some embodiments, the engineered cell is a primary cell obtained from a subject (e.g., a human subject). In some embodiments, the engineered cell is a cell line. In some embodiments, the engineered cell is an immune cell. In some embodiments, the immune cell is an NK cell or a T cell. In some embodiments, the engineered cell is a T cell. In some embodiments, the T cell is CD8+. In some embodiments, the T cell is CD4+. In some embodiments, the T cell is isolated from a human subject.

In some embodiments, the engineered cell expresses the chimeric antigen receptor.

In some embodiments, the engineered cell expresses a cytokine and/or a co-stimulatory ligand. In some embodiments, the cytokine and/or the co-stimulatory ligand is membrane tethered. In some embodiments, the cytokine and/or the co-stimulatory ligand is secreted. In some embodiments, the cytokine is IL-2, IL-5, or IL-12. In some embodiments, the co-stimulatory ligand is CD40L (CD154) or 41-BBL (CD137L). In some embodiments, the engineered cell expresses an antibody or antigen-binding fragment thereof (e.g., an scFv).

In some embodiments, the antibody or antigen-binding fragment thereof is an immune checkpoint inhibitor. In some embodiments, the antibody or antigen-binding fragment thereof specifically binds to PD-1, PD-L1, or CTLA-4.

In one aspect, the disclosure is related to a method for producing the engineered cell, comprising introducing a vector as described herein into a cell in vitro or ex vivo.

In some embodiments, the vector is a viral vector and the introducing is carried out by transduction.

In one aspect, the disclosure is related to a method of generating a population of cells, comprising introducing a nucleic acid into a cell, where the nucleic acid comprises the polynucleotide as described herein, or a nucleic acid encoding the chimeric antigen receptor as described herein.

In one aspect, the disclosure is related to a method of treating an ALPP-associated disease or disorder in a subject, comprising administering the engineered cell as described herein to the subject.

In some embodiments, the ALPP-associated disease or disorder is a cancer. In some embodiments, the cancer is testicular cancer, endometrial cancer, ovarian cancer, cervical cancer, urothelial cancer, pancreatic cancer, liver cancer, or stomach cancer.

In some embodiments, the method further comprises administering a checkpoint inhibitor to the subject. In some embodiments, the checkpoint inhibitor is an anti-PD-1 antibody or antigen binding fragment thereof, an anti-PD-L1 antibody or antigen binding fragment thereof, or an anti-CTLA-4 antibody or antigen-binding fragment thereof

In one aspect, the disclosure is related to an anti-ALPP antibody or antigen-binding fragment thereof comprising: a heavy chain variable region (VH) comprising complementarity determining regions (CDRs) 1, 2, and 3 and a light chain variable region (VL) comprising CDRs 1, 2, and 3. In some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence In some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence. In some embodiments, the selected VH CDRs 1, 2, and 3 amino acid sequences and the selected VL CDRs, 1, 2, and 3 amino acid sequences are one of the following:
(1) the selected VH CDRs 1, 2, and 3 amino acid sequences are set forth in SEQ ID NOs: 51, 52, and 53, respectively, and the selected VL CDRs 1, 2, and 3 amino acid sequences are set forth in SEQ ID NOs: 54, 55, and 56, respectively;
(2) the selected VH CDRs 1, 2, and 3 amino acid sequences are set forth in SEQ ID NOs: 57, 58, and 59, respectively, and the selected VL CDRs 1, 2, and 3 amino acid sequences are set forth in SEQ ID NOs: 60, 61, and 62, respectively;
(3) the selected VH CDRs 1, 2, and 3 amino acid sequences are set forth in SEQ ID NOs: 63, 64, and 65, respectively, and the selected VL CDRs 1, 2, and 3 amino acid sequences are set forth in SEQ ID NOs: 66, 67, and 68, respectively; and
(4) the selected VH CDRs 1, 2, and 3 amino acid sequences are set forth in SEQ ID NOs: 81, 82, and 83, respectively, and the selected VL CDRs 1, 2, and 3 amino acid sequences are set forth in SEQ ID NOs: 84, 85, and 86, respectively.

In one aspect, the disclosure is related to an antibody or antigen-binding fragment thereof that binds to ALPP comprising a heavy chain variable region (VH) comprising an amino acid sequence that is at least 90%, 91%, 92%, 93%, 94%, 95%, 96%, 97%, 98%, 99%, or 100% identical to a selected VH sequence, and a light chain variable region (VL) comprising an amino acid sequence that is at least 90%, 91%, 92%, 93%, 94%, 95%, 96%, 97%, 98%, 99%, or 100% identical to a selected VL sequence. In some embodiments, the selected VH sequence is selected from SEQ ID NOs: 1, 3, 5, 7, 9, 11, and 13, and the selected VL sequence is selected from SEQ ID NOs: 2, 4, 6, 8, 10, 12, 14, or 98.

In some embodiments, the antibody or antigen-binding fragment specifically binds to human ALPP.

In some embodiments, the antibody or antigen-binding fragment is a humanized antibody or antigen-binding fragment thereof.

In some embodiments, the antibody or antigen-binding fragment is a single-chain variable fragment (scFv).

In one aspect, the disclosure is related to an antibody or antigen-binding fragment thereof comprising the VH CDRs 1, 2, and 3, and the VL CDRs 1, 2, and 3 of the antibody or antigen-binding fragment thereof as described herein.

In one aspect, the disclosure is related to a chimeric antigen receptor comprising the VH CDRs 1, 2, and 3, and the VL CDRs 1, 2, and 3 of the antibody or antigen-binding fragment thereof as described herein.

As used herein, the term “genetically engineered cell” or “genetically modified cell” refers to a cell with a modification of a nucleic acid sequence in the cell, including, but not limited to, a cell having a insertion, deletion, substitution, or modification of one or more nucleotides in its genome, and/or a cell with an exogenous nucleic acid sequence (e.g., a vector), wherein the exogenous nucleic acid sequence is not necessarily integrated into the genome.

As used herein, the term “peripheral blood cells” refers to cells normally found in the peripheral blood including, but is not limited to, eosinophils, neutrophils, T cells, monocytes, K cells, granulocytes, and B cells.

As used herein, the term “cancer” or “cancer cell” refers to the cells dividing in an uncontrolled manner, e.g., forming the solid tumors or the excessive tumor cells in blood. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. The cancer cells can form the solid tumors or the excessive tumor cells in blood (e.g., hematologic cancer). Alternatively or additionally it can include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Examples of cancers that can be treated by the methods described herein include e.g., bone cancer, pancreatic cancer, skin cancer (e.g., melanoma), cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, and/or T cell lymphoma.

As used herein, the term “vector” refers to a vehicle by which a polynucleotide sequence (e.g. a foreign gene) can be introduced into a host cell, in order to obtain the desired gene expression of the introduced nucleotide sequence. Cloning vectors can include e.g., plasmids, phages, viruses, etc. Most popular type of vector is a “plasmid”, which refers to a closed circular double stranded DNA loop into which additional DNA segments comprising gene of interest can be ligated. Another type of vector is a viral vector, in which a nucleic acid construct to be transported is ligated into the viral genome. Viral vectors are capable of autonomous replication in a host cell into which they are introduced or may integrate themselves into the genome of a host cell and thereby are replicated along with the host genome. Moreover, certain vectors are capable of directing the expression of genes to which they are operatively linked. Such vectors are referred to herein as “recombinant expression vectors” or simply “expression vectors”. In some embodiments, the vectors are viral vectors (e.g., replication defective retroviruses, adenoviruses and adeno-associated viruses). In some embodiments, the vector is a retroviral vector. In some embodiments, the vector can be created by removal of the retroviral gag, pol, and env genes and replaced with the gene of interest.

As used herein, a “subject” is a mammal, such as a human or a non-human animal. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a dog, a cat, a horse, a rodent, a rat, or a mouse.

As used herein, the term “T cells” refer to the cells that are a type of lymphocyte which develop in the thymus and play an important role in the immune response. T cells can be distinguished from other lymphocytes by the presence of a T cell receptor on the cell surface.

As used herein, the term “about” refers to a measurable value such as an amount, a time duration, and the like, and encompasses variations of ±20%, ±10%, ±5%, ±1%, ±0.5% or ±0.1% from the specified value.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)2, and Fv fragments.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, when referring to an antibody or antibody related molecule (e.g., CAR), the phrases “specifically binding” and “specifically binds” mean that the antibody interacts with its target molecule (e.g., ALPP) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to an ALPP molecule may be referred to as an ALPP-specific antibody or an anti-ALPP antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

Exemplary embodiments are illustrated in referenced figures. It is intended that the embodiments and figures disclosed herein are to be considered illustrative rather than restrictive.

FIG. 1A shows a sequence map for the A02 CAR plasmid.

FIG. 1B shows a sequence map for the A03 CAR plasmid.

FIG. 1C shows a sequence map for the A06 CAR plasmid.

FIG. 1D shows a sequence map for the A02P03 CAR plasmid. T2A encodes a 2A self-cleaving peptide.

FIG. 1E shows a sequence map for the A02PL01 CAR plasmid. T2A encodes a 2A self-cleaving peptide.

FIG. 2 shows A02 and A03 CAR expression in Jurkat T cells. Jurkat cells were untransduced (UT), or transduced to express A02 or A03 CAR. CAR expression levels were measured by protein L staining 4 days post-transduction.

FIG. 3 shows A02 and A03 CAR-T cell activation. Untransduced (UT) Jurkat cells and Jurkat cells only (Blank) served as controls.

FIG. 4 shows A02 and A03 CAR expression in human T cells. Human PBMCs were transduced to express A02 or A03 CAR. CAR expression was measured by protein L staining 4 days post-transduction.

FIG. 5 shows CD4⁺ A02 and A03 CAR-T cell activation. Intracellular IFN-γ expression was measured by flow cytometry in human CD4⁺ T cells. Untransduced (UT) T cells and T cells only (Blank) served as controls.

FIG. 6 shows CD8⁺ A02 and A03 CAR-T cell activation. Intracellular IFN-γ expression was measured by flow cytometry in human CD8⁺ T cells. Untransduced (UT) T cells and T cells only (Blank) served as controls.

FIG. 7 shows competitive killing activity of A02 and A03 CAR-T cells. Each 96-well plate contained 4 replicates. Live SiHa and 293T cells were analyzed by flow cytometry, and competitive killing efficiency was calculated based on numbers of live SiHa/293T cell ratio.

FIG. 8 shows in vivo toxicity of A02 and A03 CAR-T cells. In vivo toxicity was assessed by measuring body weight changes.

FIG. 9 shows in vivo antitumor efficacy of A02 and A03 CAR-T cells. ***, p=0.0009 compared with untransduced; *, p=0.0281 compared with untransduced.

FIG. 10A shows a set of binding curves of anti-ALPP antibodies binding to ALPP as determined by ELISA.

FIG. 10B shows a set of binding curves of anti-ALPP antibodies binding to ALPP as determined by cell-based binding assays.

FIG. 11A is a graph showing CAR expression levels of untransduced (UT), A02, A03, A05, A06, or A07 CAR-T cells, as determined by protein L assays.

FIG. 11B is a set of flow cytometry results showing IFNy expression in untransduced (UT), A02, A03, A05, A06, or A07 CAR-T cells. The CAR-T cells were co-cultured with SiHa or 293T cells. CD8⁺ cells were analyzed by flow cytometry.

FIG. 11C is a set of flow cytometry results showing IFNy expression in untransduced (UT), A02, A03, A05, A06, or A07 CAR-T cells. The CAR-T cells were co-cultured with SiHa or 293T cells. CD4⁺ cells were analyzed by flow cytometry.

FIG. 11D shows competitive killing curves at various effector-to-target cell ratios.

FIG. 12A is a graph showing CAR expression levels of untransduced (UT), A02, A02P03, or A02PL01 CAR-T cells, as determined by protein L assays.

FIG. 12B is a set of flow cytometry results showing IFNy expression in untransduced (UT), A02, A02P03, or A02PL01 CAR-T cells. The CAR-T cells were co-cultured with SiHa or 293T cells. CD8⁺ cells were analyzed by flow cytometry.

FIG. 12C is a set of flow cytometry results showing IFNy expression in untransduced (UT), A02, A02P03, or A02PL01 CAR-T cells. The CAR-T cells were co-cultured with SiHa or 293T cells. CD4⁺ cells were analyzed by flow cytometry.

FIG. 12D shows competitive killing curves at various effector-to-target cell ratios.

FIG. 13A shows survival curves of NSG mice (The Jackson Laboratory) implanted with SiHa cells and injected with untransduced, A02, A02P03, AO2PL01, A03, orA06 CAR-T cells.

FIG. 13B shows percentage of mouse body weight changes of NSG mice implanted with SiHa cells and injected with untransduced, A02, A02P03, A02PL01, A03, orA06 CAR-T cells.

FIG. 14 shows schematic diagrams of the A02, A02-8H, and A02-28H constructs.

FIG. 15 shows the expression of different ALPP CARs in human T cells.

FIG. 16 shows the basal activity of different ALPP CARs in human T cells.

FIG. 17 shows the in vitro proliferation of ALPP CAR-T cells.

FIG. 18A is a set of flow cytometry results showing IFN-γ expression in untransduced (UT), A02, A02-8H, or A02-28H CAR-T cells. The CAR-T cells were co-cultured without tumor cells (Blank), with non-target tumor cells (293T), or with target-tumor cells (SiHa). CD3⁺CD8⁺ cells were analyzed by flow cytometry.

FIG. 18B is a set of flow cytometry results showing IFN-γ expression in untransduced (UT), A02, A02-8H, or A02-28H CAR-T cells. The CAR-T cells were co-cultured without tumor cells (Blank), with non-target tumor cells (293T), or with target-tumor cells (SiHa). CD3+CD4+ cells were analyzed by flow cytometry.

FIG. 19 shows competitive killing curves at various effector-to-target cell ratios.

FIG. 20 shows CDR1, 2, 3 sequences of the heavy chain variable region (VH) and light chain variable region (VL).

FIG. 21 provides sequences that are described in the disclosure.

### DETAILED DESCRIPTION

The human immune system is capable of recognizing and eliminating cells that have become infected or damaged as well as those that have become cancerous. Immune cell therapy takes advantage of the human immune system and is revolutionizing cancer therapy. It involves the transfer of immune cells into a patient. The cells are most commonly derived from the immune system and can originate from the patient or from another individual. In autologous cancer immunotherapy, immune cells are extracted from the patient, genetically modified and cultured in vitro, and returned to the same patient. Comparatively, allogeneic therapies involve cells isolated and expanded from a donor subject. Many different kinds of immune cells are used in immune cell therapies. These cell therapies include e.g., tumor-infiltrating lymphocyte (TIL) therapy, engineered T cell receptor (TCR) therapy, chimeric antigen receptor (CAR) T cell therapy, and natural killer (NK) cell therapy.

Chimeric antigen receptor T cells (also known as CAR-T cells) are T cells that have been genetically engineered to produce an artificial T cell receptor for use in immunotherapy. In CAR-T cell therapy, the cells are transfected by a vector encoding a chimeric antigen receptor. The chimeric antigen receptor can bind to cancer antigens and does not require that the cancer antigens be presented by MHC. Some other immune cells can also be used in these cell therapies. For example, natural killer cells can also be transfected with a vector encoding a chimeric antigen receptor.

Placental alkaline phosphatase (PLAP or ALPP) is a plasma membrane-localized enzyme with normal human tissue expression restricted to the placenta, cervix, and uterus. However, ALPP is also expressed in ovarian, cervical, and testicular cancers, such that ALPP has been considered as a molecular marker for these tumor types. Theoretically, this restricted expression of ALPP in normal tissues and its upregulation in the above-mentioned cancers can be successfully exploited to improve cancer diagnosis and prognostic prediction, as well as to develop new treatments that lack major side effects.

The present disclosure relates to an anti-ALPP CAR-T cell therapy for treating cancer patients with ALPP-positive tumors. Further, the disclosure discloses a method for producing anti-ALPP CAR-T cells which can be used as cell-based therapies for patients with ALPP-positive cancers.

In one aspect, provided herein is an anti-ALPP CAR-T therapy as a novel treatment developed for patients with ALPP-positive tumors. This anti-ALPP CAR is engineered into patient-specific T cells, which can be delivered as a single therapeutic agent. Furthermore, these patient specific anti-ALPP CAR-T cells exhibit improved efficacy and specificity against a variety of ALPP-associated cancers. As mentioned above, ALPP expression is highly restricted in normal human tissues, suggesting that CAR-T cell therapy targeting this protein can be well tolerated. Indeed, studies conducted here using an animal model of cervical cancer demonstrate that anti-ALPP CAR-T cell therapy is in fact safe and efficacious.

In some embodiments, the present disclosure provides a method for personalized anti-tumor immunotherapy, wherein the anti-ALPP CAR engineered T cells are produced from a patient's blood. These engineered T cells can be re-infused into the patient as a cellular therapy product. Such a product can then be applied to any patient harboring an ALPP-positive tumor, including patients with ovarian, endometrial, cervical, or testicular cancers, among others.

In some embodiments, the present disclosure provides a method of producing engineered T cells, wherein these T cells are transfected with a retroviral vector containing an anti-ALPP CAR transgene and then expanded in vitro. These expanded cells are infused back into the patient, where the engineered T cells identify and destroy ALPP-positive tumor cells.

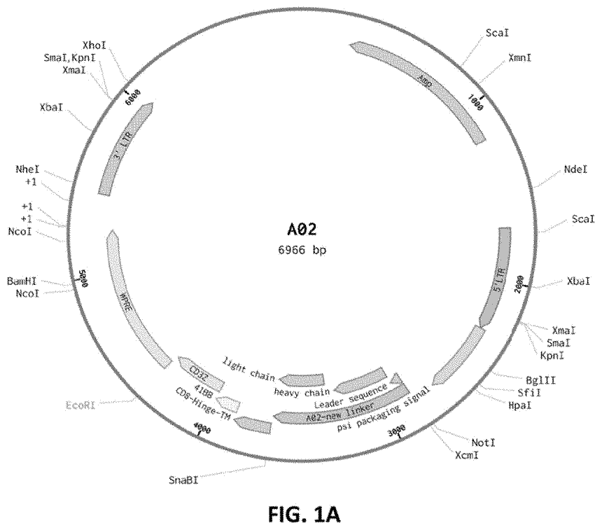

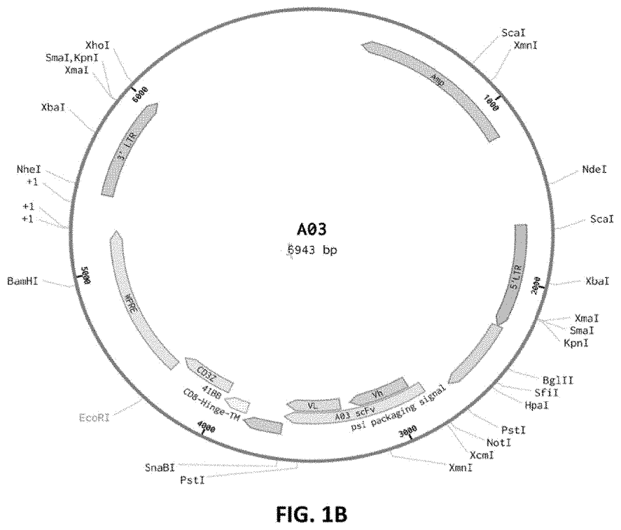

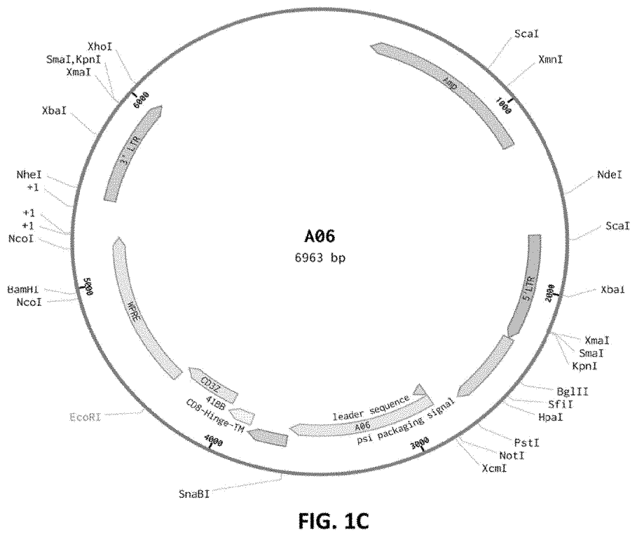

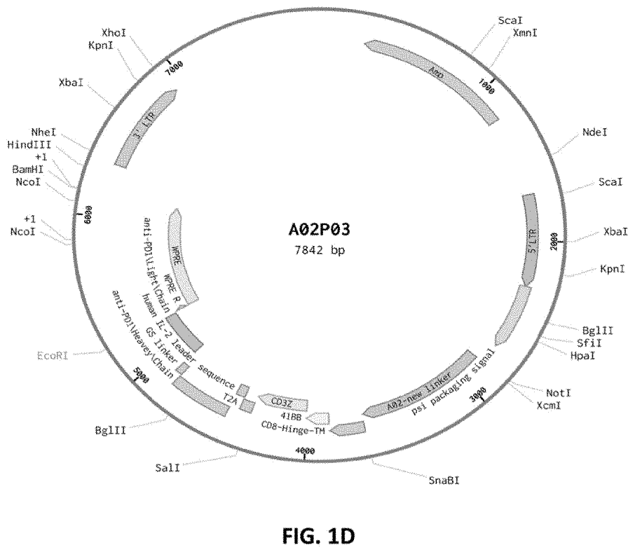

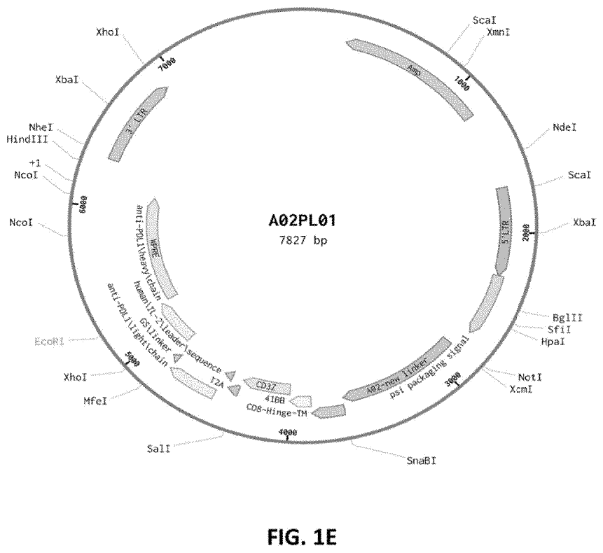

In some embodiments, MP71 retroviral constructs are generated using standard molecular biology techniques. Further, A02 (FIG. 1A) and A03 (FIG. 1B) discloses retroviral plasmid maps according to the present disclosure, wherein A02 (FIG. 1A) is based on the murine H17E2 monoclonal antibody recognizing ALPP, while A03 (FIG. 1B) is a humanized version of H17E2.

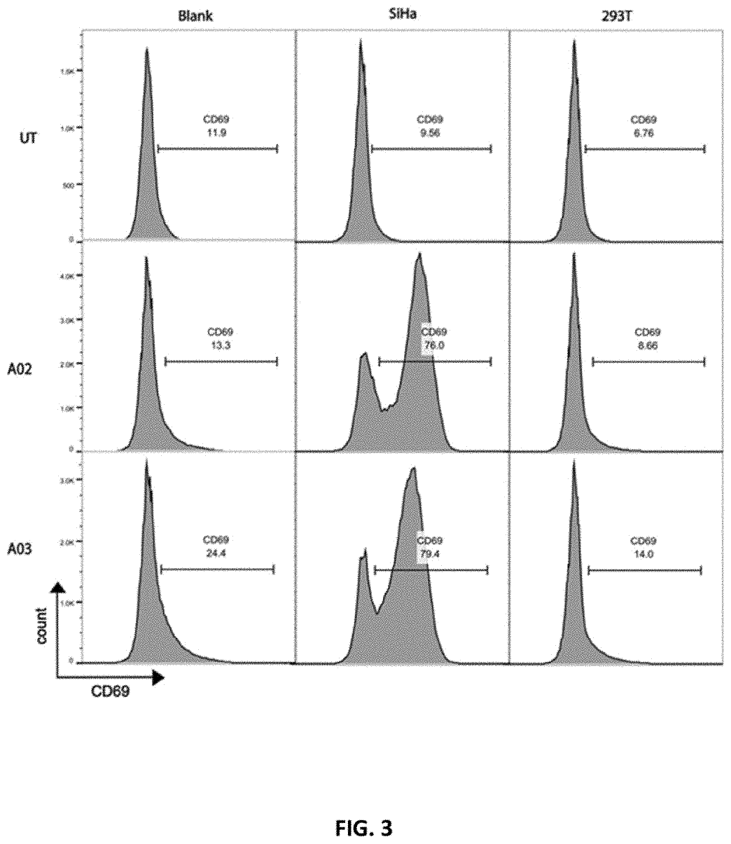

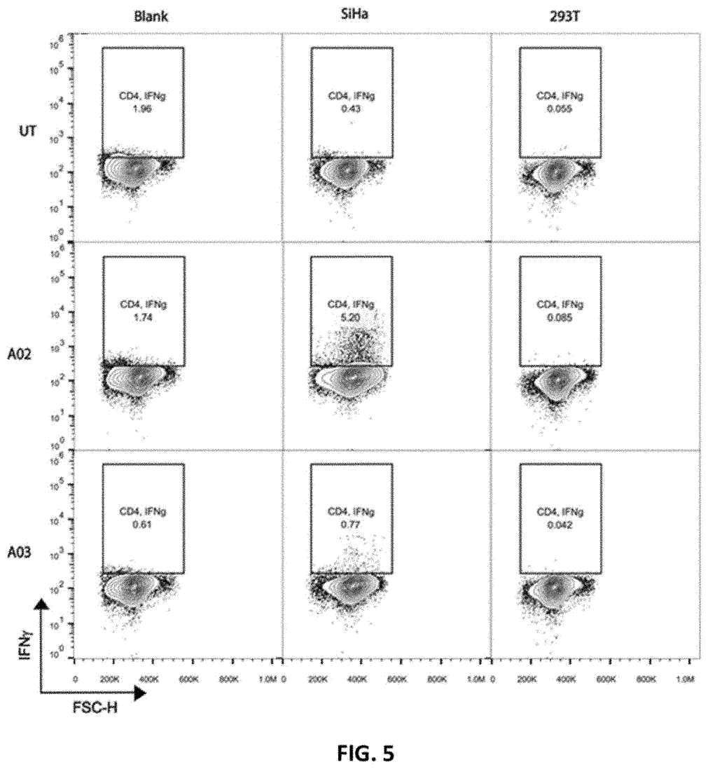

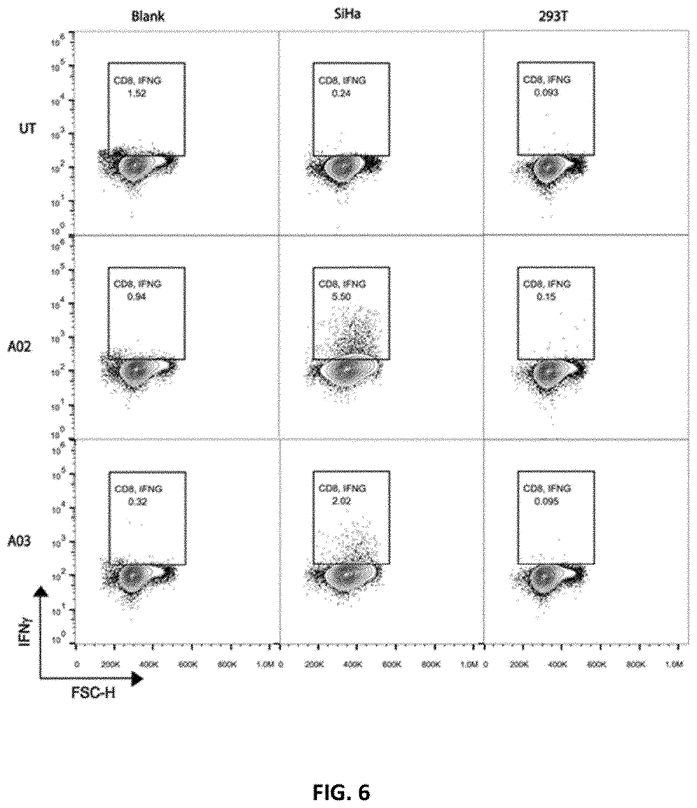

Furthermore, the present disclosure provides an in vitro method of detecting the effect of CAR-T cells on different types of cancer. CAR-T cell activation in response to the ALPP-antigen can be assessed using ALPP-positive SiHa cervical cancer cells, wherein both Jurkat and PBMC CAR-T cells are specifically activated upon co-culture with SiHa cells. CAR-T cell activation can be measured by expression of the established T cell activation markers CD69 (FIG. 3) and IFN-γ (FIGS. 5 and 6).

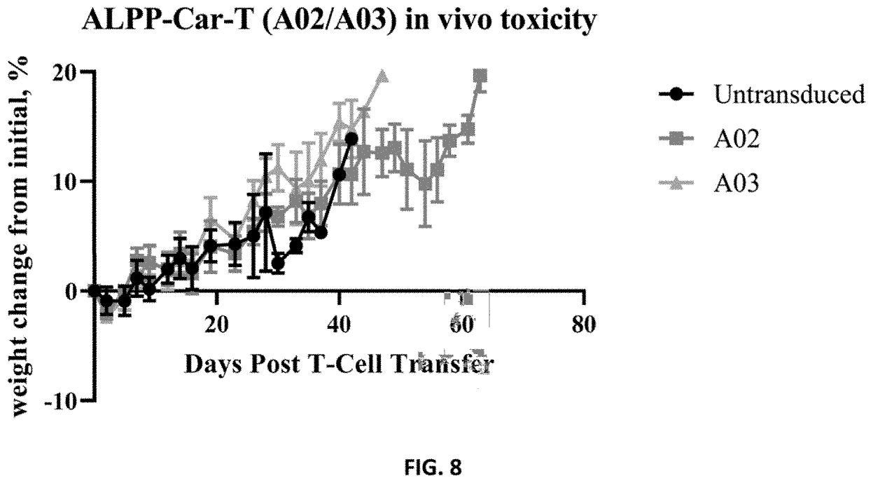

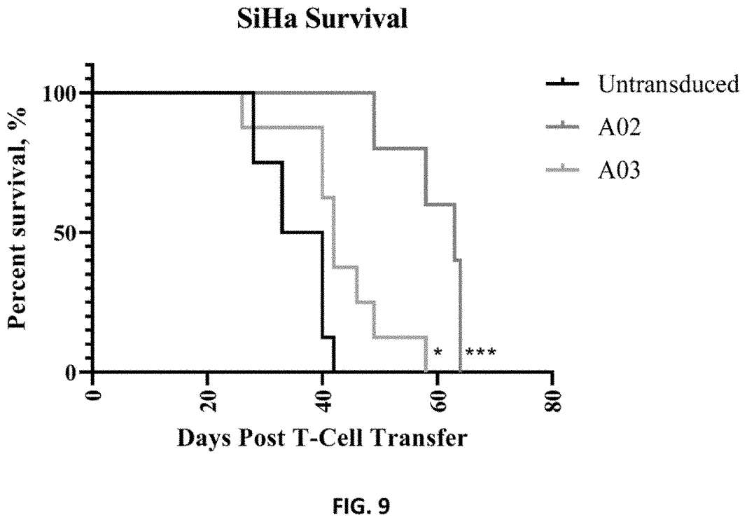

Furthermore, the present disclosure provides an in-vivo method of detecting the effect of CAR-T cells on different types of cancer. For this, SiHa cells were inoculated intraperitoneally into immunodeficient NSG mice to allow the formation of ALPP-positive tumors. Subsequently, A02, A03, or untransduced CAR-T cells were administered as anti-tumor therapy. Whereas tumors in mice treated with untransduced T cells progressed rapidly to cause animal death, treatment with either A02 or A03 CAR-T cells significantly improved animal survival (FIG. 9) with no observable toxicities caused by this treatment (FIG. 8). These findings suggest that anti-ALPP CAR-T cell therapy is a safe and effective treatment for ALPP-positive tumors of reproductive organs.

According to embodiments of the disclosure, anti-ALPP CAR-T cell therapy can be used for different types of cancer having elevated expression of ALPP, including, but not limited to, cancers of the cervix, ovaries, endometrium, uterus, testes, brain, thyroid, lung, pancreas, head & neck, stomach, colorectum, kidney, and urothelial cells.

According to some embodiments, the CAR comprises a single chain antibody fragment comprising an original murine anti-ALPP binding domain. The antibody comprises a variable heavy chain region having a sequence represented by SEQ ID NO: 1 and a variable light chain region having a sequence represented by SEQ ID NO: 2 or 98.

In some embodiments, the CAR comprises a humanized anti-ALPP binding domain. The antigen binding domain comprises a variable heavy chain region having a sequence represented by SEQ ID NO: 3 and a variable light chain region having a sequence represented by SEQ ID NO: 4.

In some embodiments, the retroviral plasmid A02 (FIG. 1A) transfected cells express a CAR having a murine anti-ALPP binding domain. The antibody (H17E2) fragment comprises a variable heavy chain region SEQ ID NO: 1 and a variable light chain region SEQ ID NO: 2 or 98.

In one aspect, the disclosure also provides a humanized antibody or antibody fragment having specificity for human ALPP. In some embodiments, the humanized antibody or antibody fragments are produced by transferring the Complementarity Determining Regions (CDRs) from a murine antibody (e.g., H17E2) into a human antibody variable region framework. This developed molecule can be used in the treatment or diagnosis of cancer.

In some embodiments, the retroviral plasmid A03 (FIG. 1B) transfected cells express a CAR having a humanized anti-ALPP binding domain. The humanized antibody fragment comprises a variable heavy chain region SEQ ID NO: 3 and a variable light chain region SEQ ID NO: 4.

Clinically, the anti-ALPP CAR can be engineered into patient-specific T cells and delivered as a single therapeutic agent. Based on the in vitro and in vivo results shown herein, this strategy can have improved efficacy and specificity against a variety of ALPP-associated cancers.

ALPP and Cancer

Alkaline phosphatase, placental (ALPP), also known as placental alkaline phosphatase (PLAP) (NCBI GENE ID: 250), is a plasma membrane-localized enzyme with normal human tissue expression restricted to the placenta, cervix, and uterus. ALPP is a homodimer, membrane-associated glycoprotein enzyme. It belongs to a multigene family composed of four alkaline phosphatase isoenzymes. The enzyme functions as a homodimer and has a catalytic site containing one magnesium and two zinc ions, which are required for its enzymatic function. It plays an important role in the regulation of specific inflammatory disease processes. There are at least four distinct but related alkaline phosphatases: intestinal, placental, placental-like, and liver/bone/kidney. Placental Alkaline Phosphatase (ALPP) reacts with a membrane-bound isoenzyme (Regan and Nagao type) of ALPP occurring in the placenta during the third trimester of gestation. Placental Alkaline Phosphatase is useful in the identification of testicular germ cell tumors. Unlike germ cell tumors, ALPP -positive somatic cell tumors uniformly express epithelial membrane antigen (EMA).

Elevated ALPP expression is commonly found in ovarian, cervical, and testicular cancers. ALPP expression has also been observed in testicular seminoma, primary intracranial germinoma, epithelial ovarian carcinoma, ovarian adenocarcinoma, serous cystadenocarcinoma, undifferentiated carcinoma, dysgerminoma, uterus cancer, endometrial cancer, urothelial cancer, stomach cancer, lung cancer, pancreatic cancer, osteosarcoma, and gastric cancer. Because of its restricted expression pattern, ALPP can be considered as both a molecular marker and a therapeutic target for ALPP-positive cancers.

The disclosure relates to anti-ALPP CAR-T cell therapy for the treatment of cancer patients with ALPP-positive cancer. The present disclosure also provides antibodies or antigen binding fragments that target ALPP.

### Chimeric Antigen Receptors and Binding Molecules

Chimeric antigen receptors (CARs) combine many facets of normal T cell activation into a single protein. They link an extracellular antigen recognition domain to an intracellular signaling domain, which activates the T cell when an antigen is bound. CARs are typically composed of four regions: an antigen binding domain, an extracellular hinge region, a transmembrane domain, and an intracellular T cell signaling domain.

The antigen binding domain is exposed to the outside of the cell, in the ectodomain portion of the receptor. It interacts with potential target molecules and is responsible for targeting the CAR-T cell to any cell expressing a matching molecule. The antigen binding domain is typically derived from the variable regions of a monoclonal antibody linked together as a single-chain variable fragment (scFv). An scFv is a chimeric protein made up of the light (VL) and heavy (VH) chains of immunoglobulins, connected with a short linker peptide. The linker between the two chains consists of hydrophilic residues with stretches of glycine and serine in it for flexibility as well as stretches of glutamate and lysine for added solubility. In some embodiments, the antigen binding domain specifically binds to a tumor associated antigen, e.g., BCMA, CD19, CD22, CD30, CD33, CD56, CD123 (also known as IL-3R), CEA, EBV-related antigens (e.g., LMP2), EGFR, GD2, GPC3, HER2, HPV-related antigens (e.g., E6), MAGE antigens, Mesothelin, MUC-1, NY-ESO-1, PSCA, PSMA, ROR1, WT1, or Claudin 18.2. In some embodiments, the antigen binding domain specifically binds to ALPP.

The hinge, also called a spacer, is a small structural domain that sits between the antigen binding domain and the cell's outer membrane. An ideal hinge enhances the flexibility of the scFv receptor head, reducing the spatial constraints between the CAR and its target antigen. This promotes antigen binding and synapse formation between the CAR-T cells and target cells. Hinge sequences are often based on membrane-proximal regions from immune molecules including e.g., IgG, CD8, and CD28.

The transmembrane domain is a structural component, consisting of a hydrophobic alpha helix that spans the cell membrane. It anchors the CAR to the plasma membrane, bridging the extracellular hinge and antigen binding domains with the intracellular signaling region. This domain is essential for the stability of the receptor as a whole. Generally, the transmembrane domain from the most membrane-proximal component of the endodomain is used, but different transmembrane domains result in different receptor stability. The CD28 transmembrane domain is known to result in a highly expressed, stable receptor.

The intracellular T cell signaling domain lies in the receptor's endodomain, inside the cell. After an antigen is bound to the external antigen binding domain, CAR receptors cluster together and transmit an activation signal. Then the internal cytoplasmic end of the receptor perpetuates signaling inside the T cell. Normal T cell activation relies on the phosphorylation of immunoreceptor tyrosine-based activation motifs (ITAMs) present in the cytoplasmic domain of CD3-zeta. To mimic this process, CD3-zeta's cytoplasmic domain is commonly used as the main CAR endodomain component. T cells also require co-stimulatory molecules in addition to CD3 signaling in order to persist after activation. For this reason, the endodomains of CAR receptors typically also include one or more chimeric domains from co-stimulatory proteins. Signaling domains from a wide variety of co-stimulatory molecules have been successfully tested, including CD28, CD27, CD134 (0X40), and CD137 (4-1BB).

Various CAR molecules and vectors expressing these CAR molecules can be used in the methods described herein. In some embodiments, the CAR molecules specifically binds to a tumor-associated antigen, e.g., ALPP. In some embodiments, the CAR comprises the amino acid sequence set forth in any of SEQ ID NOs: 18, 20, 22, 24, 26, 28, 30, 34, 36, 91, 92, 93, 99, 101, and 103; or an amino acid sequence that has at least 80%, 85%, 90%, 91%, 92%, 93%, 94%, 95%, 96%, 97%, 98%, or 99% sequence identity thereto.

Exemplary structure of antigen receptors, including the hinge, the transmembrane domain, and the intracellular T cell signaling domain, and methods for engineering and introducing such receptors into cells, are described, for example, in Chandran et al., “T cell receptor-based cancer immunotherapy: Emerging efficacy and pathways of resistance.” Immunological reviews 290.1 (2019): 127-147; Cartellieri, Marc, et al., “Chimeric antigen receptor-engineered T cells for immunotherapy of cancer.” BioMed Research International 2010 (2010); and PCT publication No. W02017173256A1; US2002/131960, US2013/287748, US2013/0149337, U.S. Pat. Nos. 6,451,995, 7,446,190, 8,252,592; each of which is incorporated herein by reference in its entirety.

The disclosure provides chimeric antigen receptors (CARs) or fragments thereof that specifically bind to ALPP. The CARs or fragments thereof described herein are capable of binding to ALPP.

The disclosure provides CARs or fragments thereof, comprising (a) an extracellular antigen-binding domain that specifically recognizes alkaline phosphatase, placental (ALPP); (b) a transmembrane domain; and (c) an intracellular signaling region. In some embodiments, the antigen-binding domain includes a heavy chain variable domain (VH) and a light chain variable domain (VL). In some embodiments, the VH and VL of the CAR or fragments thereof described herein are identical to the VH and the VL of a mouse anti-ALPP antibody described herein (e.g., A02). In some embodiments, the VH and the VL of the CAR or fragments described herein are identical to the VH and the VL of a humanized anti-ALPP antibody described herein (e.g., A03, A04, A05, A06, A07, or A08).

The CDR sequences of the antigen-binding domain (e.g., an scFv) for A02 CAR, related antibody or antigen binding fragment thereof include VH CDR1, VH CDR2, and VH CDR3 comprising or consisting of SEQ ID NOs: 45, 46, and 47, respectively, and VL CDR1, VL CDR2, and VL CDR3 comprising or consisting of SEQ ID NOs: 48, 49, and 50, respectively.

The CDR sequences of the antigen-binding domain (e.g., an scFv) for A03 CAR, related antibody or antigen binding fragment thereof include VH CDR1, VH CDR2, and VH CDR3 comprising or consisting of SEQ ID NOs: 51, 52, and 53, respectively, and VL CDR1, VL CDR2, and VL CDR3 comprising or consisting of SEQ ID NOs: 54, 55, and 56, respectively.

The CDR sequences of the antigen-binding domain (e.g., an scFv) for A04 CAR, related antibody or antigen binding fragment thereof include VH CDR1, VH CDR2, and VH CDR3 comprising or consisting of SEQ ID NOs: 57, 58, and 59, respectively, and VL CDR1, VL CDR2, and VL CDR3 comprising or consisting of SEQ ID NOs: 60, 61, and 62, respectively.

The CDR sequences of the antigen-binding domain (e.g., an scFv) for A05 CAR, related antibody or antigen binding fragment thereof include VH CDR1, VH CDR2, and VH CDR3 comprising or consisting of SEQ ID NOs: 63, 64, and 65, respectively, and VL CDR1, VL CDR2, and VL CDR3 comprising or consisting of SEQ ID NOs: 66, 67, and 68, respectively.

The CDR sequences of the antigen-binding domain (e.g., an scFv) for A06 CAR, related antibody or antigen binding fragment thereof include VH CDR1, VH CDR2, and VH CDR3 comprising or consisting of SEQ ID NOs: 69, 70, and 71, respectively, and VL CDR1, VL CDR2, and VL CDR3 comprising or consisting of SEQ ID NOs: 72, 73, and 74, respectively.

The CDR sequences of the antigen-binding domain (e.g., an scFv) for A07 CAR, related antibody or antigen binding fragment thereof include VH CDR1, VH CDR2, and VH CDR3 comprising or consisting of SEQ ID NOs: 75, 76, and 77, respectively, and VL CDR1, VL CDR2, and VL CDR3 comprising or consisting of SEQ ID NOs: 78, 79, and 80, respectively.

The CDR sequences of the antigen-binding domain (e.g., an scFv) for A08 CAR, related antibody or antigen binding fragment thereof include VH CDR1, VH CDR2, and VH CDR3 comprising or consisting of SEQ ID NOs: 81, 82, and 83, respectively, and VL CDR1, VL CDR2, and VL CDR3 comprising or consisting of SEQ ID NOs: 84, 85, and 86, respectively.

The amino acid sequence of VH in the antigen-binding domain for A02 CAR, related antibody or antigen binding fragment thereof is set forth in SEQ ID NO: 1. The amino acid sequence of VL in the antigen-binding domain for A02 CAR, related antibody or antigen binding fragment thereof is set forth in SEQ ID NO: 2 or 98.

The amino acid sequence of VH in the antigen-binding domain for A03 CAR, related antibody or antigen binding fragment thereof is set forth in SEQ ID NO: 3. The amino acid sequence of VL in the antigen-binding domain for A03 CAR, related antibody or antigen binding fragment thereof is set forth in SEQ ID NO: 4.

The amino acid sequence of VH in the antigen-binding domain for A04 CAR, related antibody or antigen binding fragment thereof is set forth in SEQ ID NO: 5. The amino acid sequence of VL in the antigen-binding domain for A04 CAR, related antibody or antigen binding fragment thereof is set forth in SEQ ID NO: 6.

The amino acid sequence of VH in the antigen-binding domain for A05 CAR, related antibody or antigen binding fragment thereof is set forth in SEQ ID NO: 7. The amino acid sequence of VL in the antigen-binding domain for A05 CAR, related antibody or antigen binding fragment thereof is set forth in SEQ ID NO: 8.

The amino acid sequence of VH in the antigen-binding domain for A06 CAR, related antibody or antigen binding fragment thereof is set forth in SEQ ID NO: 9. The amino acid sequence of VL in the antigen-binding domain for A06 CAR, related antibody or antigen binding fragment thereof is set forth in SEQ ID NO: 10.

The amino acid sequence of VH in the antigen-binding domain for A07 CAR, related antibody or antigen binding fragment thereof is set forth in SEQ ID NO: 11. The amino acid sequence of VL in the antigen-binding domain for A07 CAR, related antibody or antigen binding fragment thereof is set forth in SEQ ID NO: 12.

The amino acid sequence of VH in the antigen-binding domain for A08 CAR, related antibody or antigen binding fragment thereof is set forth in SEQ ID NO: 13. The amino acid sequence of VL in the antigen-binding domain for A08 CAR, related antibody or antigen binding fragment thereof is set forth in SEQ ID NO: 14.

In some embodiments, the amino acid sequences for VH and VL of the antigen-binding domain for the CAR, related antibody or antigen binding fragment thereof are humanized (e.g., a sequence can be modified with different amino acid substitutions). In some embodiments, the VH and VL can have more than one version of humanized sequences. In some embodiments, the humanized VH is at least 80%, 85%, 90%, 91%, 92%, 93%, 94%, 95%, 96%, 97%, 98%, or 99% identical to SEQ ID NO: 1, 3, 5, 7, 9, 11, or 13. In some embodiments, the humanized VL is at least 80%, 85%, 90%, 91%, 92%, 93%, 94%, 95%, 96%, 97%, 98%, or 99% identical to SEQ ID NO: 2, 4, 6, 8, 10, 12, 14, or 98.

Furthermore, in some embodiments, the CAR, related antibody or antigen binding fragment thereof described herein can also contain one, two, or three heavy chain variable region CDRs selected from the group of SEQ ID NOs: 45-47, SEQ ID NOs: 51-53, SEQ ID NOs: 57-59, SEQ ID NOs: 63-65, SEQ ID NOs: 69-71, SEQ ID NOs: 75-77, and SEQ ID NOs: 81-83; and/or one, two, or three light chain variable region CDRs selected from the group of SEQ ID NOs: 48-50, SEQ ID NOs: 54-56, SEQ ID NOs: 60-62, SEQ ID NOs: 66-68, SEQ ID NOs: 72-74, SEQ ID NOs: 78-80, and SEQ ID NOs: 84-86.

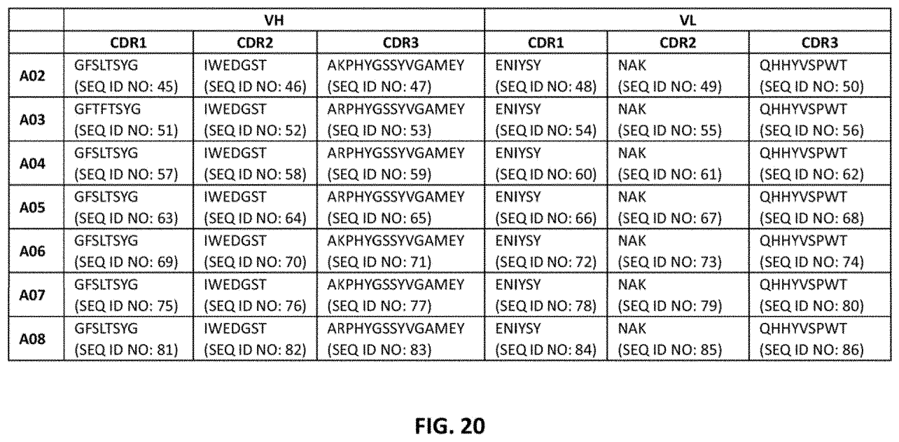

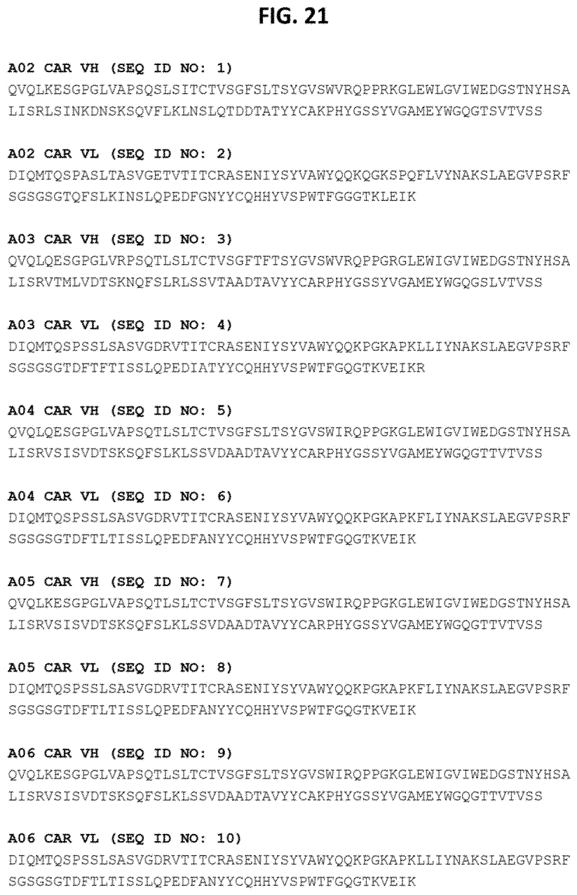

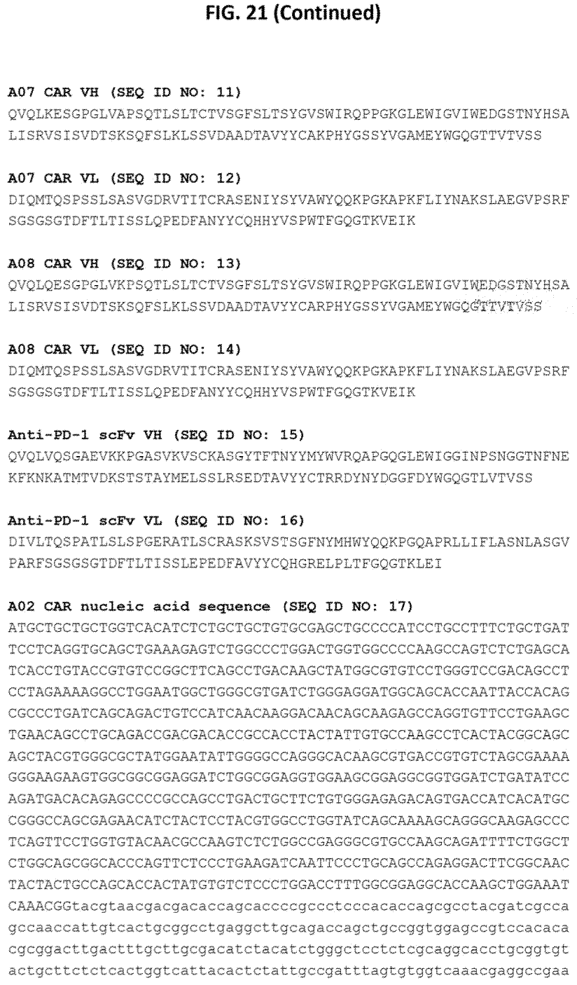

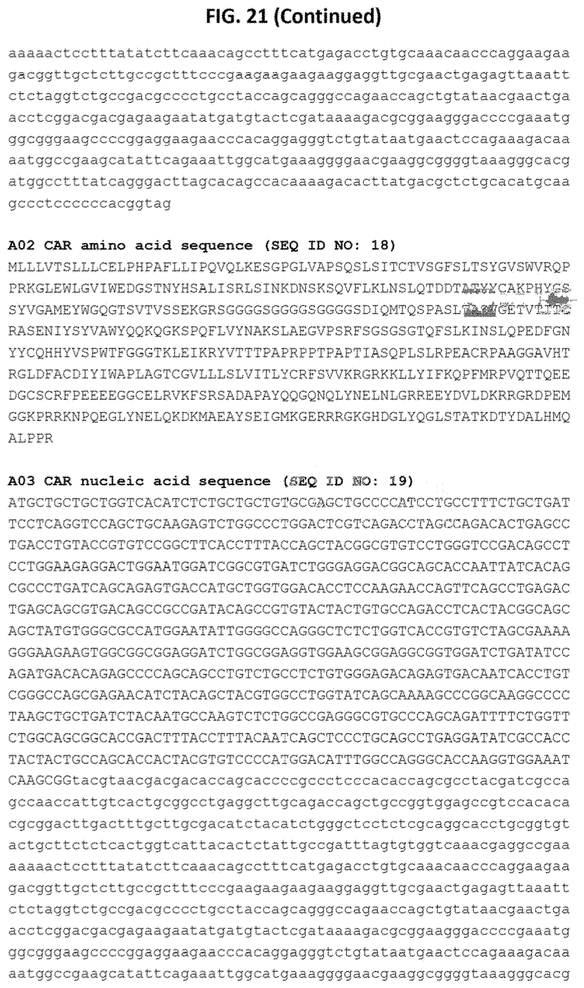

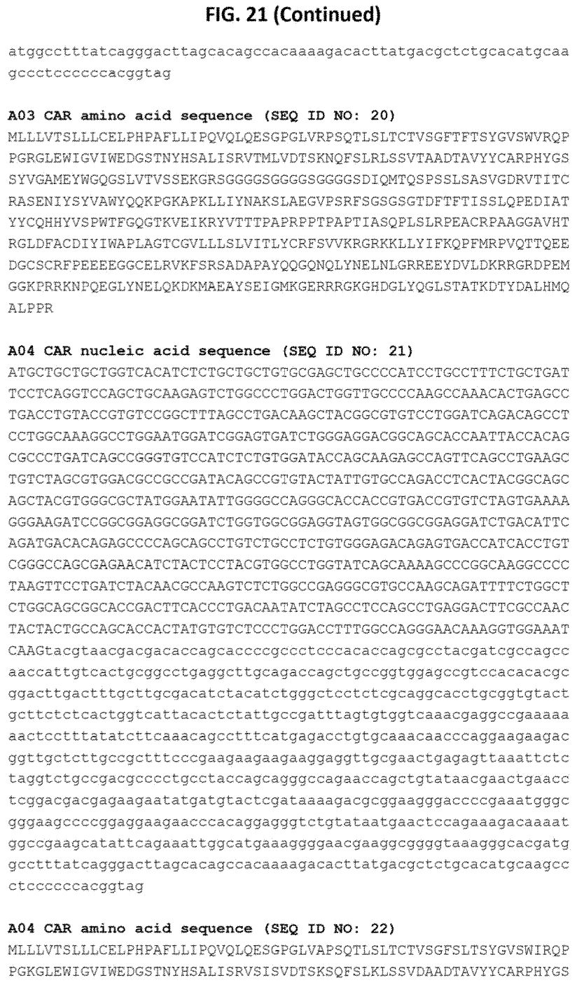

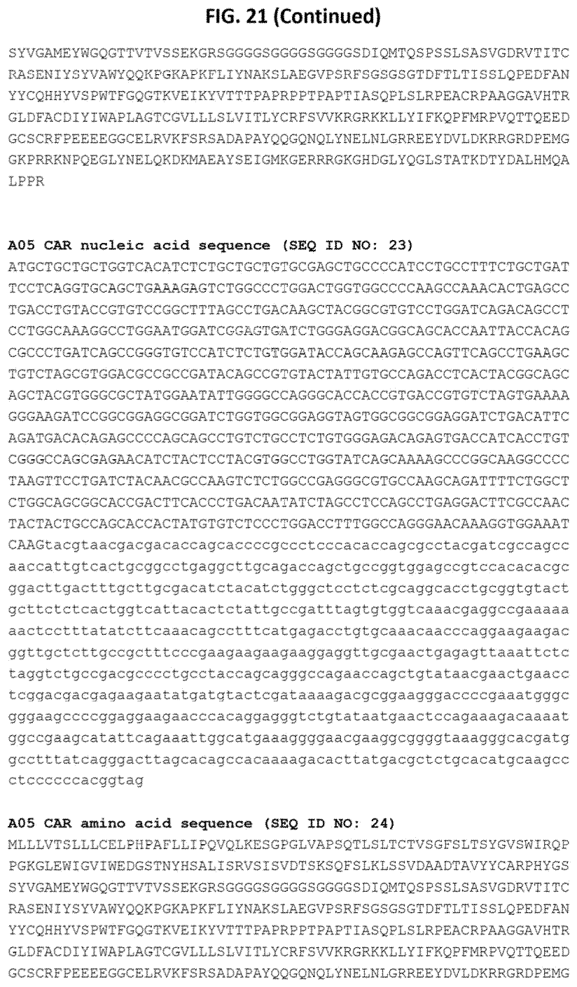

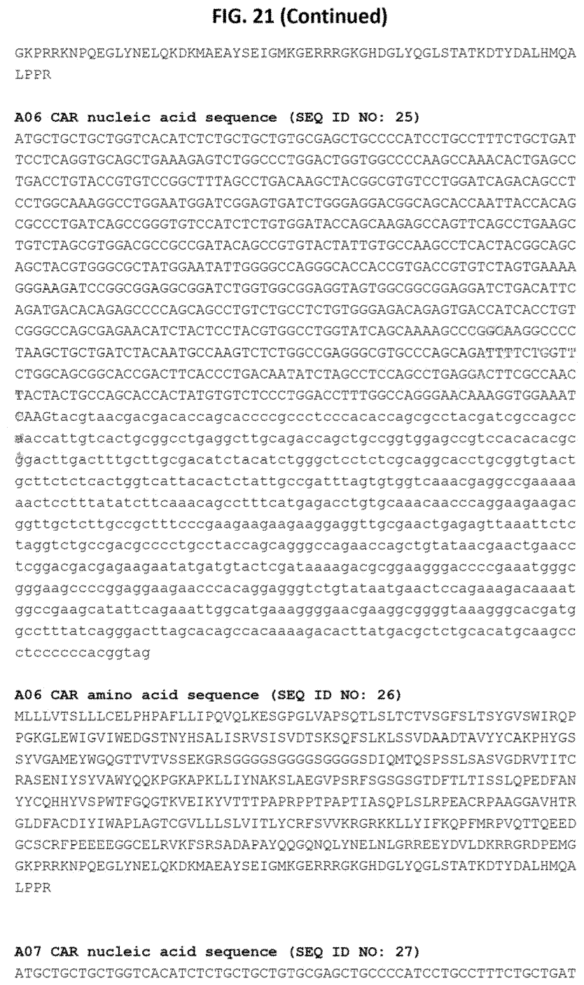

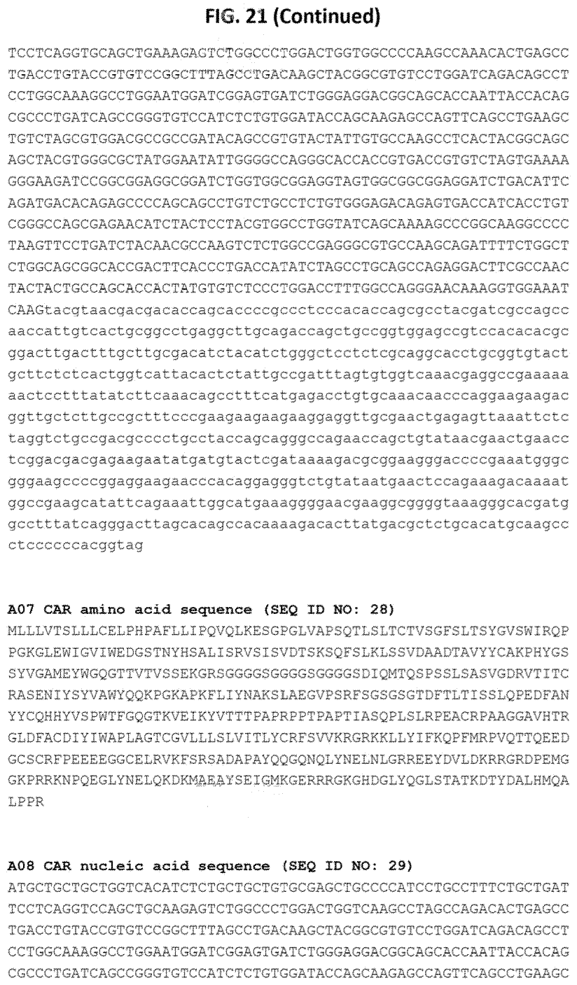

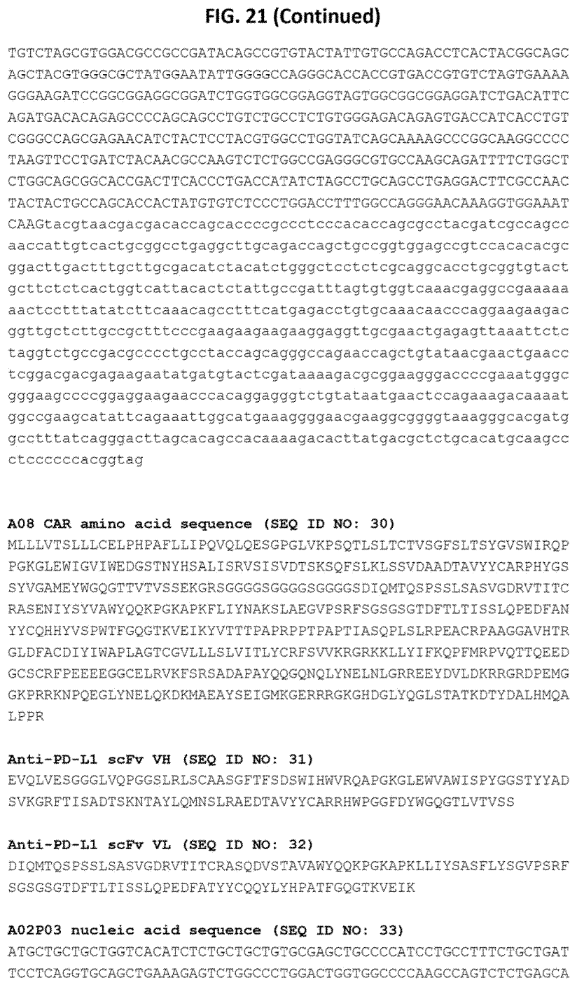

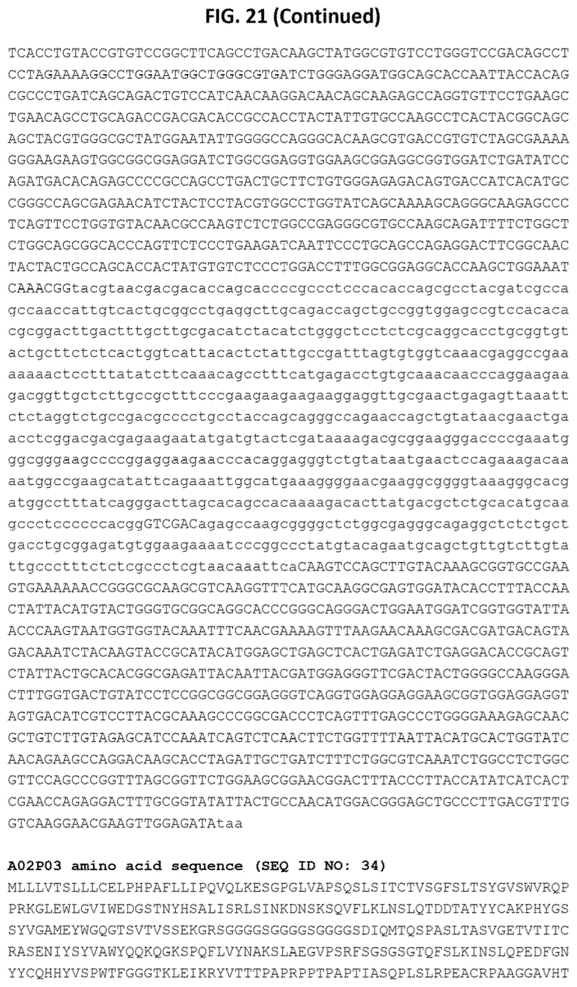

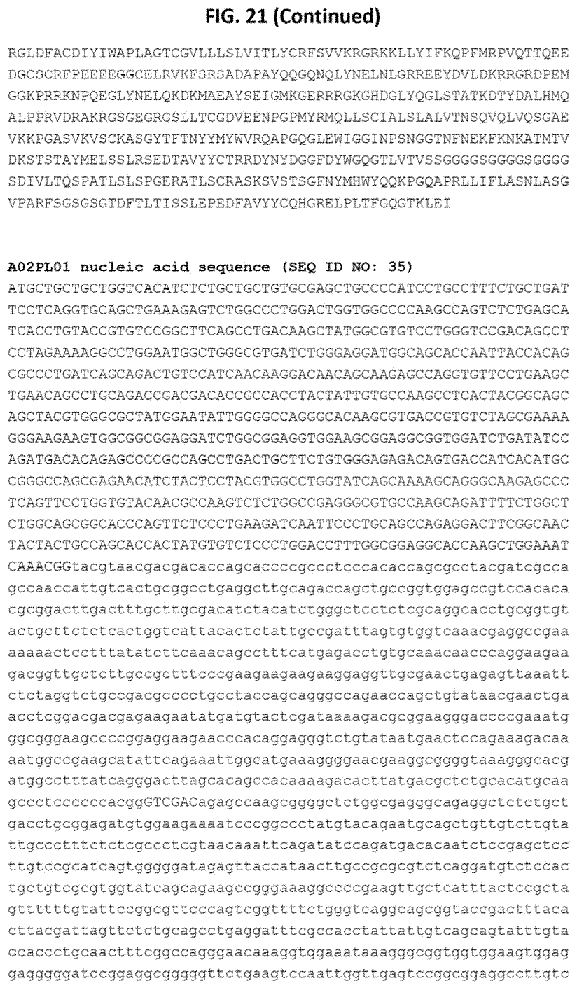

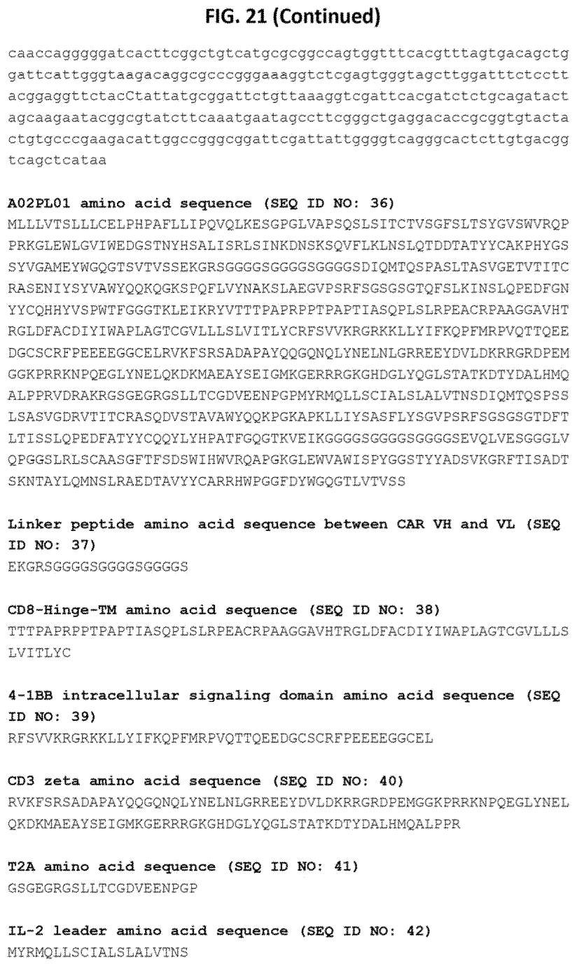

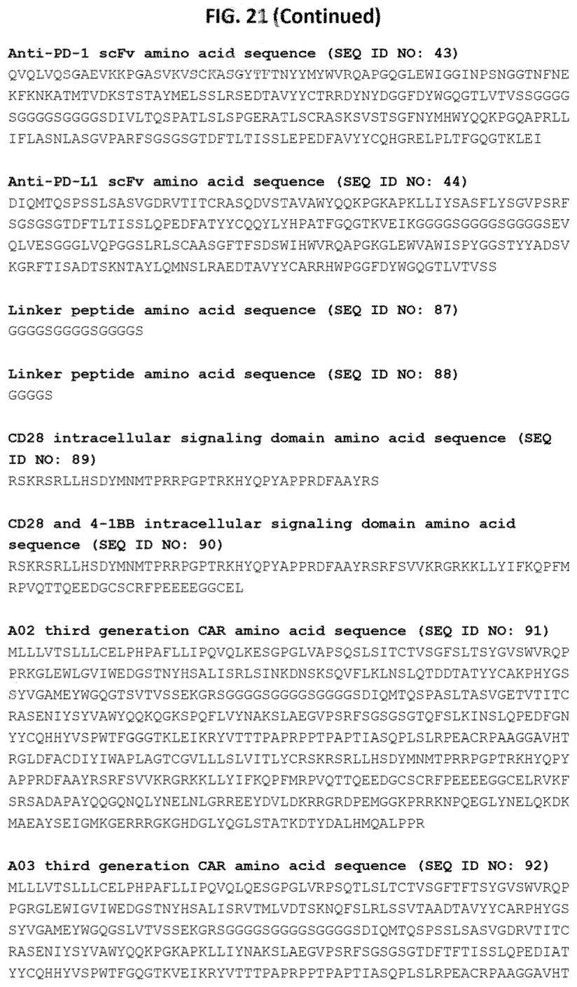

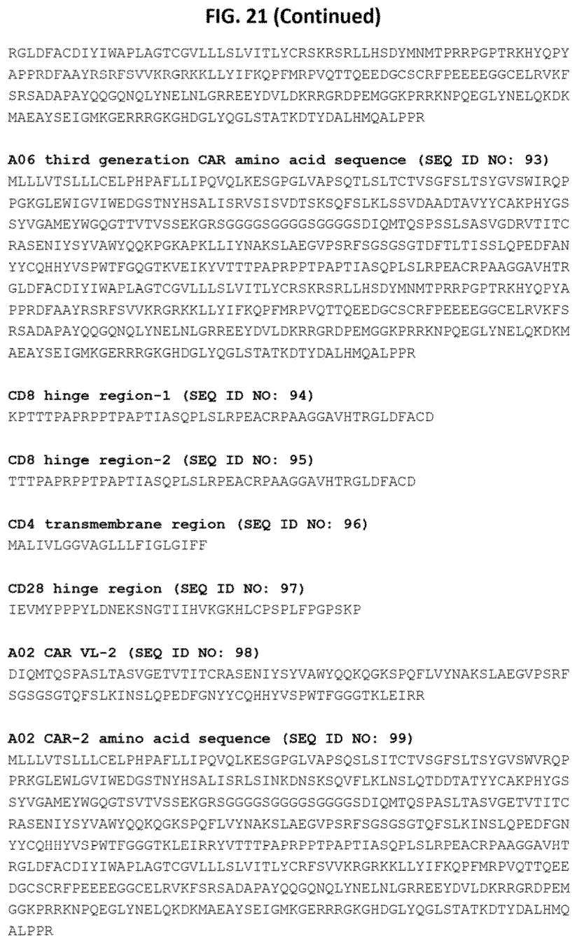

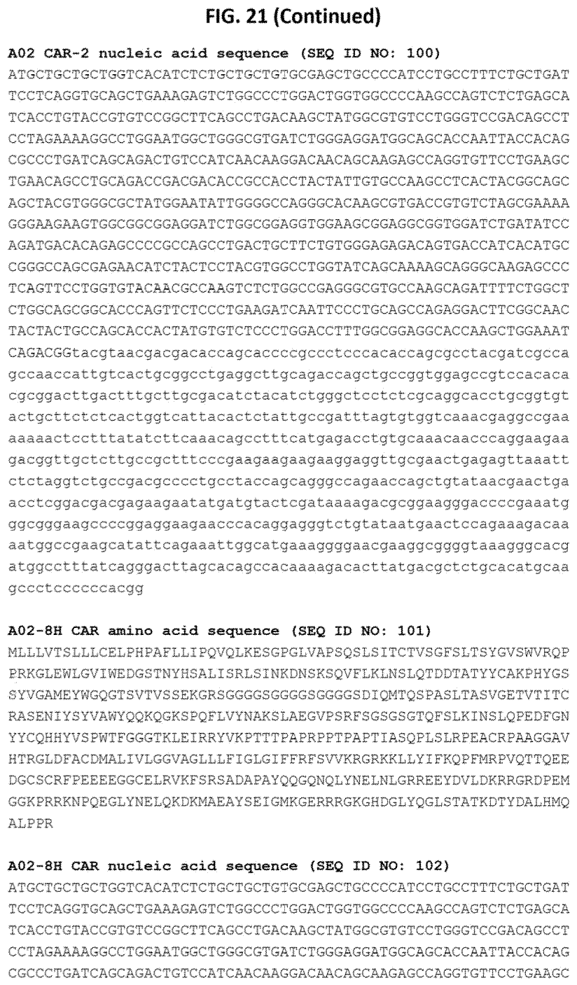

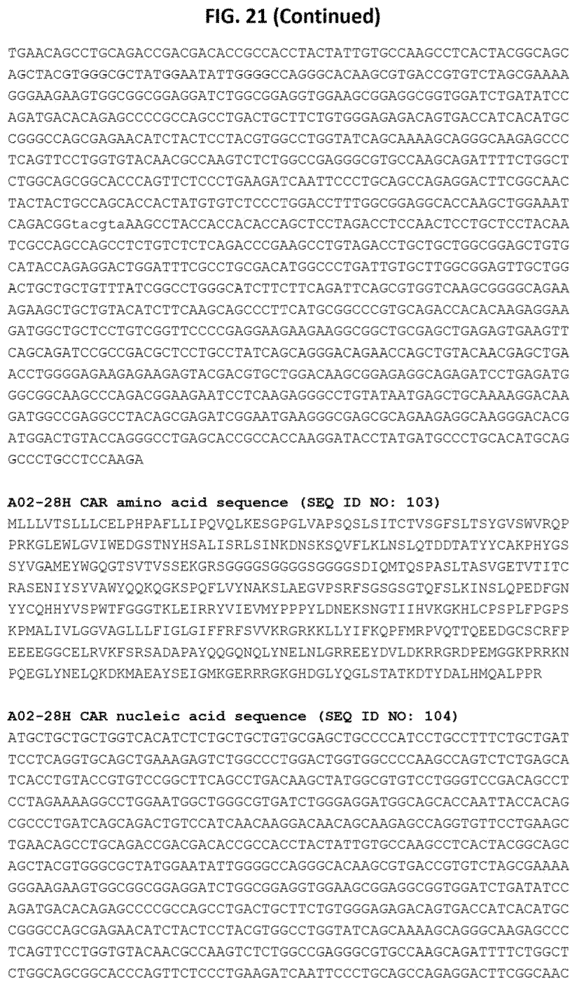

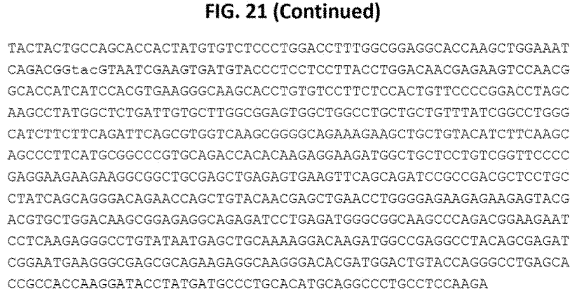

In some embodiments, the CAR, related antibody or antigen binding fragment thereof described herein can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the CAR, related antibody or antigen binding fragment thereof can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 20.

In some embodiments, the CAR, related antibody or antigen binding fragment thereof described herein contains a VH containing one, two, or three of the VH CDR1 with zero, one or two amino acid insertions, deletions, or substitutions; VH CDR2 with zero, one or two amino acid insertions, deletions, or substitutions; VH CDR3 with zero, one or two amino acid insertions, deletions, or substitutions.

In some embodiments, the CAR, related antibody or antigen binding fragment thereof described herein contains a VL containing one, two, or three of VL CDR1 with zero, one or two amino acid insertions, deletions, or substitutions; VL CDR2 with zero, one or two amino acid insertions, deletions, or substitutions; VL CDR3 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme.

The disclosure also provides CARs or fragments thereof that bind to ALPP. The CAR, related antibody or antigen binding fragment thereof contains a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 1, and the selected VL sequence is SEQ ID NO: 2 or 98. In some embodiments, the selected VH sequence is SEQ ID NO: 3 and the selected VL sequence is SEQ ID NO: 4. In some embodiments, the selected VH sequence is SEQ ID NO: 5 and the selected VL sequence is SEQ ID NO: 6. In some embodiments, the selected VH sequence is SEQ ID NO: 7 and the selected VL sequence is SEQ ID NO: 8. In some embodiments, the selected VH sequence is SEQ ID NO: 9 and the selected VL sequence is SEQ ID NO: 10. In some embodiments, the selected VH sequence is SEQ ID NO: 11 and the selected VL sequence is SEQ ID NO: 12. In some embodiments, the selected VH sequence is SEQ ID NO: 13 and the selected VL sequence is SEQ ID NO: 14.

The nucleic acid sequence and the encoded amino acid sequence for A02 CAR is set forth in SEQ ID NO: 17 and SEQ ID NO: 18, respectively. The nucleic acid sequence and the encoded amino acid sequence for A02 CAR-2 is set forth in SEQ ID NO: 100 and SEQ ID NO: 99, respectively. The nucleic acid sequence and the encoded amino acid sequence for A02-8H CAR is set forth in SEQ ID NO: 102 and SEQ ID NO: 101, respectively. The nucleic acid sequence and the encoded amino acid sequence for A02-28H CAR is set forth in SEQ ID NO: 104 and SEQ ID NO: 103, respectively.

The nucleic acid sequence and the encoded amino acid sequence for A03 CAR is set forth in SEQ ID NO: 19 and SEQ ID NO: 20, respectively.

The nucleic acid sequence and the encoded amino acid sequence for A04 CAR is set forth in SEQ ID NO: 21 and SEQ ID NO: 22, respectively.

The nucleic acid sequence and the encoded amino acid sequence for A05 CAR is set forth in SEQ ID NO: 23 and SEQ ID NO: 24, respectively.

The nucleic acid sequence and the encoded amino acid sequence for A06 CAR is set forth in SEQ ID NO: 25 and SEQ ID NO: 26, respectively.

The nucleic acid sequence and the encoded amino acid sequence for A07 CAR is set forth in SEQ ID NO: 27 and SEQ ID NO: 28, respectively.

The nucleic acid sequence and the encoded amino acid sequence for A08 CAR is set forth in SEQ ID NO: 29 and SEQ ID NO: 30, respectively.

The amino acid sequence for the third generation A02 CAR is set forth in SEQ ID NO: 91.

The amino acid sequence for the third generation A03 CAR is set forth in SEQ ID NO: 92.

The amino acid sequence for the third generation A06 CAR is set forth in SEQ ID NO: 93.

In some embodiments, provided herein are polypeptides or fragments thereof comprising an amino acid sequence that is at least 90%, 91%, 92%, 93%, 94%, 95%, 96%, 97%, 98%, 99% or 100% identical to SEQ ID NO: 18, 20, 22, 24, 26, 28, 30, 34, 36, 91, 92, 93, 99, 101, or 103. In some embodiments, the polypeptide described herein comprises an amino acid sequence as set forth in SEQ ID NOS: 18, 20, 22, 24, 26, 28, 30, 34, 36, 91, 92, 93, 99, 101, or 103; optionally with about or no more than 0, 1, 2, 3, 4, 5, 6, 7, 8, 9, 10, 15, 20, 25, 30, 35, 40, 45, or 50 amino acid insertions, deletions, or substitutions.

In some embodiments, the antigen-binding domain described herein comprises an scFv. In some embodiments, the VH and the VL described herein are joined by a flexible linker. In some embodiments, the flexible linker comprises an amino acid sequence of EKGRSGGGGSGGGGSGGGGS (SEQ ID NO: 37). In some embodiments, the flexible linker comprises an amino acid sequence of GGGGSGGGGSGGGGS (SEQ ID NO: 87). In some embodiments, the flexible linker comprises at least 1, 2, 3, 4, 5, or 6 repeats of GGGGS (SEQ ID NO: 88). In some embodiments, the flexible linker comprises 1, 2, 3, 4, or 5 amino acid insertions, deletions, or substitutions.

In some embodiments, the chimeric antigen receptors (CARs) or fragments thereof described herein comprises a hinge region. In some embodiments, the hinge region is a membrane-proximal region from IgG, CD8, CD28, or any combination thereof. In some embodiments, the hinge region is a membrane-proximal region of CD8 (e.g., human CD8). In some embodiments, the hinge region is a membrane-proximal region of CD28 (e.g., human CD28). In some embodiments, the chimeric antigen receptors (CARs) or fragments thereof described herein comprises a transmembrane region. In some embodiments, the transmembrane domain is a transmembrane domain of 4-1BB/CD137, an alpha chain of a T cell receptor, a beta chain of a T cell receptor, CD3 epsilon, CD4, CD5, CD8, CD8 alpha, CD9, CD16, CD19, CD22, CD28, CD33, CD37, CD45, CD64, CD80, CD86, CD134, CD137, CD154, or a zeta chain of a T cell receptor, or any combination thereof. In some embodiments, the transmembrane region is a transmembrane region from CD8 (e.g., human CD8). In some embodiments, the transmembrane region is a transmembrane region from CD4 (e.g., human CD4). In some embodiments, the hinge region and the transmembrane region are directly joined. In some embodiments, the joined hinge region and the transmembrane region comprises an amino acid sequence that is at least 80%, at least 85%, at least 90%, at least 95%, or 100% identical to SEQ ID NO: 38.

In some embodiments, the chimeric antigen receptors (CARs) or fragments thereof described herein comprises an intracellular signaling region. In some embodiments, the intracellular signaling region comprises an activating cytoplasmic signaling domain, which is capable of inducing a primary activation signal in an immune cell (e.g., a T cell). In some embodiments, the activating cytoplasmic signaling domain is a T cell receptor (TCR) component. In some embodiments, the activating cytoplasmic signaling domain comprises an immunoreceptor tyrosine-based activation motif (ITAM). In some embodiments, the intracellular signaling region comprises an amino acid sequence derived from CD3 zeta, FcR gamma, FcR beta, CD3 gamma, CD3 delta, CD3 epsilon, CD5, CD22, CD79a, CD79b, CD278 (ICOS), FceRI, CD66d, DAP10, DAP12, or combinations thereof. In some embodiments, the intracellular signaling region comprises a functional signaling domain of CD3 zeta (e.g., a human CD3 zeta). In some embodiments, the intracellular signaling region comprises an amino acid sequence that is at least 80%, at least 85%, at least 90%, at least 95%, or 100% identical to SEQ ID NO: 40.

In some embodiments, the chimeric antigen receptors (CARs) or fragments thereof described herein comprises a costimulatory signaling region. In some embodiments, the costimulatory signaling region is between the transmembrane domain and the intracellular signaling region. In some embodiments, the costimulatory signaling region comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a CD83 ligand. In some embodiments, the costimulatory signaling region comprises a functional signaling domain from OX40, CD28, 4-1BB, ICOS, or a signaling portion thereof. In some embodiments, the costimulatory signaling region comprises an intracellular signaling domain of 4-1BB (e.g., human 4-1BB). In some embodiments, the costimulatory signaling region comprises an intracellular signaling domain of CD28 (e.g., human CD28). In some embodiments, the costimulatory signaling region comprises intracellular signaling domains of both CD28 (e.g., human CD28) and 4-1BB (e.g., human 4-1BB).

In some embodiments, the 4-1BB intracellular signaling domain comprises an amino acid sequence that is at least 80%, at least 85%, at least 90%, at least 95%, or 100% identical to SEQ ID NO: 39. In some embodiments, the CD28 intracellular signaling domain comprises an amino acid sequence that is at least 80%, at least 85%, at least 90%, at least 95%, or 100% identical to SEQ ID NO: 89. In some embodiments, the costimulatory signaling region comprises an amino acid sequence that is at least 80%, at least 85%, at least 90%, at least 95%, or 100% identical to SEQ ID NO: 39. In some embodiments, the costimulatory signaling region comprises an amino acid sequence that is at least 80%, at least 85%, at least 90%, at least 95%, or 100% identical to SEQ ID NO: 90.

### Anti-ALPP Antibodies, Antigen-Binding Fragments, and Antigen-Binding Domains

The disclosure provides antibodies and antigen-binding fragments thereof that specifically bind to ALPP. The antibodies and antigen-binding fragments described herein are capable of binding to ALPP. The antigen binding domains of the CARs or fragments thereof described herein can be derived from these antibodies or antigen binding fragments thereof.

The disclosure provides e.g., mouse anti-ALPP antibodies (e.g., A02 or H17E2 antibody), the chimeric antibodies thereof, and the humanized antibodies thereof (e.g., A03, A04, A05, A06, A07, A08, or A09 antibody). The CDR sequences for A02, A03, A04, A05, A06, A07, A08 antibodies, and the derived antibodies or antigen-binding fragments thereof are provided in FIG. 20.

The amino acid sequences for heavy chain variable regions and light variable regions of the humanized antibodies are also provided. As there are different ways to humanize a mouse antibody (e.g., a sequence can be modified with different amino acid substitutions), the heavy chain and the light chain of an antibody can have more than one version of humanized sequences. In some embodiments, the heavy chain variable region is at least 80%, 85%, 90%, 91%, 92%, 93%, 94%, 95%, 96%, 97%, 98%, or 99% identical to SEQ ID NO: 1, 3, 5, 7, 9, 11, or 13. In some embodiments, the light chain variable region is at least 80%, 85%, 90%, 91%, 92%, 93%, 94%, 95%, 96%, 97%, 98%, or 99% identical to SEQ ID NO: 2, 4, 6, 8, 10, 12, 14, or 98. The heavy chain variable region sequence can be paired with the light chain variable region sequence, and together they bind to ALPP.

Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al., “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

Furthermore, in some embodiments, the antibodies or antigen-binding fragments thereof described herein can also contain one, two, or three heavy chain variable region CDRs selected from the group of SEQ ID NOs: 45-47, SEQ ID NOs: 51-53, SEQ ID NOs: 57-59, SEQ ID NOs: 63-65, SEQ ID NOs: 69-71, SEQ ID NOs: 75-77, and SEQ ID NOs: 81-83; and/or one, two, or three light chain variable region CDRs selected from the group of SEQ ID NOs: 48-50, SEQ ID NOs: 54-56, SEQ ID NOs: 60-62, SEQ ID NOs: 66-68, SEQ ID NOs: 72-74, SEQ ID NOs: 78-80, and SEQ ID NOs: 84-86.

The disclosure also provides antibodies or antigen-binding fragments thereof that bind to ALPP. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 1, and the selected VL sequence is SEQ ID NO: 2 or 98. In some embodiments, the selected VH sequence is SEQ ID NO: 3 and the selected VL sequence is SEQ ID NO: 4. In some embodiments, the selected VH sequence is SEQ ID NO: 5 and the selected VL sequence is SEQ ID NO: 6. In some embodiments, the selected VH sequence is SEQ ID NO: 7 and the selected VL sequence is SEQ ID NO: 8. In some embodiments, the selected VH sequence is SEQ ID NO: 9 and the selected VL sequence is SEQ ID NO: 10. In some embodiments, the selected VH sequence is SEQ ID NO: 11 and the selected VL sequence is SEQ ID NO: 12. In some embodiments, the selected VH sequence is SEQ ID NO: 13 and the selected VL sequence is SEQ ID NO: 14.

The anti-ALPP antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgAl, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to ALPP will retain an ability to bind to ALPP. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site. Single-chain Fv or (scFv) antibody fragments comprise the VH and VL domains (or regions) of antibody, wherein these domains are present in a single polypeptide chain. Generally, the scFv polypeptide further comprises a polypeptide linker (e.g., the flexible linker described herein) between the VH and VL domains, which enables the scFv to form the desired structure for antigen binding.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

In one aspect, this disclosure provides an anti-ALPP antibody, or antigen-binding fragment thereof (e.g., scFv) comprising: (a) a light chain variable region comprising an amino acid sequence comprising VL selected from SEQ ID NOs: 2, 4, 6, 8, 10, 12, 14, and 98; and (b) a heavy chain variable region comprising an amino acid sequence comprising VH selected from SEQ ID NOs: 1, 3, 5, 7, 9, 11, and 13; wherein the antibody or antigen-binding fragment thereof specifically binds to ALPP (e.g., endogenous ALPP).

In some embodiments, this disclosure provides an anti-PD-1 antibody, or antigen-binding fragment thereof (e.g., scFv) comprising: (a) a light chain variable region comprising an amino acid sequence comprising VL of SEQ ID NO: 16; and (b) a heavy chain variable region comprising an amino acid sequence comprising VH of SEQ ID NO: 15; wherein the antibody or antigen-binding fragment thereof blocks the interaction between PD-1 and PD-L1.

In some embodiments, this disclosure provides an anti-PD-L1 antibody, or antigen-binding fragment thereof (e.g., scFv) comprising: (a) a light chain variable region comprising an amino acid sequence comprising VL of SEQ ID NO: 32; and (b) a heavy chain variable region comprising an amino acid sequence comprising VH of SEQ ID NO: 31; wherein the antibody or antigen-binding fragment thereof blocks the interaction between PD-1 and PD-L1.

In some embodiments, the VH and VL are joined by a flexible linker. In some embodiments, the VH and VL are joined in an order of VH-flexible linker-VL. In some embodiments, the VH and VL are joined in an order of VL-flexible linker-VH. In some embodiments, the flexible linker comprises an amino acid sequence of GGGGSGGGGSGGGGS (SEQ ID NO: 87). In some embodiments, the flexible linker comprises at least 1, 2, 3, 4, 5, or 6 repeats of GGGGS (SEQ ID NO: 88). In some embodiments, the PD-1 or PD-L1 antibody or antigen binding fragment thereof (e.g., scFv) is linked to a leader peptide. In some embodiments, the leader peptide is a secretion signal peptide. In some embodiments, the leader peptide is a human IL-2 leader peptide (SEQ ID NO: 42).

The present disclosure provides various antibodies and antigen-binding fragments thereof derived from antibodies described herein. In general, antibodies (also called immunoglobulins) are made up of two classes of polypeptide chains, light chains and heavy chains. A non-limiting examples of antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgEl, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the antigen binding surface of the antibody. The four framework regions largely adopt a beta-sheet conformation and the CDRs form loops connecting the beta-sheet structure, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (Oct. 1997); Morea et al., J Mol Biol. 275(2):269-94 (Jan .1998); Chothia et al., Nature 342(6252):877-83 (Dec. 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., an scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

Fragments of antibodies are suitable for use in the methods described herein are also provided. The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, the antibodies or antigen binding fragments can enhance APC (e.g., DC cell) function, for example, inducing surface expression of costimulatory and MHC molecules, inducing production of proinflammatory cytokines, and/or enhancing T cell triggering function.

In some embodiments, the Fc region is human IgG1, human IgG2, human IgG3, or human IgG4. In some embodiments, the antibody is a human IgG1 antibody.

In some embodiments, the antibodies or antigen binding fragments do not have a functional Fc region. For example, the antibodies or antigen binding fragments are Fab, Fab′, F(ab′)2, and Fv fragments. In some embodiments, the Fc region has LALA mutations (L234A and L235A mutations in EU numbering), or LALA-PG mutations (L234A, L235A, P329G mutations in EU numbering).

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta endodomain. In some embodiments, the scFv has one heavy chain variable domain, and one light chain variable domain. In some embodiments, the scFv has two heavy chain variable domains, and two light chain variable domains. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

### CAR, Antibody, Antigen-Binding Fragment Characteristics

In some embodiments, the CAR, antibodies or antigen-binding fragments thereof as described herein can increase immune response, activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds.

In some implementations, the antibody (antigen-binding fragments thereof, or molecules derived therefrom, e.g., CAR) specifically binds to ALPP with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD (Kd) is less than 1×10⁻⁶M, less than 1×10⁻⁷ M, less than 1×10⁻⁸ M, less than 1×10⁻⁹ M, or less than 1×10⁻¹⁰ M. In some embodiments, the KD is less than 50nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., ELISA, RIA, and surface plasmon resonance (SPR). In some embodiments, the antibody binds to human ALPP.

In some embodiments, an antibody of the disclosure binds to a ALPP protein with an EC₅₀ of 0.5 μg/ml or less, binds to a ALPP protein with a EC₅₀ of 0.4 μg/ml or less, binds to a ALPP protein with a EC₅₀ of 0.3 μg/ml or less, binds to a ALPP protein with a EC₅₀ of 0.2 μg/ml or less, binds to a ALPP protein with a EC₅₀ of 0.1 μg/ml or less, binds to a ALPP protein with a EC₅₀ of 0.02 μg/ml or less, binds to a ALPP protein with a EC₅₀ of 0.01 μg/ml or less, binds to a ALPP protein with a EC₅₀ of 0.005 μg/ml or less, binds to a ALPP protein with a EC₅₀ of 0.004 μg/ml or less, binds to a ALPP protein with a EC₅₀ of 0.003 μg/ml or less, binds to a ALPP protein with a EC₅₀ of 0.0.002 μg/ml or less, binds to a ALPP protein with a EC₅₀ of 0.001 μg/ml or less. EC₅₀ can be determined by methods described herein, e.g., ELISA or cell flow cytometry.

In some embodiments, an ALPP CAR described herein having a CD28 hinge region can be more stably expressed on the T cell surface than the same ALPP CAR but having a CD8 hinge region. In some embodiments, an ALPP CAR described herein having a CD4 transmembrane region can be more stably expressed on the T cell surface than the same ALPP CAR but having a CD8 transmembrane region.

### Engineered Cells

The present disclosure provides engineered cells (e.g., immune cells, T cells, NK cells, tumor-infiltrating lymphocytes) that express CAR, and/or various proteins as described herein. These engineered cells can be used to treat various disorders or disease as described herein (e.g., ALPP-associated cancer).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. In some embodiments, the cells are mouse lymphocytes and engineered (e.g., transduced) to express the CAR, or antigen-binding fragment thereof. In some embodiments, the cell is obtained from humans. In various embodiments, the cell that is engineered is a blood cell. Preferably, the cell is a leukocyte (e.g., a T cell), lymphocyte or any other suitable blood cell type. In some embodiments, the cell is a peripheral blood cell. In some embodiments, the cell is a tumor-infiltrating lymphocyte (TIL). In some embodiments, the cell is a T cell, B cell or NK cell. In some embodiments, the cells are human peripheral blood mononuclear cells (PBMCs). In some embodiments, the human PBMCs are CD3+ cells. In some embodiments, the human PBMCs are CD8+ cells.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. Genetically modified T cells can be obtained by transducing T cells (e.g., isolated from the peripheral blood of patients), with a viral vector. In some embodiments, the T cells are CD4+ T cells, CD8+ T cells, or regulatory T cells. In some embodiments, the T cells are T helper type 1 T cells and T helper type 2 T cells. In some embodiments, the T cell expressing this receptor is an αβ-T cell. In alternate embodiments, the T cell expressing this receptor is a γδ-T cell. In some embodiments, the T cells are central memory T cells. In some embodiments, the T cells are effector memory T cells. In some embodiments, the T cells are naïve T cells.

In some embodiments, the cell is an NK cell. In some embodiments, preparation of the engineered cells includes one or more culture and/or preparation steps. The cells for introduction of the binding molecule, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are stem cells, such as multipotent and pluripotent stem cells, including induced pluripotent stem cells (iPSCs). The cells can be primary cells, such as those isolated directly from a subject and/or isolated from a subject and frozen. In some embodiments, the stem cells are cultured with additional differentiation factors to obtain desired cell types (e.g., T cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g. CAR, e.g. TCR-like CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety.

Also provided are populations of engineered cells, compositions containing such cells and/or enriched for such cells, such as in which cells expressing the binding molecule make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

In some embodiments, the engineered cells (e.g. CAR-T cells) are co-cultured with target cells (e.g., antigen presenting cells) for at least or about 1 hour, 2 hours, 3 hours, 4 hours, 5 hours, 6 hours, 12 hours, 18 hours, 1 day, 2 days, 3 days, or longer, such that the engineered cells (e.g., CAR-T cells) can be activated. In some embodiments, the target cells are Jurkat cells or SiHa cells.

In some embodiments, IL-12 and modified IL-12 can be expressed by the engineered cells. For example, the fusion protein comprising the modified IL-12 described herein can be expressed on cell surface of engineered cells, e.g., when the fusion protein is a membrane-tethered protein. In some instances, the fusion protein comprising modified IL-12 described herein can be expressed and secreted, e.g., when the fusion protein is a soluble protein. The expression of IL-12 in the engineered cells provides some additional benefits. For example, it can increase production of IFN-γ, which is the most potent mediator of IL-12 actions, from NK and T cells, stimulate of growth and cytotoxicity of activated NK cells, CD8+ and CD4+ T cells, shift differentiation of CD4+ Th0 cells toward the Thl phenotype, increase antibody-dependent cellular cytotoxicity (ADCC) against tumor cells, and induce IgG and suppression of IgE production from B cells, e.g., by at least or about 1 fold, 2 folds, 3 folds, 4 folds, 5 folds, 10 folds, or 20 folds.

In some embodiments, co-culturing with the target cells can increase cytokine (e.g., IFN-γ) secretion of the engineered cells by at least or about 1 fold, 2 folds, 5 folds, 10 folds, 20 folds, 30 folds, 40 folds, 50 folds, 60 folds, 70 folds, 80 folds, 90 folds, 100 folds, 200 folds, 500 folds, 1000 folds, 2000 folds, 5000 folds, 10000 folds, or more as compared to the cytokine secretion level of the engineered cell without co-culturing.

In some embodiments, the cells are human PBMCs and engineered (e.g., transduced) to express the CAR, or antigen-binding fragment thereof.

In some embodiments, when the engineered cells are co-cultured with target cells (e.g., ALPP expressing cells), the engineered cells can increase cytokine (e.g., IFN-γ) expression or secretion by at least or about 10%, 20%, 30%, 40%, 50%, 60%, 70%, 80%, 90%, 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, or more. In some embodiments, when the engineered cells are co-cultured with target cells (e.g., ALPP expressing cells), the activated T cell population is increased by at least or about 10%, 20%, 30%, 40%, 50%, 60%, 70%, 80%, 90%, 1 fold, 2 folds, 3 folds, 4 folds, 5 folds, 10 folds, 20 folds, 50 folds, 100 folds, or more. In some embodiments, the T cell activation status can be measured by CD69 expression levels.

In some embodiments, provided herein is an ALPP CAR having a CD28 hinge region and/or a CD4 transmembrane region. In some embodiments, CAR-T cells expressing a CAR having a CD28 hinge region and/or a CD4 transmembrane region can secret less than 70%, less than 60%, less than 50%, less than 40%, less than 30%, less than 20%, less than 10%, less than 5%, less than 3%, less than 2%, or less than 1% of a pro-inflammatory cytokine (e.g., IFN-γ) than CAR-T cells expressing the same CAR but having a CD8 hinge region and CD8 transmembrane region.

In some embodiments, CAR-T cells expressing a CAR having a CD28 hinge region and/or a CD4 transmembrane region can proliferate at an expansion ratio that is at least 10%, at least 20%, at least 30%, at least 40%, at least 50% higher than that of CAR-T cells expressing the same CAR but having a CD8 hinge region and CD8 transmembrane region.

In some embodiments, upon co-culture with target tumor cells (e.g., SiHa), CAR-T cells expressing a CAR having a CD28 hinge region and/or a CD4 transmembrane region can increase intracellular IFN-γ expression by at least 5%, at least 10%, at least 20%, at least 30%, at least 40%, at least 50%, at least 60%, at least 70%, at least 80%, at least 90%, or at least 100% as compared to CAR-T cells expressing the same CAR but having a CD8 hinge region and CD8 transmembrane region.

In some embodiments, CAR-T cells expressing a CAR having a CD28 hinge region and/or a CD4 transmembrane region have a competitive killing efficacy that is about 80%, about 90%, about 100%, about 110%, or about 120% as compared to that of CAR-T cells expressing the same CAR but having a CD8 hinge region and CD8 transmembrane region, as determined using the methods described herein.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

A vector is a construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

In some embodiments, the vector is a viral vector. The viral vector can be grown in a culture medium specific for viral vector manufacturing. Any suitable growth media and/or supplements for growing viral vectors can be used in accordance with the embodiments described herein. In some embodiments, a MP71 vector is used.

In some embodiments, the vector used is a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. For example, such vectors can include a 5′ LTR, a leading sequence, a tRNA binding site, a packaging signal, an origin of second strand DNA synthesis, and a 3′ LTR or a portion thereof. Additionally, retroviral vector used herein can also refers to the recombinant vectors created by removal of the retroviral gag, pol, and env genes and replaced with the gene of interest.

In some embodiments, the vector or construct can contain a single promoter that drives the expression of one or more nucleic acid molecules. In some embodiments, such promoters can be multicistronic (bicistronic or tricistronic). For example, in some embodiments, transcription units can be engineered as a bicistronic unit containing an IRES (internal ribosome entry site), which allows coexpression of gene products (e.g. encoding CAR and an antibody or antigen binding fragment thereof) by a message from a single promoter. Alternatively, in some cases, a single promoter may direct expression of an RNA that contains, in a single open reading frame (ORF), two or three genes (e.g. encoding CAR and/or an antibody or antigen binding fragment thereof) separated from one another by sequences encoding a self-cleavage peptide (e.g., P2A or T2A) or a protease recognition site (e.g., furin). The ORF thus encodes a single polyprotein, which, either during (in the case of 2A e.g., T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream.

Various cell lines can be used in connection with the vectors as described herein. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, a particular eukaryotic host cell is selected based on its ability to make desired post-translational modifications to the binding molecule. For example, in some embodiments, CHO cells produce polypeptides that have a higher level of sialylation than the same polypeptide produced in 293 cells. In one aspect, the disclosure relates to a cell comprising the vector or the pair of vectors as described herein. In some embodiments, the cell is a T cell.

In some embodiments, provided herein are vectors encoding CARs or fragments thereof. In some embodiments, the vectors comprise a nucleic acid sequence that is at least 90%, 91%, 92%, 93%, 94%, 95%, 96%, 97%, 98%, 99% or 100% identical to SEQ ID NOs: 17, 19, 21, 23, 25, 27, 29, 33, 35, 100, 102, or 104. In some embodiments, the vectors encode an amino acid sequence that is at least 90%, 91%, 92%, 93%, 94%, 95%, 96%, 97%, 98%, 99% or 100% identical to SEQ ID NOS: 18, 20, 22, 24, 26, 28, 30, 34, 36, 91, 92, 93, 99, 101, or 103. In some embodiments, sequence of the vectors are codon-optimized.

In one aspect, the disclosure provides a vector comprising a nucleic acid encoding an immune checkpoint (e.g., PD1, PD-L1, PD-L2, CTLA4, TIM3, CEACAM-1, CEACAM-3, CEACAM-5, LAG3, VISTA, BTLA, TIGIT, LAIRI, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGFR, e.g., TGFRbeta) inhibitor. In some embodiments, the immune checkpoint inhibitor is an anti-PD-1 or anti-PD-L1 antibody, or antigen binding fragment thereof. In some embodiments, the vector comprises a nucleic acid encoding an scFv that specifically binds to PD-1 (e.g., human PD-1) or PD-L1 (e.g., human PD-L1). In some embodiments, the scFv comprises a heavy chain variable region (VH) and a light chain variable region (VL), and the VH and VL are joined by a flexible linker. In some embodiments, the VH and VL are joined in an order of VH-flexible linker-VL. In some embodiments, the VH and VL are joined in an order of VL-flexible linker-VH. In some embodiments, the amino acid sequence of the VH of the anti-PD-1 antibody or antigen binding fragment (e.g., scFv) is set forth in SEQ ID NO: 15. In some embodiments, the amino acid sequence of the VL of the anti-PD-1 antibody or antigen binding fragment (e.g., scFv) is set forth in SEQ ID NO: 16. In some embodiments, the amino acid sequence of the VH of the anti-PD-L1 antibody or antigen binding fragment (e.g., scFv) is set forth in SEQ ID NO: 31. In some embodiments, the amino acid sequence of the VL of the anti-PD-1 antibody or antigen binding fragment (e.g., scFv) is set forth in SEQ ID NO: 32. In some embodiments, the flexible linker comprises an amino acid sequence of GGGGSGGGGSGGGGS (SEQ ID NO: 87). In some embodiments, the flexible linker comprises at least 1, 2, 3, 4, 5, or 6 repeats of GGGGS (SEQ ID NO: 88). In some embodiments, the vector further comprises a nucleic acid encoding a leader peptide. In some embodiments, the leader peptide is a human IL-2 leader peptide (SEQ ID NO: 42). In some embodiments, the vector comprises a nucleic acid sequence that is at least 80%, at least 85%, at least 90%, at least 95%, at least 99%, or 100% identical to SEQ ID NO: 33. In some embodiments, the vector comprises a nucleic acid sequence encoding an amino acid sequence that is at least 80%, at least 85%, at least 90%, at least 95%, at least 99%, or 100% identical to SEQ ID NO: 34. In some embodiments, the vector comprises a nucleic acid sequence that is at least 80%, at least 85%, at least 90%, at least 95%, at least 99%, or 100% identical to SEQ ID NO: 35. In some embodiments, the vector comprises a nucleic acid sequence encoding an amino acid sequence that is at least 80%, at least 85%, at least 90%, at least 95%, at least 99%, or 100% identical to SEQ ID NO: 36.

In some embodiments, the vector encodes a fusion polypeptide comprising a CAR (e.g., the A02, A02-8H, A02-28H, A03, A04, A05, A06, A07, or A08 CAR) and an immune checkpoint inhibitor (e.g., an anti-PD-1 or anti-PD-L1 scFv). In some embodiments, the vector further comprises a nucleic acid encoding T2A (SEQ ID NO: 41). In some embodiments, the CAR and the immune checkpoint inhibitor are joined by an amino acid sequence comprising the T2A.

The present disclosure also provides a nucleic acid sequence comprising a nucleotide sequence encoding any of the CAR, antigen binding fragments thereof, and/or CAR-derived binding molecules (including e.g., functional portions and functional variants thereof, polypeptides, or proteins described herein). “Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. In some of any such embodiments, the nucleotide sequence is codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein.

In some embodiments, the nucleotide sequence encoding the alpha chain and the nucleotide sequence encoding the beta chain are separated by a peptide sequence that causes ribosome skipping. In some embodiments, the peptide that causes ribosome skipping is a P2A or T2A peptide. In some embodiments, the nucleic acid is synthetic. In some embodiments, the nucleic acid is cDNA.

In some embodiments, the vector can additionally include a nucleic acid sequence that encodes a checkpoint inhibitor (CPI) (e.g., an inhibitory protein). In some embodiments, the checkpoint inhibitor is e.g., any antibody or antigen binding fragment thereof as described herein. In some embodiments, the antibody or antigen binding fragments thereof can specifically bind to PD-1, PD-L1, PD-L2, 2B4 (CD244), 4-1BB, A2aR, B7.1, B7.2, B7-H2, B7-H3, B7-H4, B7-H6, BTLA, butyrophilins, CD160, CD48, CTLA4, GITR, gp49B, HHLA2, HVEM, ICOS, ILT-2, ILT-4, MR family receptors, LAG-3, OX-40, PIR-B, SIRPalpha (CD47), TFM-4, TIGIT, TIM-1, TIM-3, TIM-4, or VISTA. In some embodiments, the inhibitory protein is an scFv (e.g., an anti-PD-1 scFv). In some embodiments, the vector can additionally include a nucleic acid sequence that encodes a bifunctional trap fusion protein. In some embodiments, the bifunctional trap protein targets both the PD-1 and TGF-β. In some embodiments, the bifunctional trap protein targets both the PD-L1 and TGF-β. In some embodiments, the bifunctional fusion protein designed to block PD-L1 and sequester TGF-β. M7824 (MSB0011395C) comprises the extracellular domain of human TGF-β receptor II (TGFβRII) linked to the C-terminus of the human anti-PD-L1 scFv, based on the human IgG1 monoclonal antibody (mAb) avelumab. In some embodiments, the bifunctional fusion protein comprises the extracellular domain of human TGF-β receptor II (TGFβRII) linked to the C-terminus of the human anti-PD-1 scFv.

In some of any such embodiments, the CAR, or antigen-binding fragment thereof is encoded by a nucleotide sequence that has been codon-optimized. In certain embodiments, the polypeptide comprises a signal peptide. In some embodiments, the polypeptide and/or the fusion protein is recombinant.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

In some embodiments, the nucleic acid sequence is at least or about 10, 20, 30, 40, 50, 60, 70, 80, 90, 100, 110, 120, 130, 150, 200, 250, 300, 350, 400, 500, or 600 nucleotides. In some embodiments, the amino acid sequence is at least or about 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

### Method for Preparation of Engineered Cells

The present disclosure provides a method or process for preparing, manufacturing and/or using the engineered cells for treatment of pathological diseases or conditions.

The cells for introduction of the protein described herein, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, or non-human primate. In some embodiments, the cells are isolated from mouse lymph nodes.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge. In some aspects, a washing step is accomplished by tangential flow filtration (TFF). In some embodiments, the cells are resuspended in a variety of biocompatible buffers after washing, such as, for example, Ca²⁺/Mg²⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the method comprises one or more steps of: e.g., isolating the T cells from a patient's blood; transducing the population T cells with a viral vector including the nucleic acid construct encoding a genetically engineered antigen receptor; expanding the transduced cells in vitro; and/or infusing the expanded cells into the patient, where the engineered T cells will seek and destroy antigen positive tumor cells. In some embodiments, the nucleic acid construct further includes a sequence encoding an inhibitory protein. In some embodiments, these engineered T cells can block PD-1/PD-L1 immunosuppression and strengthen the antitumor immune response. The method further comprises: transfection of T cells with the viral vector containing the nucleic acid construct.

In some embodiments, the methods involve introducing any vectors described herein into a cell in vitro or ex vivo. In some embodiments, the vector is a viral vector and the introducing is carried out by transduction. In some embodiments, the cell is transduced for at least or about 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, or longer. In some embodiments, the methods further involve introducing into the cell one or more agent, wherein each of the one or more agent is independently capable of inducing a genetic disruption of a T cell receptor alpha constant (TRAC) gene and/or a T cell receptor beta constant (TRBC) gene. In some embodiments, the one or more agent is an inhibitory nucleic acid (e.g., siRNA). In some embodiments, the one or more agent is a fusion protein comprising a DNA-targeting protein and a nuclease or an RNA-guided nuclease (e.g., a clustered regularly interspaced short palindromic nucleic acid (CRISPR)-associated nuclease).

The transfection of T cells can be achieved by using any standard method such as calcium phosphate, electroporation, liposomal mediated transfer, microinjection, biolistic particle delivery system, or any other known methods by skilled artisan. In some embodiments, transfection of T cells is performed using the calcium phosphate method.

The present disclosure provides a method to create a personalized anti-tumor immunotherapy. Genetically engineered T cells can be produced from a patient's blood cells. These engineered T cells are then reinfused into the patient as a cellular therapy product.

### Methods of Treatment

The methods disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered cells expressing CAR, antigen binding fragments thereof, to a subject in need thereof (e.g., a subject having, or identified or diagnosed as having, a cancer).

In some embodiments, the subject has ALPP-positive cancer. In some embodiments, the subject has ovarian, cervical, or testicular cancer. In some embodiments, the subject has testicular seminoma, primary intracranial germinoma, epithelial ovarian carcinoma, ovarian adenocarcinoma, serous cystadenocarcinoma, undifferentiated carcinoma, dysgerminoma, ovarian cancer, uterus cancer, endometrial cancer, cervical cancer, urothelial cancer, stomach cancer, lung cancer, pancreatic cancer, testis cancer, osteosarcoma, or gastric cancer.

In some embodiments, the IL-12 (e.g., modified IL-12) expressed by the engineered cells described herein can provide improvement (e.g., killing cancer cells, or reducing tumor volume) of treating the heterogeneous cancer by at least or about 10%, 20%, 30%, 40%, 50%, 60%, 70%, 80%, 90%, 100%, 2 folds, 3 folds, 5 folds, 10 folds, 20 folds, 30 folds, 40 folds, 50 folds, 60 folds, 70 folds, 80 folds, 90 folds, 100 folds, or more as compared to similar engineered cells that do not express the IL-12.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

As used herein, the term “delaying development of a disease” refers to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, can be delayed.

An effective amount can be administered in one or more administrations. By way of example, an effective amount of a composition is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, patient history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

In some aspects, the present disclosure also provides methods of diagnosing a disease/condition in a mammal, wherein the CARs, antibodies, or antigen binding fragments, interact with the sample(s) obtained from a subject to form a complex, wherein the sample can comprise one more cells, polypeptides, proteins, nucleic acids, antibodies, or antigen binding portions, blood, whole cells, lysates thereof, or a fraction of the whole cell lysates, e.g., a nuclear or cytoplasmic fraction, a whole protein fraction, or a nucleic acid fraction thereof, wherein the detection of the complex is the indicative of presence of a condition in the mammal, wherein the condition is cancer or infection. Further, the detection of the complex can be in any number of way known in the art but not limited to, ELISA, Flow cytometery, Fluorescence in situ hybridization (FISH), Polymerase chain reaction (PCR), microarray, southern blotting, electrophoresis, Phage analysis, chromatography and more. Thus, the treatment methods can further include determining whether a subject can benefit from a treatment as disclosed herein, e.g., by determining whether the subject has infection or cancer.

In any of the methods described herein, the engineered cells and, and/or at least one additional therapeutic agent can be administered to the subject at least once a week (e.g., once a week, twice a week, three times a week, four times a week, once a day, twice a day, or three times a day). In some embodiments, at least two different engineered cells (e.g., cells express different binding molecules) are administered in the same composition (e.g., a liquid composition). In some embodiments, engineered cells and at least one additional therapeutic agent are administered in the same composition (e.g., a liquid composition). In some embodiments, engineered cells and the at least one additional therapeutic agent are administered in two different compositions. In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, concurrently with, or after administering the engineered cells to the subject.

In some embodiments, one or more additional therapeutic agents can be administered to the subject. The additional therapeutic agent can be a checkpoint inhibitor (CPI). In some embodiments, the checkpoint inhibitor is an inhibitory protein, e.g., an antibody or antigen binding fragment thereof. The checkpoint inhibitor can inhibit or block one or more immune checkpoints, including e.g., PD-1, PD-L1, PD-L2, 2B4 (CD244), 4-1BB, A2aR, B7.1, B7.2, B7-H2, B7-H3, B7-H4, B7-H6, BTLA, butyrophilins, CD160, CD48, CTLA4, GITR, gp49B, HHLA2, HVEM, ICOS, ILT-2, ILT-4, MR family receptors, LAG-3, OX-40, PIR-B, SIRPalpha (CD47), TFM-4, TIGIT, TIM-1, TIM-3, TIM-4, VISTA and combinations thereof. In some embodiments, the inhibitory protein blocks PD-1 or PD-L1. In various embodiments, the inhibitory protein comprises an anti-PD-1 scFv or an anti-PD-L1 scFv. The inhibitory protein is capable of leading to reduced expression of PD-1 or PD-L1 and/or inhibiting upregulation of PD-1 or PD-L1 in T cells in the population and/or physically obstructing the formation of the PD-1/PD-L1 complex and subsequent signal transduction. In some embodiments, the inhibitory protein blocks PD-1. In some embodiments, the additional therapeutic agent is an anti-OX40 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody. In some embodiments, the additional therapeutic agent is an anti-CTLA4 antibody (e.g., ipilimumab), an anti-CD20 antibody (e.g., rituximab), an anti-EGFR antibody (e.g., cetuximab), an anti-CD319 antibody (e.g., elotuzumab), or an anti-PD1 antibody (e.g., nivolumab).

In some embodiments, the additional therapeutic agent is a bifunctional trap fusion protein. Bifunctional trap proteins can target both immune checkpoints and TGF-β negative regulatory pathways. In addition to expression of immune checkpoints, the tumor microenvironment contains other immunosuppressive molecules. Of particular interest is the cytokine TGF-β (TGFB), which has multiple functions in cancer. TGF-β prevents proliferation and promotes differentiation and apoptosis of tumor cells early in tumor development. However, during tumor progression, tumor TGF-β insensitivity arises due to the loss of TGF-β receptor expression or mutation to downstream signaling elements. TGF-β then promotes tumor progression through its effects on angiogenesis, induction of epithelial-to-mesenchymal transition (EMT), and immune suppression. High TGF-β serum level and loss of TGF-β receptor (TGFβR) expression on tumors correlates with poor prognosis. TGFβ-targeted therapies have demonstrated limited clinical activity. In some embodiments, the bifunctional trap protein targets both the PD-1 and TGF-β. In some embodiments, the bifunctional trap protein targets both the PD-L1 and TGF-β. In some embodiments, the bifunctional fusion protein designed to block PD-L1 and sequester TGF-β. M7824 (MSB0011395C) comprises the extracellular domain of human TGF-β receptor II (TGFβRII) linked to the C-terminus of the human anti-PD-L1 scFv, based on the human IgG1 monoclonal antibody (mAb) avelumab. In some embodiments, the bifunctional fusion protein comprises the extracellular domain of human TGF-β receptor II (TGFβRII) linked to the C-terminus of the human anti-PD-1 scFv. These bifunctional trap fusion proteins are described e.g., Knudson, et al., “M7824, a novel bifunctional anti-PD-L1/TGFβ Trap fusion protein, promotes anti-tumor efficacy as monotherapy and in combination with vaccine.” Oncoimmunology 7.5 (2018): e1426519, which is incorporated herein by reference in its entirety. In some embodiments, the subject is treated by cells that express CAR or antigen-binding molecules as described herein and one or more bifunctional trap fusion proteins.

In some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat). In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCLS, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject. In some embodiments, the additional therapeutic agent is selected from asparaginase, busulfan, carboplatin, cisplatin, daunorubicin, doxorubicin, fluorouracil, gemcitabine, hydroxyurea, methotrexate, paclitaxel, rituximab, vinblastine, vincristine and/or combinations thereof.

In some embodiments, the CARs or fragments described herein, optionally in combination with one or more immune check point inhibitors (e.g., an anti-PD-1 or anti-PD-L1 antibody), when expressed by effector cells (e.g., T cells), increase competitive killing percentage of target cells (e.g., SiHa cells) by at least at least 10%, at least 20%, at least 30%, at least 40%, at least 50%, at least 60%, at least 70%, at least 80%, at least 90%, at least 100%, at least 2-fold, at least 3-fold, at least 4-fold, at least 5-fold, at least 10-fold, at least 20-fold, at least 50-fold, or more as compared to the competitive killing percentage of target cells using effector cells not expressing the CARs or fragments thereof.

In some embodiments, the CARs or fragments described herein, optionally in combination with one or more immune check point inhibitors (e.g., an anti-PD-1 or anti-PD-L1 antibody) increase survival rate of a subject (e.g., a mouse or a human patient) by at least 10%, at least 20%, at least 30%, at least 40%, at least 50%, at least 60%, at least 70%, at least 80%, at least 90%, at least 100%, or more as compared to a subject which is not administered with the CARs or fragments thereof.

In some embodiments, the CARs or fragments described herein, optionally in combination with one or more immune check point inhibitors (e.g., an anti-PD-1 or anti-PD-L1 antibody), reduce body weight gain of a subject (e.g., a mouse or a human patient) by less than 50%, less than 40%, less than 30%, less than 20%, less than 10%, less than 5%, less than 1% as compared to a subject which is not administered with the CARs or fragments thereof.

### Compositions and Formulations

The present disclosure provides compositions (including pharmaceutical and therapeutic compositions) containing the engineered cells and populations thereof, produced by the methods disclosed herein. Also provided are methods, e.g., therapeutic methods for administrating the engineered cells and compositions thereof to subjects, e.g., patients or animal models (e.g., mice).

Compositions including the engineered cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient. In some embodiments, the composition includes at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for patient. Additionally, a preparation which is in such form as to permit the biological activity of an active ingredient contained therein to be effective, and which contains no additional components which are unacceptably toxic to a subject to which the formulation would be administered.

In some embodiments, the choice of carrier is determined in part by the particular cell (e.g., T cell or NK cell) and/or by the method of administration. A variety of suitable formulations are available. For example, the pharmaceutical composition can contain preservatives. Suitable preservatives can include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the engineered cells, preferably those with activities complementary to the cells, where the respective activities do not adversely affect one another. Such active ingredients are suitably present in combination in amounts that are effective for the purpose intended. Thus, in some embodiments, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions can be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

The compositions in some embodiments are provided as sterile liquid preparations, e.g., isotonic aqueous solutions, suspensions, emulsions, dispersions, or viscous compositions, which can in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum mono stearate and gelatin.

The formulations to be used for in vivo administration are generally sterile. Sterility can be readily accomplished, e.g., by filtration through sterile filtrationmembranes.

The compositions or pharmaceutical compositions as described herein can be included in a container, pack, or dispenser together with instructions for administration.

### Methods of Administration

Provided are also methods of administering the cells, populations, and compositions, and uses of such cells, populations, and compositions to treat or prevent diseases, conditions, and disorders, including cancers. In some embodiments, the methods described herein can reduce the risk of the developing diseases, conditions, and disorders as described herein.

In some embodiments, the cells, populations, and compositions, described herein are administered to a subject or patient having a particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered T cells.

Methods for administration of cells for adoptive cell therapy are known and can be used in connection with the provided methods and compositions. For example, adoptive T cell therapy methods are described, e.g., in U.S. 2003/0170238; U.S. Pat. No. 4,690,915; Rosenberg, “Cell transfer immunotherapy for metastatic solid cancer—what clinicians need to know.” Nature reviews Clinical oncology 8.10 (2011): 577; Themeli et al., “Generation of tumor-targeted human T lymphocytes from induced pluripotent stem cells for cancer therapy.” Nature biotechnology 31.10 (2013): 928; Tsukahara et al.,“CD19 target-engineered T cells accumulate at tumor lesions in human B-cell lymphoma xenograft mouse models.” Biochemical and biophysical research communications 438.1 (2013): 84-89; Davila et al., “CD19 CAR-targeted T cells induce long-term remission and B Cell Aplasia in an immunocompetent mouse model of B cell acute lymphoblastic leukemia.” PloS one 8.4 (2013); each of which is incorporated herein by reference in its entirety.

In some embodiments, the cell therapy, e.g., adoptive T cell therapy, is carried out by autologous transfer, in which the T cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the T cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the cells are administered prophylactically, e.g., to reduce the likelihood of or prevent relapse. In some embodiments, the subject has not received prior treatment with another therapeutic agent.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at least or at least about or at or about 1×10 ⁵ cells/kg, 1.5×10 ⁵ cells/kg, 2×10 ⁵ cells/kg, or 1×10 ⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at least or at least about or at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at least or at least about or at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the cells are administered at or within a certain range of error of, greater than, and/or at least about lx10⁶, about 2.5 ^{x106,} about 5 ×10⁶, about 7.5 ×10⁶, or about 9×10⁶ CD4+ cells, and/or at least about 1 ×10⁶, about 2.5 ×10⁶, about 5 ×10⁶, about 7.5×10⁶, or about 9×10⁶ CD8+ cells, and/or at least about 1×10⁶, about 2.5 ×10⁶, about 5 ×10⁶, about 7.5 ×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 10⁸ and 10¹² or between about 10¹⁰ and 10¹¹ CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges. In some aspects, the CAR described here provides improved expression and activity, thereby providing therapeutic effects even at a low effector to target (E:T) ratio.

Optimal response to therapy can depend on the ability of the engineered recombinant receptors such as CARs, to be consistently and reliably expressed on the surface of the cells and/or bind the target antigen. For example, in some cases, properties of certain recombinant receptors, e.g., CARs, can affect the expression and/or activity of the recombinant receptor, in some cases when expressed in a cell, such as a human T cell, used in cell therapy. In some contexts, the level of expression of particular recombinant receptors, e.g., CARs, can be low, and activity of the engineered cells, such as human T cells, expressing such recombinant receptors, may be limited due to poor expression or poor signaling activity. In some cases, consistency and/or efficiency of expression of the recombinant receptor, and activity of the receptor is limited in certain cells or certain cell populations of available therapeutic approaches. In some cases, a large number of engineered T cells (a high effector to target (E:T) ratio) is required to exhibit functional activity. In some embodiments, the desired ratio (E:T ratio) is between at or about 1:10 and at or about 10:1 (or greater than about 1:10 and less than about 10:1), or between at or about 1:1 and at or about 10:1 (or greater than about 1:1 and less than about 5:1), such as between at or about 2:1 and at or about 10:1. In some embodiments, the E:T ratio is greater than or about 1:1, 2:1, 3:1, 4:1, 5:1, 6:1, 7:1, 8:1, 9:1, or 10:1.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents.

In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of engineered T cells to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., “Construction and pre-clinical evaluation of an anti-CD19 chimeric antigen receptor.” Journal of immunotherapy (Hagerstown, Md.: 1997) 32.7 (2009): 689 and Hermans et al., “The VITAL assay: a versatile fluorometric technique for assessing CTL-and NKT-mediated cytotoxicity against multiple targets in vitro and in vivo.” Journal of immunological methods 285.1 (2004): 25-40. In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

Repeated dosing methods are provided in which a first dose of cells is given followed by one or more second consecutive doses. The timing and size of the multiple doses of cells generally are designed to increase the efficacy and/or activity and/or function of engineered cells as described herein, when administered to a subject in adoptive therapy methods. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time (e.g., no more than 3 days). Thus, in some contexts, the first or consecutive dose is a single or continuous administration of the specified number of cells, given or initiated at a single point in time. In some contexts, however, the first or consecutive dose is administered in multiple injections or infusions over a limited time period (e.g., no more than three days), such as once a day for three days or for two days or by multiple infusions over a single day period.

The cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses can be administered in a plurality of compositions over a period of no more than 3 days.

With reference to a prior dose, such as a first dose, the term “consecutive dose” refers to a dose that is administered to the same subject after the prior, e.g., first, dose without any intervening doses having been administered to the subject in the interim. Nonetheless, the term does not encompass the second, third, and/or so forth, injection or infusion in a series of infusions or injections comprised within a single split dose. Thus, unless otherwise specified, a second infusion within a one, two or three-day period is not considered to be a “consecutive” dose as used herein. Likewise, a second, third, and so-forth in the series of multiple doses within a split dose also is not considered to be an “intervening” dose in the context of the meaning of “consecutive” dose. Thus, unless otherwise specified, a dose administered a certain period of time, greater than three days, after the initiation of a first or prior dose, is considered to be a “consecutive” dose even if the subject receives a second or subsequent injection or infusion of the cells following the initiation of the first dose, so long as the second or subsequent injection or infusion occurred within the three-day period following the initiation of the first or prior dose.

Thus, unless otherwise specified, multiple administrations of the same cells over a period of up to 3 days is considered to be a single dose, and administration of cells within 3 days of an initial administration is not considered a consecutive dose and is not considered to be an intervening dose for purposes of determining whether a second dose is “consecutive” to the first.

In some embodiments, multiple consecutive doses are given, in some aspects using the same timing guidelines as those with respect to the timing between the first dose and first consecutive dose, e.g., by administering a first and multiple consecutive doses.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×10⁶ cells/kg body weight of the subject and 5×10⁶ cells/kg, between about 0.75×10⁶ cells/kg and 3×10⁶ cells/kg or between about 1 ×10⁶ cells/kg and 2×10⁶ cells/kg.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, multiple doses can be administered to a subject over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer).

### EXAMPLES

The invention is further described in the following examples, which do not limit the scope of the invention described in the claims.

### Example 1

### Construct Design

MP71 retroviral vectors were constructed to encode the A02-A09 CARs including a single-chain variable fragment (scFv), a hinge region, a transmembrane region, a costimulatory signaling region and an intracellular signaling region. FIGS. 1A-1C show the plasmid map of retroviral vectors encoding the A02, A03 and A06 CAR, respectively. In each CAR, the scFv includes a heavy chain variable region (VH) that is linked to a light chain variable region (VL) by a linker peptide (SEQ ID NO: 37). Both the hinge region and the transmembrane region are from human CD8. The costimulatory signaling region is from human CD137 (4-1BB) and the intracellular T cell signaling domain is from CD247 (CD3 zeta)'s cytoplasmic domain.

FIG. 1D shows the plasmid map of a retroviral vector of the A02P03 vector, which further encodes an anti-PD-1 scFv that is linked to the A02 CAR by a T2A sequence (SEQ ID NO: 41). More specifically, the anti-PD-1 scFv comprises a human IL-2 leader sequence (SEQ ID NO: 42), followed by a heavy chain variable region (VH; SEQ ID NO: 15) that is linked to a light chain variable region (VL; SEQ ID NO: 16) by a linker peptide (SEQ ID NO: 87).

Similarly, FIG. lE shows the plasmid map of a retroviral vector of the A02PL01 vector, which further encodes an anti-PD-L1 scFv that is linked to the A02 CAR by a T2A sequence (SEQ ID NO: 41). More specifically, the anti-PD-L1 scFv comprises a human IL-2 leader sequence (SEQ ID NO: 42), followed by a light chain variable region (VL; SEQ ID NO: 32) that is linked to a heavy chain variable region (VH; SEQ ID NO: 31) by a linker peptide (SEQ ID NO: 87).

The retroviral vectors described above further comprises 5′LTR (5′ long terminal repeats), a Ψ RNA packaging signal (or retroviral psi packaging element), a Woodchuck hepatitis virus posttranscriptional regulatory element (WPRE), and 3′LTR (long terminal repeats). WPRE is a DNA sequence that, when transcribed creates a tertiary structure enhancing expression. The retroviral vectors also comprise a selectable marker gene, i.e., an ampicillin resistance gene. The ALPP scFv sequences were synthesized in IDT as gBlocks™ with flanking Notl and SnaBI restriction sites and subcloned to the retrovirus vector MP71 at the 5′ of CD8 hinge-coding region.

### Example 2

### Retroviral Vector Production, T Cell Transduction and Expansion

HEK-293T and SiHa cells were purchased from ATCC. Peripheral blood mononuclear cells (PBMCs) from anonymous donors were purchased from StemCell Technologies Inc. HEK-293T, Jurkat, SiHa, or peripheral blood mononuclear cells (PBMCs) were cultured in Dulbecco's Modified Eagle Medium (DMEM) supplemented with 10% fetal bovine serum (FBS), Roswell Park Memorial Institute (RPMI) medium supplemented with 10% FBS, or X-Vivo (Lonza, Cat #: 04-418Q) supplemented with 5% human serum A/B. Some cultures also included 1% HEPES and 1% GlutaMAX™.

Retroviral vectors were prepared by transient transfection of 293T cells using a standard calcium phosphate precipitation protocol. Specifically, CAR retroviruses were generated by transfecting 293T cells with ALPP CAR retrovirus construct, Gagpol and RD114 at the ratio of 4:2:1.25 with lipofectamine 2000 (Thermo Fisher Scientific, Cat#:11668500). Four hours after transfection, fresh medium was changed. 48 hours later, the medium containing viruses was collected and filtered with a 0.44 um filter.

Before retroviral transduction, PBMCs were activated with anti-human CD3/CD28 Dynabeads™ (Thermo Fisher Scientific, Cat#: 11141D) at a 3:1 ratio (beads: T cells) for 2 days. For transduction, freshly harvested retroviral supernatant was spin-loaded onto non-tissue culture-treated 24-well plates coated with 15-25 μg RetroNectin per/well (Clontech Laboratories) by centrifugation for 2 hours at 2,000 g at 32° C. Activated PBMCs were loaded onto the plates and spun at 600 g at 32° C. for 20-30 minutes.

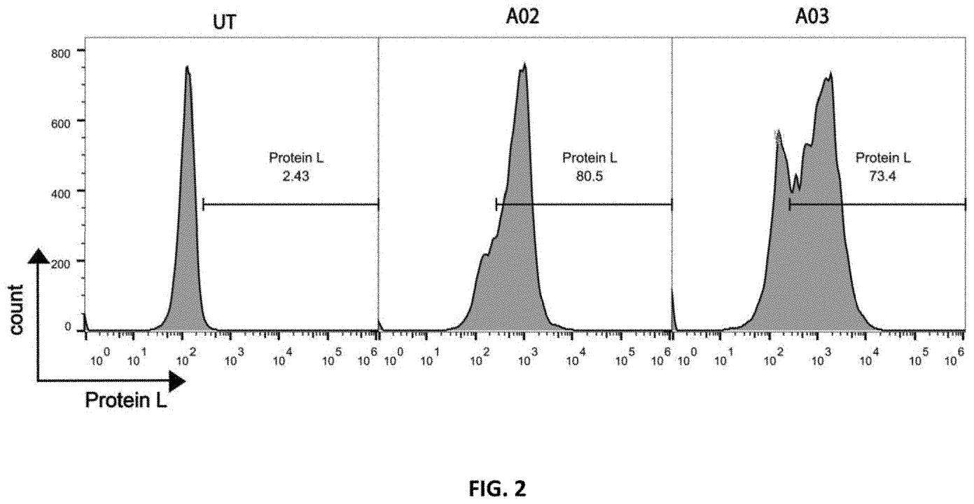

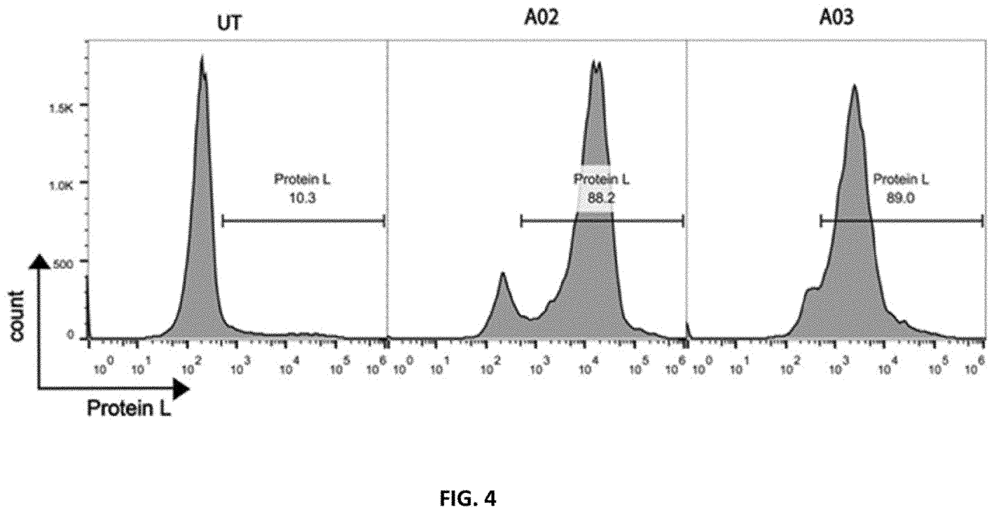

T cells were incubated at 37° C. and 5% CO₂. Culture medium was replenished every 2 days. Expression of the recombinant CARs were detected 4 days after the transduction by protein L staining followed by flow cytometry analysis (FIG. 2 and FIG. 4). CAR expression in Jurkat cells (FIG. 2) or human PBMCs (FIG. 4) were determined in a range between 70-90%.

### Example 3

### CAR-T Cell Activation

0.2×10⁶ Jurkat cells expressing A02 or A03 CAR were co-cultured with 0.4×10⁶ SiHa or 293T cells overnight, after which CD69 expression was measured by flow cytometry in CD3⁺ Jurkat cells. As shown in FIG. 3, about 70-80% Jurkat cells expressing the A02 or A03 CAR were activated when co-cultured with SiHa cells. In contrast, the A02 or A03 CAR-T cells were not activated when co-cultured with 293T cells.

### Example 4

### In Vitro IFN-γ Production and Competitive Killing Assay

0.2×10⁶ untransduced, human A02 or A03 CAR-T cells were co-cultured overnight with 0.4×10⁶ SiHa or HEK293T target cells at a 1:2 effector-to-target ratio. T cells were treated with brefeldin A (Thermo Fisher Scientific, Cat#: 00-4506-51) and monensin (Thermo Fisher Scientific, Cat#: 00-4505-51) for 4 hours, after which levels of intracellular IFN-γ were measured by flow cytometry. Briefly, the cells were collected and cell surface staining was performed by adding anti-human CD3, anti-human CD4 and anti-human CD8 antibodies mixture and incubating at room temperature for 15 minutes. After surface staining, the cells were fixed with Cytofix™ solution (BD, Cat#: 554714) at room temperature for 15 minutes, washed once with PBS, and permeabilized with 0.1 ml 1× Perm/Wash buffer (BD, Cat#: 554714) for 10 minutes at room temperature. After washing once with 1 ml Perm/Wash buffer, the cells were stained with an anti-IFN-γ antibody in 80 μL of Perm/Wash buffer at room temperature for 30 min, washed once with 1 ml of Perm/Wash buffer and resuspended with PBS for flow cytometry. A viable CD4⁺ or CD8⁺ lymphocyte gating strategy was used (FIG. 5 and FIG. 6). The intracellular IFN-γ expression results indicated that CAR-T cells containing the A02 or A03 CAR were specifically activated by ALPP-positive SiHa cells.

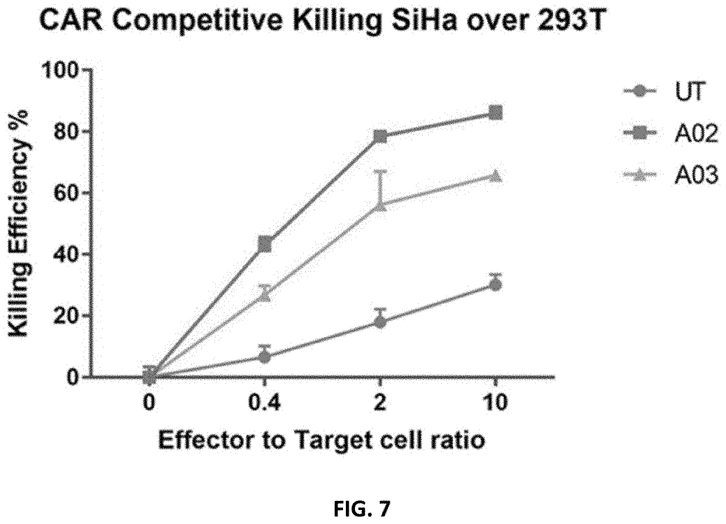

Furthermore, cytotoxicity assay was performed in a U-shape 96-well plate with four replicates for each condition. For each well, 0.03×10⁶ SiHa cells labeled with CellTrace™ CFSE (Thermo Fisher Scientific, Cat#: C34554) and 0.03×10⁶ HEK293T cells labeled with CellTrace™ Violet (Thermo Fisher Scientific, Cat#: C34557) were mixed and co-cultured overnight with untransduced, A02, or A03 CAR-T cells at increasing effector-to-target cell ratios. Live SiHa and 293T cells were quantified by flow cytometry testing pre-labeled dyes, and competitive killing efficiency was calculated based on the live SiHa/293T cell ratio (FIG. 7). Further, the results indicate that A02 and A03 CAR-T cells specifically killed SiHa cells. Hence, the above findings suggest that anti-ALPP CAR-T cell therapy is an effective therapeutic approach for the treatment of ALPP-positive tumors.

### Example 5

### In Vivo Tumor Implantation and Treatment

6-8-week-old female NSG mice were intraperitoneally implanted with 5.0×10⁶ SiHa cells. 34 days later (study day 0), animals were grouped based on body weight and the presence of clinical signs indicating tumor growth. On study day 0, all animals were intraperitoneally injected with 10⁶ CAR^{P} A02 cells, A03 cells, or an equivalent number of untransduced cells (11.78×10⁶ cells/mouse). Moreover, overall survival was assessed based on animals hitting study endpoint. Study endpoint was defined as death, moribundity, a severe decrease in body condition with a body conditioning score less than 2, severe abdominal distension that interfered with animals' ability to ambulate normally, or a body weight gain more than 20%. As shown in FIG. 8, the results indicate that no toxicity was associated with the CAR-T treatment. Results in FIG. 9 indicates that treatment with either A02 or A03 CAR-T cells significantly improved survival rate.

### Example 6

### Humanization of Anti-ALPP Antibodies

In general, antibody specific sequences (CDRs) from the mouse parent antibody H17E2 (or A02) were grafted onto human donor sequences. The donor sequences were selected according to bioinformatics software that utilizes extensive antibody database to calculate a unique humanness score for each combination. These antibodies were cloned into an expression vector and transfected into cells lines for recombinant protein expression. Antibody affinity to the target protein, ALPP, was tested in vitro by ELISA and cell-based binding assays.

The humanization of antibodies was achieved by CDR grafting and resurfacing strategies. Moreover, de-immunization strategy was also used. Bioinformatics tools including antibody modelling and critical framework residues identification were utilized to design humanized heavy and light chains. Those with the maximal humanization scores were combined to obtain humanized antibodies A03, A04, A05, A06, A07, A08, and A09.

The humanized VH and VL were cloned into transient expression vectors. The final constructs were confirmed by sequencing. Further, plasmids encoding the candidate pairs (including one chimeric pair for control) of heavy and light chain were co-transfected into CHO or HEK293 cells for transient expression. The expressed antibodies were purified, followed by measurement of their epitope specificity and affinity by ELISA or cell based-binding assay.

### Example 7

### Binding Affinities of Humanized Anti-ALPP Antibodies

Binding affinities of anti-ALPP antibodies including the parent mouse antibody A02, and humanized antibodies A04, A05, A06, A07, A08, and A09, were determined by ELISA or cell-based binding assays. A chimeric antibody was used as a control. Detailed methods are provided as follows.

### ELISA

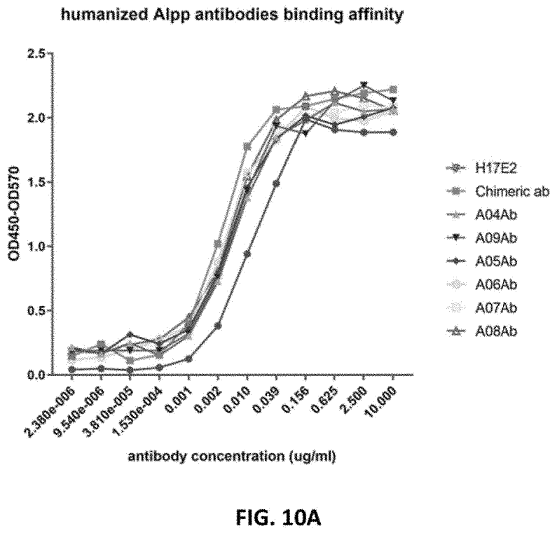

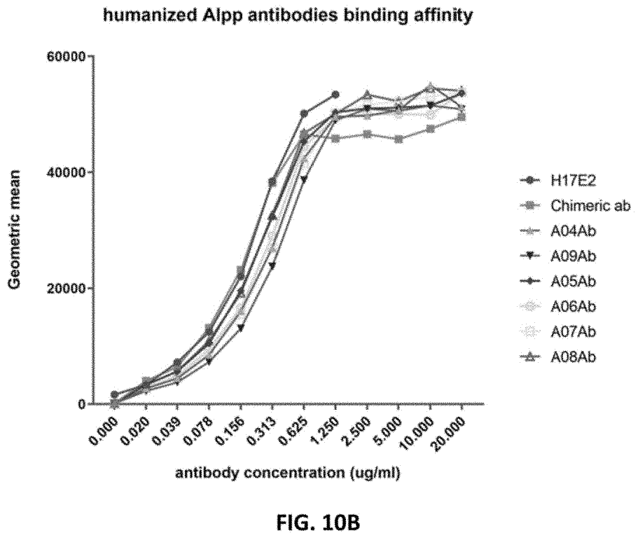

Enzyme-linked immunosorbent assay (ELISA) plates were coated with 125 ng ALPP antigen. After blocking the plates, serial dilutions of anti-ALPP antibodies including A02 (H17E2), A04, A05, A06, A07, A08, A09, or a chimeric antibody were added to corresponding wells. Then, a Biotin-SP-AffiniPure goat anti-human antibody was added to wells containing the humanized antibodies A04, A05, A06, A07, A08, A09, or the chimeric antibody. Meanwhile, a Biotin-SP-AffiniPure goat anti-mouse Fc antibody was added to wells containing the mouse antibody H17E2. Next, HRP-conjugated streptavidin was used for biotin detection. Final detection was accomplished by assessing OD450 after 3,3′,5,5′-Tetramethylbenzidine (TMB) substrate was added. Binding curves and calculated EC₅₀ values are shown in FIG. 10A and the table below, respectively. Comparable EC₅₀ values were obtained for the chimeric and humanized anti-ALPP antibodies with respect to the parent mouse anti-ALPP antibody H17E2.

TABLE 1

| Antibody | H17E2 | Chimeric Ab | A04Ab | A09Ab | A05Ab | A06Ab | A07Ab | A08Ab |
|---|---|---|---|---|---|---|---|---|
| EC₅₀ (μg/ml) | 0.01079 | 0.00308 | 0.00589 | 0.00564 | 0.00491 | 0.00370 | 0.00534 | 0.00481 |

### Cell-Based Binding Assays

0.2×10⁶ SiHa cells were incubated with serially diluted anti-ALPP antibodies as described herein. Afterward, an AF488 goat anti-human secondary antibody or an AF488 goat anti-mouse IgG (Fcγ fragment specific) was used to stain the cells. Then, the fluorescently stained cells were detected by flow cytometry. Binding curves and calculated EC₅₀ values are shown in FIG. 10B and the table below, respectively. Comparable EC₅₀ values were obtained by cell-based binding assays for the chimeric and humanized anti-ALPP antibodies with respect to the parent mouse anti-ALPP antibody H17E2.

TABLE 2

| Antibody | H17E2 | Chimeric Ab | A04Ab | A09Ab | A05Ab | A06Ab | A07Ab | A08Ab |
|---|---|---|---|---|---|---|---|---|
| EC₅₀ (μg/ml) | 0.2113 | 0.1542 | 0.2864 | 0.3382 | 0.2253 | 0.2572 | 0.3022 | 0.2334 |

### Example 8

### Expression and Activity of Humanized Anti-ALPP CARs in T Cells

### CAR Expression Levels in Human T Cells

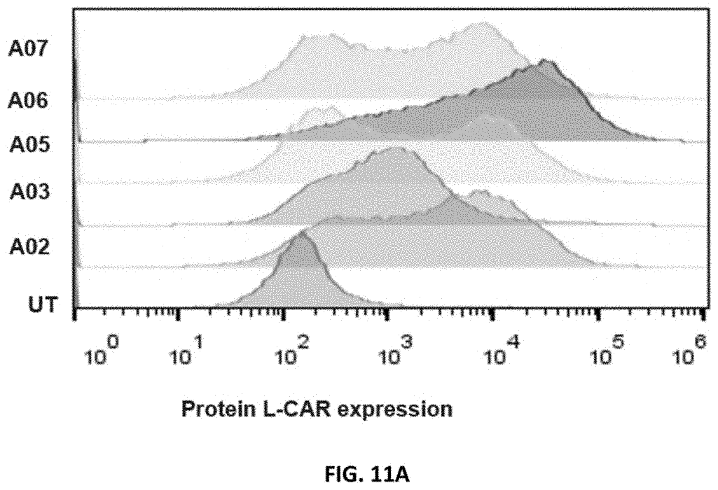

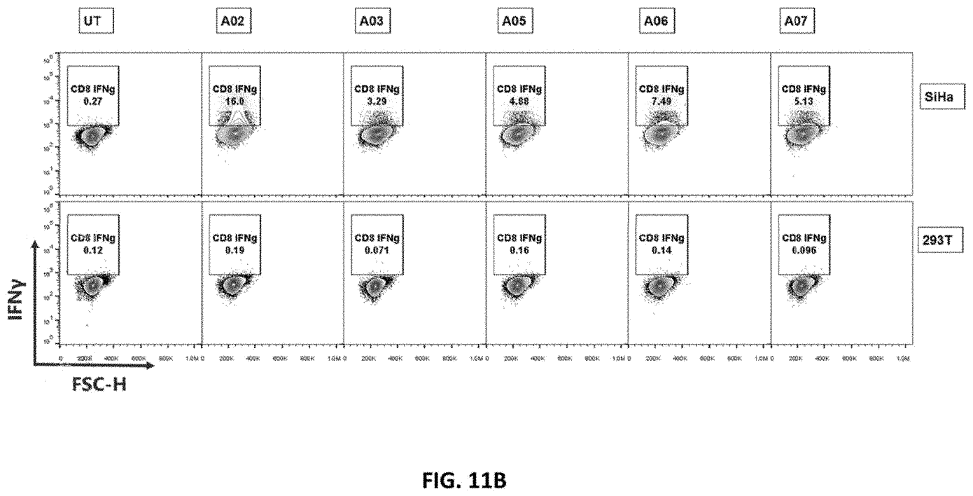

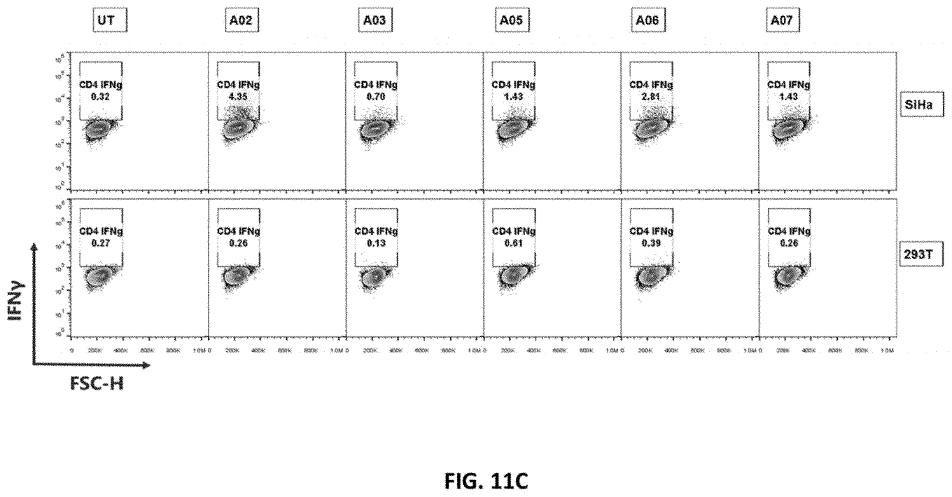

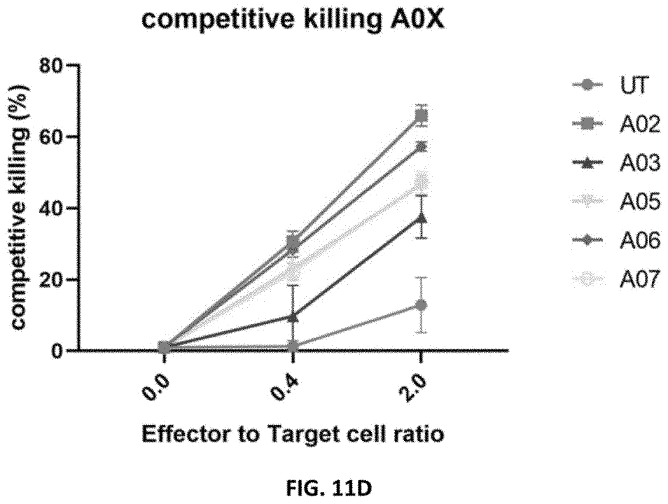

Human PBMCs were transduced with retroviral plasmids to express A02, A03, A05, A06, or A07 CARs, which were constructed from the corresponding humanized antibodies. 12 days post-transduction, CAR expression levels were measured by protein L assays. As shown in FIG. 11A, all the humanized CARs were expressed in the transduced PBMCs.

### IFN-γ Expression Assay

0.2×10⁶ untransduced (UT), A02, A03, A05, A06 or A07 CAR-T cells were co-cultured overnight with 0.4×10⁶ SiHa or 293T cells. Cells were then treated with Brefeldin A and Monensin for 4 hours, after which intracellular IFNγ levels were measured by flow cytometry. Both CD8⁺ and CD4⁺ T cell populations were analyzed, as shown in FIG. 11B and FIG. 11C, respectively.

### Competitive Killing Assay

0.03×10⁶ SiHa cells were labeled with CellTrace™ CFSE and 0.03×10⁶ 293T cells were labeled with CellTrace™ Violet. Labeled SiHa cells and 293T cells were mixed and co-cultured overnight with untransduced (UT), A02, A03, A05, A06, or A07 CAR-T cells at increasing effector-to-target cell ratios, as shown in FIG. 11D. The experiment was carried out with 4 replicates in 96-well plates. Live SiHa and 293T cells were quantified by flow cytometry, and competitive killing efficacy was calculated based on numbers of live SiHa and 293T cells

### Example 9

### Expression and Activity of Humanized Anti-ALPP CARs in Combination with Immune Checkpoint Inhibition

### CAR Expression Levels in Human T Cells

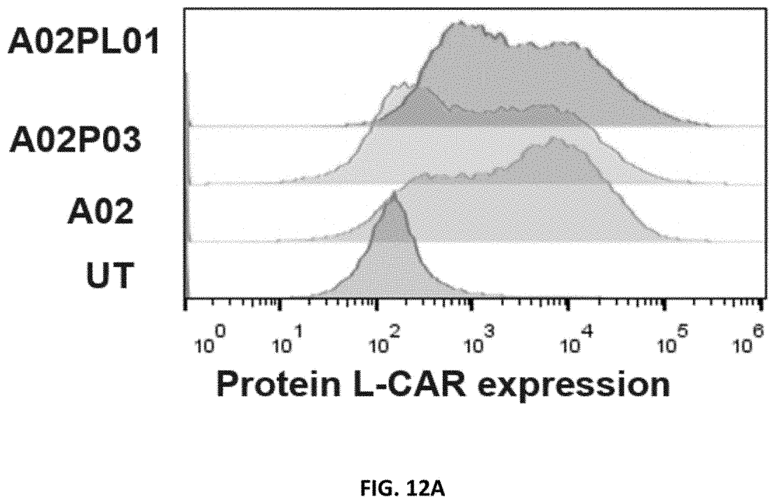

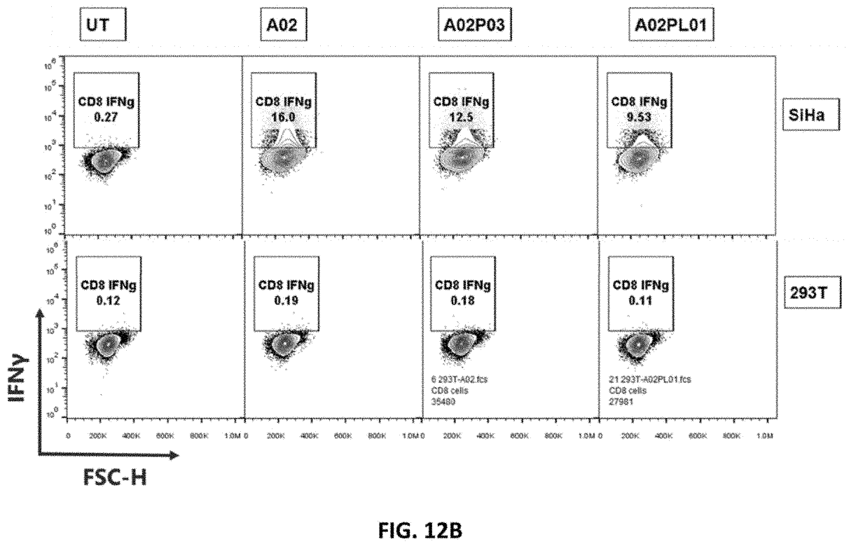

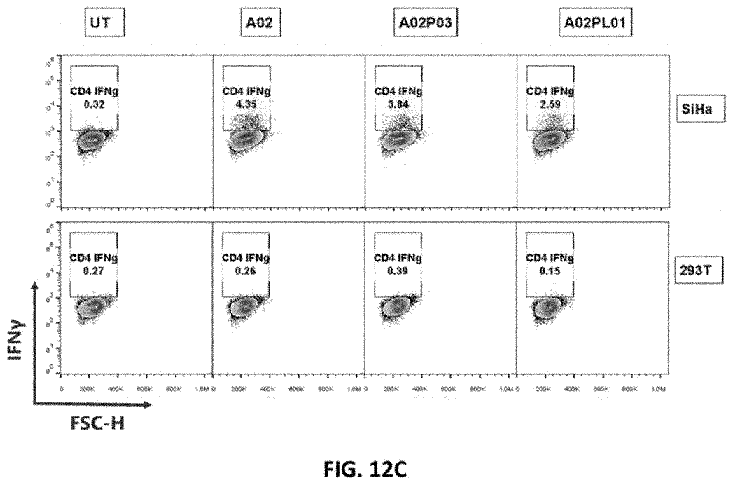

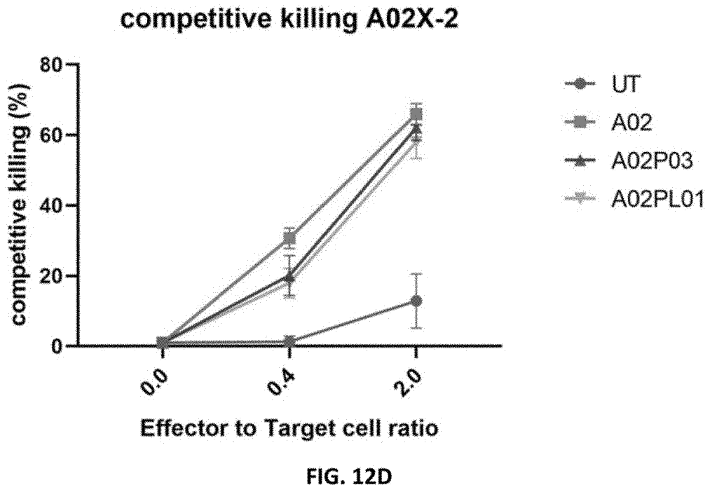

Human PBMCs were transduced with A02, A02P03, or A02PL01 retrovirus. 12 days post transduction, CAR expression levels were measured by protein L assay. As shown in FIG. 12A, CAR expression was detected in the transduced PBMCs.

### IFN-γ Expression Assay

0.2×10⁶ untransduced (UT), A02, A02P03, or A02PLO1 CAR-T cells were co-cultured overnight with 0.4×10⁶ SiHa or 293T cells. Cells were then treated with Brefeldin A and Monensin for 4 hours, after which intracellular IFN-γ levels were measured by flow cytometry. Both CD8⁺ and CD4⁺ T cell populations were analyzed, as shown in FIG. 12B and FIG. 12C, respectively.

### Competitive Killing Assay

0.03×10⁶ SiHa cells were labeled with CellTrace™ CFSE and 0.03×10⁶ 293T cells were labeled with CellTrace™ Violet. Labeled SiHa cells and 293T cells were mixed and co-cultured overnight with untransduced (UT), A02, A02P03, or A02PL01 CART cells at increasing effector-to-target cell ratios, as shown in FIG. 12D. The experiment was carried out with 4 replicates in 96-well plates. Live SiHa and 293T cells were quantified by flow cytometry, and competitive killing efficacy was calculated based on numbers of live SiHa and 293T cells.

### Example 10

### In Vivo Efficacy and Toxicity of Anti-ALPP CAR-T Cells

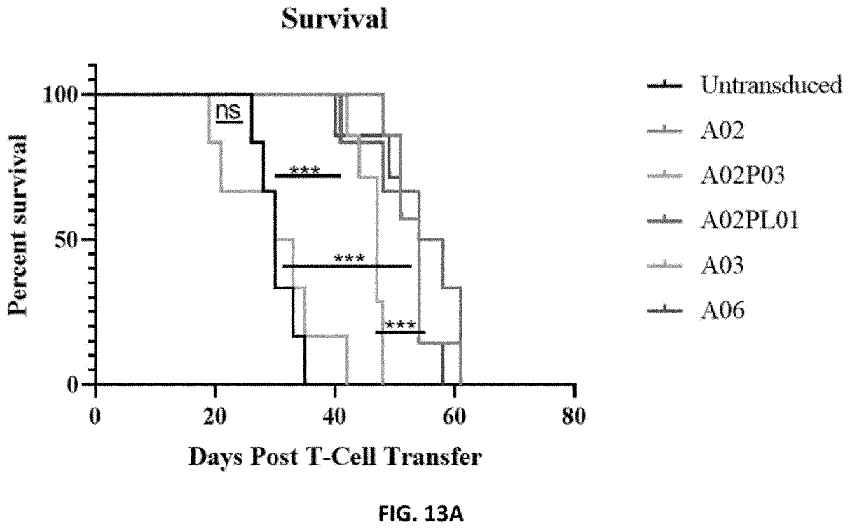

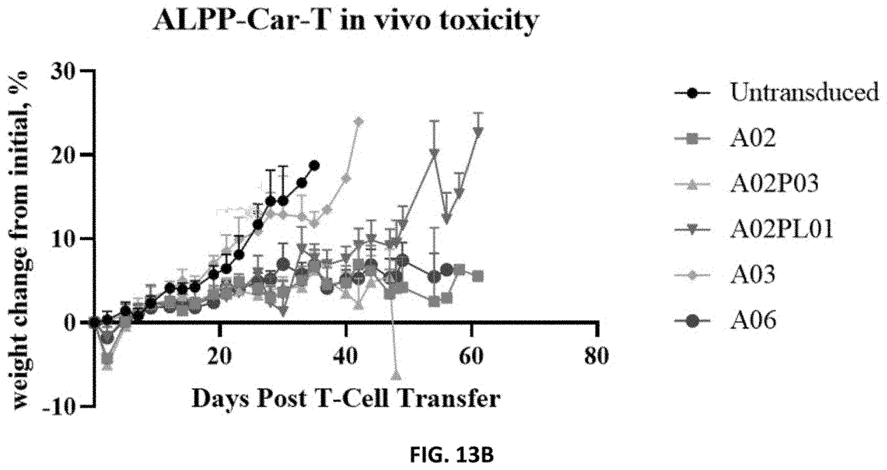

A total of 39 female, 10-week-old NSG mice were intraperitoneally implanted with 5.0×10⁶ SiHa cells in 200 μl PBS. 40 days later (on study Day 0), the animals were sorted into groups based on body weights and the presence of clinical signs indicating tumor growth, and then intraperitoneally injected with 10⁶ CAR-positive A02, A02P03, A02PL01, A03, A06, or the equivalent number of untransduced cells (17.08×10⁶ cells/mouse). Overall survival was plotted based on when animals hit study endpoints, as determined by death, moribundity, a severe decrease in body condition with a body conditioning score less than 2, severe abdominal distension that interfered with animals' ability to ambulate normally, or a body weight gain more than 20%. Antitumor efficacy and toxicity as measured by animal survival and mouse body weights are shown in FIG. 13A and FIG. 13B, respectively. Details of determining body conditioning scores can be found, e.g., in Ullman-Culleré et al., “Body condition scoring: a rapid and accurate method for assessing health status in mice.” Comparative Medicine 49.3 (1999): 319-323; Charmaine et al., “Guidelines for assessing the health and condition of mice.” Lab Animal 28.5 (1999); both of which are incorporated herein by reference in its entirety.

### Example 11

### Modification of A02 CAR

### Design of Modified A02 CAR

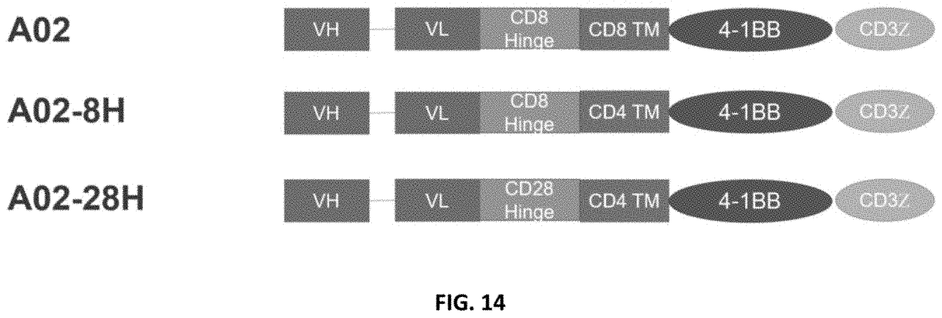

As shown in FIG. 14, the sequence encoding the A02 CAR (VH: SEQ ID NO: 1; VL: SEQ ID NO: 98) was modified to express A02-8H and A02-28H CARs. Specifically, the A02-8H CAR includes a heavy chain variable region (VH; SEQ ID NO: 1) that is linked to a light chain variable region (VL; SEQ ID NO: 98) by a linker peptide (SEQ ID NO: 37), followed by a CD8 hinge region (SEQ ID NO: 94), a CD4 transmembrane region (SEQ ID NO: 96), a costimulatory signaling region from human 4-1BB (SEQ ID NO: 39), and an intracellular T cell signaling domain from CD3 zeta's cytoplasmic domain (SEQ ID NO: 40).

The A02-28H CAR includes a heavy chain variable region (VH; SEQ ID NO: 1) that is linked to a light chain variable region (VL; SEQ ID NO: 98) by a linker peptide (SEQ ID NO: 37), followed by a CD28 hinge region (SEQ ID NO: 97), a CD4 transmembrane region (SEQ ID NO: 96), a costimulatory signaling region from human 4-1BB (SEQ ID NO: 39), and an intracellular T cell signaling domain from CD3 zeta's cytoplasmic domain (SEQ ID NO: 40).

The A02, A02-8H and A02-28H constructs were cloned into a MP71 retroviral vector to produce CAR-T cells using the methods described herein.

### In Vitro Expression of ALPP CAR

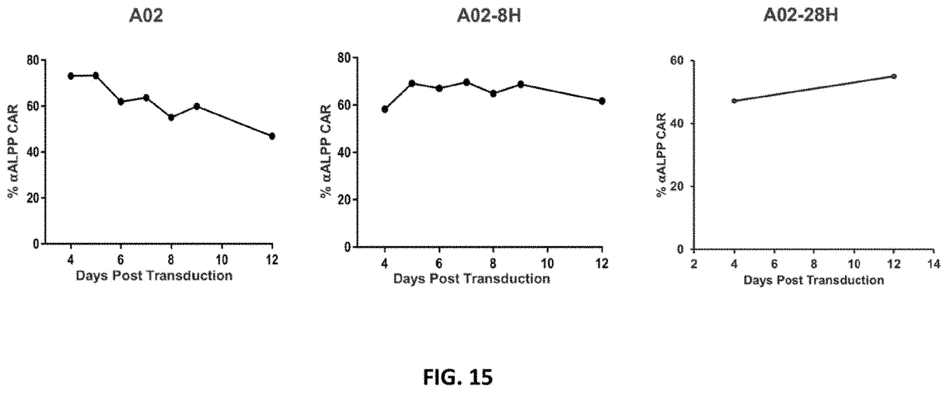

As shown in FIG. 15, primary human T cells were transduced with the A02, A02-8H, or A02-28H constructs. After transduction, CAR expression was measured at indicated time points by ALPP-His protein staining. Specifically, 0.2×10⁶ untransduced or transduced cells were collected for CAR expression level test. The cells were stained with 0.1 μg ALPP protein (Novus Biologicals, Cat#: NBP2-52266) for 15 minutes at room temperature, washed once with PBS and stained with an anti-His antibody for 15 minutes at room temperature. The stained cells were washed once with PBS, resuspended with PBS and subjected to flow cytometry analysis. A viable lymphocyte gating strategy was used. The results indicate that modified A02 CARs (A02-8H and A02-28H) could be more stably expressed on the T cell surface than the A02 CAR.

### ALPP CAR-T Cell Basal Activation Without Antigen Stimulation

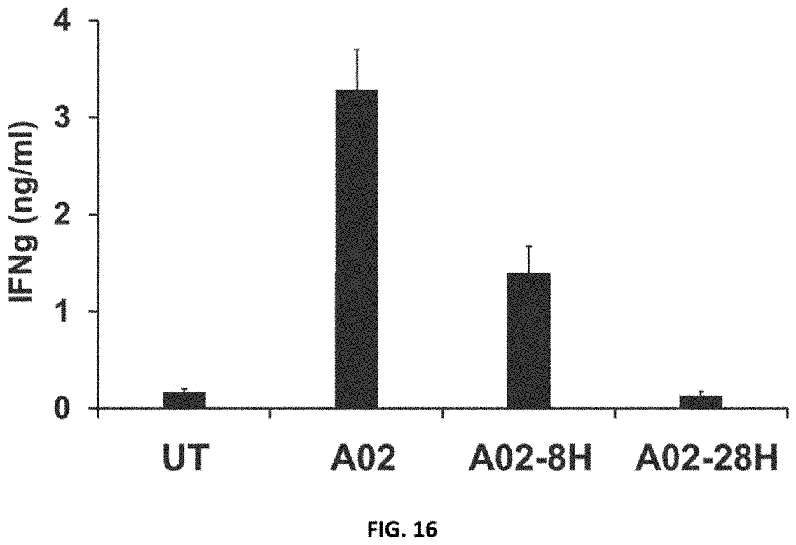

Primary human T cells were untransduced or transduced with the A02, A02-8H, or A02-28H constructs. 12 days post transduction, the CAR-T cells were seeded in a 96-well plate at 0.03×10⁶/well in 200 μl of T cell culture medium. After 48 hours of culturing, the supernatant was harvested for determination of IFN-γ level by ELISA. As shown in FIG. 16, both A02-8H and A02-28H CAR-T cells showed a lower basal activation level as compared to A02 CAR-T cells. The lower basal T cell activation can reduce undesired side effects of CAR-T therapy and potentially improve the overall antitumor efficacy.

### Proliferation of ALPP CAR-T Cell

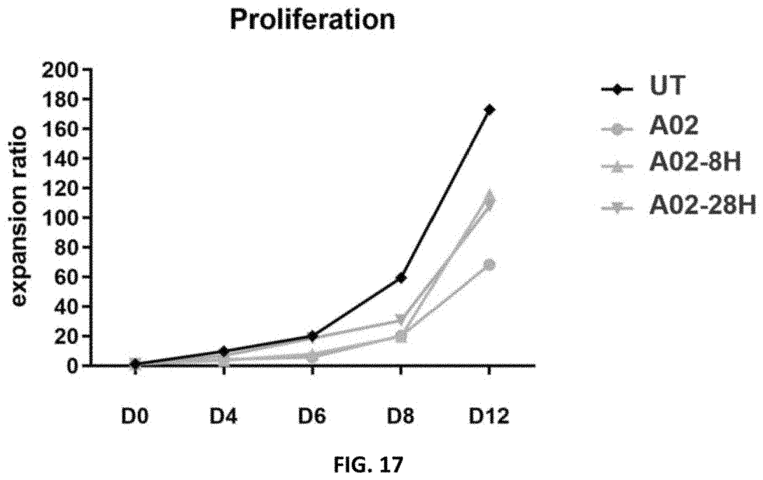

Primary human T cells were transduced with the A02, A02-8H, or A02-28H constructs. The CAR-T cell number was counted on day 0, day 4, day 6, day 8, and day 12 post transduction. As shown in FIG. 17, both A02-8H and A02-28H CAR-T cells showed an increased proliferation as compared to that of the A02 CAR-T cells.

### ALPP CAR-T Cell Activation Upon Antigen-Specific Stimulation

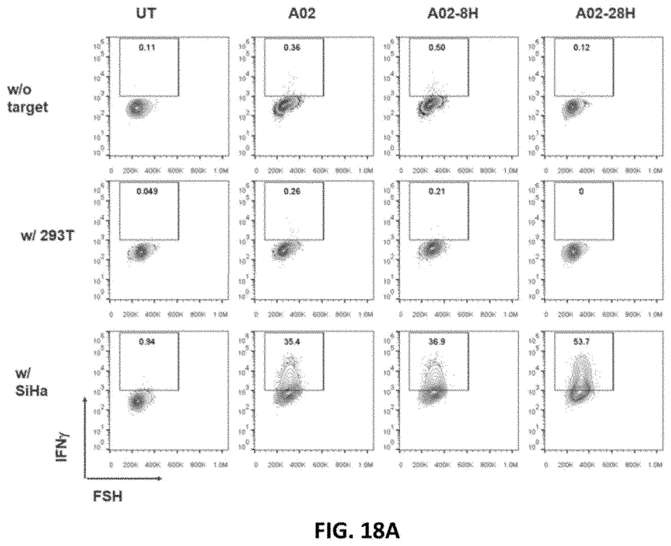

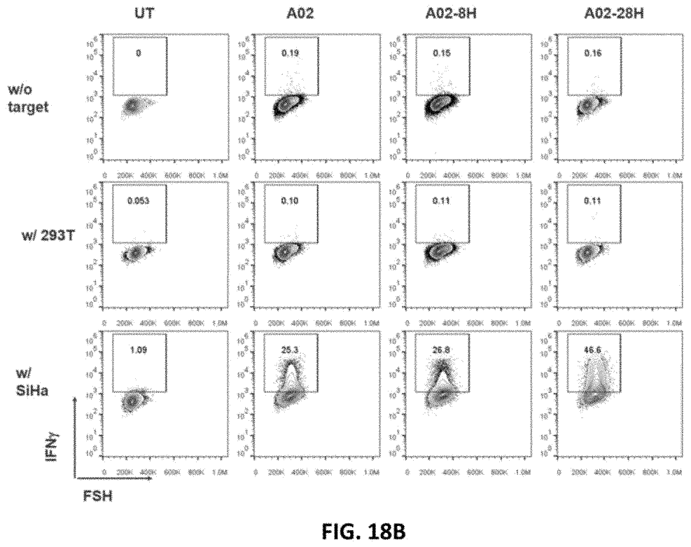

Primary human T cells were untransduced or transduced with the A02, A02-8H, or A02-28H constructs. The CAR-T cells were cultured without tumor cells (Blank), with non-target tumor cells (293T), or with target tumor cells (SiHa) overnight. Afterwards, the T cells were collected and intracellular IFN-γ expression was measured using the methods described herein. Either CD3⁺CD8⁺ (FIG. 18A) or CD3⁺CD4⁺ (FIG. 18B) cells were analyzed. The results showed that both A02-8H and A02-28H CAR-T cells, especially A02-28H CAR-T cells, could be better activated by the target tumor cells than the A02 CAR-T cells.

### ALPP CAR-T Cell Specific Cytotoxicity Against Target Tumor Cells

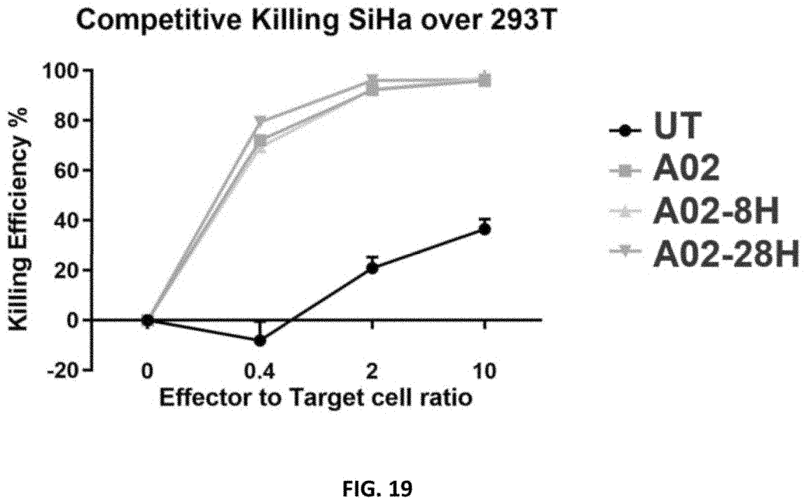

Primary human T cells were untransduced or transduced with the A02, A02-8H, or A02-28H constructs. The CAR-T cells were cultured with CFSE pre-labeled non-target tumor cells (293T) or target tumor cells (SiHa) overnight. Afterwards, the tumor cells were collected, and their viability was measured using 7-Aminoactinomycin D (7-AAD). As shown in FIG. 19, the competitive killing efficacy of both A02-8H and A02-28H CAR-T cells was similar to that of the A02 CAR-T cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating cancer, comprising:
administering an effective amount of genetically engineered anti-tumor human T cells to a patient to treat cancer of the patient, wherein the anti-tumor human T cells have been obtained by incorporating a recombinant DNA sequence encoding a CAR into T cells extracted from the patient, wherein the encoded CAR comprises an ALPP antigen binding domain that binds to ALPP expressed in cancer cells.

2. The method of treating a patient of claim 1, wherein the cancer is lung cancer.

3. The method of treating a patient of claim 1, wherein the cancer is gastric cancer.

4. The method of treating a patient of claim 1, wherein the cancer is pancreatic cancer.

5. The method of treating a patient of claim 1, wherein the cancer is head & neck cancer.

6. The method of treating a patient of claim 1, wherein the cancer is colorectal cancer.

7. The method of treating a patient of claim 1, wherein the cancer is urothelial cancer.

8. The method of treating a patient of claim 1, wherein the cancer is renal cancer.

9. The method of treating a patient of claim 1, wherein the cancer is cancer of reproductive organs.

10. The method of treating a patient of claim 8, wherein the cancer of reproductive organs is ovarian cancer.

11. The method of treating a patient of claim 8, wherein the cancer of reproductive organs is endometrial cancer.

12. The method of treating a patient of claim 8, wherein the cancer of reproductive organs is cervical cancer.

13. The method of treating a patient of claim 8, wherein the cancer of reproductive organs is testicular cancer.

14. A method of treating cancer, comprising:
administering an effective amount of genetically engineered anti-tumor human T cells to a patient to treat cancer of the patient, wherein the anti-tumor human T cells have been obtained by incorporating a recombinant DNA sequence encoding a CAR into T cells extracted from the patient, wherein the encoded CAR comprises an ALPP antigen binding domain;
wherein the CAR-T cell antigen binding domain consists of an antibody or antibody fragment;
wherein the said antibody has a variable heavy chain region selected from SEQ ID NO: 1 or SEQ ID NO: 3; and a variable light chain region selected from SEQ ID NO: 2 or SEQ ID NO: 4.

15. A method of treating cancer of claim 14, wherein the antibody is murine antibody against ALPP having a variable heavy chain region SEQ ID NO: 1 and variable light chain region SEQ ID NO: 2 or 98.

16. A method of treating cancer of claim 14, wherein the antibody is humanized antibody against ALPP having a variable heavy chain region SEQ ID NO: 3 and variable light chain region SEQ ID NO: 4.

17. A chimeric antigen receptor comprising: (a) an extracellular antigen-binding domain that specifically recognizes alkaline phosphatase, placental (ALPP); (b) a transmembrane domain;
and (c) an intracellular signaling region.

18. The chimeric antigen receptor of claim 17, wherein the antigen-binding domain comprises a heavy chain variable domain (VH) and a light chain variable domain (VL).

19. The chimeric antigen receptor of claim 18, wherein the VH comprises heavy chain complementarity determining regions (CDRs) 1, 2, and 3 and the VL comprises VL CDRs 1, 2, and 3,
wherein the VH CDRs 1, 2, and 3 amino acid sequences and the VL CDRs, 1, 2, and 3 amino acid sequences are one of the following:
(1) the VH CDRs 1, 2, and 3 amino acid sequences are set forth in SEQ ID NOs: 45, 46, and 47, respectively, and the VL CDRs 1, 2, and 3 amino acid sequences are set forth in SEQ ID NOs: 48, 49, and 50, respectively;
(2) the VH CDRs 1, 2, and 3 amino acid sequences are set forth in SEQ ID NOs: 51, 52, and 53, respectively, and the VL CDRs 1, 2, and 3 amino acid sequences are set forth in SEQ ID NOs: 54, 55, and 56, respectively;
(3) the VH CDRs 1, 2, and 3 amino acid sequences are set forth in SEQ ID NOs: 57, 58, and 59, respectively, and the VL CDRs 1, 2, and 3 amino acid sequences are set forth in SEQ ID NOs: 60, 61, and 62, respectively;
(4) the VH CDRs 1, 2, and 3 amino acid sequences are set forth in SEQ ID NOs: 63, 64, and 65, respectively, and the VL CDRs 1, 2, and 3 amino acid sequences are set forth in SEQ ID NOs: 66, 67, and 68, respectively; and
(5) the VH CDRs 1, 2, and 3 amino acid sequences are set forth in SEQ ID NOs: 81, 82, and 83, respectively, and the VL CDRs 1, 2, and 3 amino acid sequences are set forth in SEQ ID NOs: 84, 85, and 86, respectively.

20. The chimeric antigen receptor of claim 18 or 19, wherein the VH consist of or comprises an amino acid sequence having at least 90%, 91%, 92%, 93%, 94%, 95%, 96%, 97%, 98%, 99% or 100% sequence identity to SEQ ID NOs: 1, 3, 5, 7, 9, 11, or 13; and the VL consists of or comprises an amino acid sequence having at least 90%, 91%, 92%, 93%, 94%, 95%, 96%, 97%, 98%, 99% or 100% sequence identity to SEQ ID NOs: 2, 4, 6, 8, 10, 12, 14, or 98.

21. The chimeric antigen receptor of claim 20, wherein the VH comprises an amino acid sequence having at least 90% identity to SEQ ID NO: 1 and the VL comprises an amino acid sequence having at least 90% identity to SEQ ID NO: 2 or 98.

22. The chimeric antigen receptor of claim 20, wherein the VH comprises an amino acid sequence having at least 90% identity to SEQ ID NO: 3 and the VL comprises an amino acid sequence having at least 90% identity to SEQ ID NO: 4.

23. The chimeric antigen receptor of claim 20, wherein the VH comprises an amino acid sequence having at least 90% identity to SEQ ID NO: 5 and the VL comprises an amino acid sequence having at least 90% identity to SEQ ID NO: 6.

24. The chimeric antigen receptor of claim 20, wherein the VH comprises an amino acid sequence having at least 90% identity to SEQ ID NO: 7 and the VL comprises an amino acid sequence having at least 90% identity to SEQ ID NO: 8.

25. The chimeric antigen receptor of claim 20, wherein the VH comprises an amino acid sequence having at least 90% identity to SEQ ID NO: 9 and the VL comprises an amino acid sequence having at least 90% identity to SEQ ID NO: 10.

26. The chimeric antigen receptor of claim 20, wherein the VH comprises an amino acid sequence having at least 90% identity to SEQ ID NO: 11 and the VL comprises an amino acid sequence having at least 90% identity to SEQ ID NO: 12.

27. The chimeric antigen receptor of claim 20, wherein the VH comprises an amino acid sequence having at least 90% identity to SEQ ID NO: 13 and the VL comprises an amino acid sequence having at least 90% identity to SEQ ID NO: 14.

28. The chimeric antigen receptor of any one of claims 17-27, wherein the antigen-binding domain comprises an scFv.

29. The chimeric antigen receptor of any one of claims 17-28, wherein the VH region and the VL region are joined by a flexible linker.

30. The chimeric antigen receptor of claim 29, wherein the flexible linker comprises the amino acid sequence of EKGRSGGGGSGGGGSGGGGS (SEQ ID NO: 37).

31. The chimeric antigen receptor of claim 29, wherein the flexible linker comprises the amino acid sequence of GGGGSGGGGSGGGGS (SEQ ID NO: 87).

32. The chimeric antigen receptor of any one of claims 17-31, wherein the chimeric antigen receptor further comprises a hinge region.

33. The chimeric antigen receptor of claim 32, wherein the hinge region comprises a membrane-proximal region from IgG, CD8, or CD28.

34. The chimeric antigen receptor of claim 33, wherein the hinge region comprises a CD8 membrane-proximal region.

35. The chimeric antigen receptor of any of claims 17-34, wherein the transmembrane domain comprises a transmembrane region of CD4, CD8, or CD28.

36. The chimeric antigen receptor of claim 35, wherein the transmembrane domain comprises a CD8 transmembrane region.

37. The chimeric antigen receptor of claim 34 or 36, wherein the hinge region and/or the transmembrane region are from human CD8.

38. The chimeric antigen receptor of any one of claims 17-37, wherein the chimeric antigen receptor comprises an amino acid sequence set forth in SEQ ID NO: 38, or an amino acid sequence that is at least 90% identical to SEQ ID NO: 38.

39. The chimeric antigen receptor of any of claims 17-38, wherein the intracellular signaling region comprises an activating cytoplasmic signaling domain.

40. The chimeric antigen receptor of claim 39, wherein the activating cytoplasmic signaling domain is capable of inducing a primary activation signal in a T cell, is a T cell receptor (TCR) component, and/or comprises an immunoreceptor tyrosine-based activation motif (ITAM).

41. The chimeric antigen receptor of claims 17-40, wherein the intracellular signaling region is or comprises a functional signaling domain of CD3 zeta.

42. The chimeric antigen receptor of claim 41, wherein the CD3 zeta is human CD3 zeta.

43. The chimeric antigen receptor of claim 42, wherein the intracellular signaling region is or comprises the amino acid sequence set forth in SEQ ID NO: 40 or an amino acid sequence that is at least 90% sequence identical to SEQ ID NO: 40.

44. The chimeric antigen receptor of any of claims 17-43, wherein the intracellular signaling region further comprises a costimulatory signaling region.

45. The chimeric antigen receptor of claims 44, wherein the costimulatory signaling region is between the transmembrane domain and the intracellular signaling region.

46. The chimeric antigen receptor of claim 44 or 45, wherein the costimulatory signaling region comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/ , and CD19a.

47. The chimeric antigen receptor of claim 46, wherein the costimulatory signaling region is or comprises a functional signaling domain from OX40, CD28, 4-1BB, ICOS, or a signaling portion thereof.

48. The chimeric antigen receptor of claim 47, wherein the costimulatory signaling region comprises an intracellular signaling domain of 4-1BB.

49. The chimeric antigen receptor of claim 48, wherein the 4-1BB is human 4-1BB.

50. The chimeric antigen receptor of claim 49, wherein the costimulatory signaling region is or comprises an amino acid sequence set forth in SEQ ID NO: 39 or an amino acid sequence that is at least 90% identical to SEQ ID NO: 39.

51. The chimeric antigen receptor of claim 47, wherein the costimulatory signaling region comprises intracellular signaling domains of CD28 and 4-1BB.

52. The chimeric antigen receptor of claim 50, wherein the CD28 is human CD28 and the 4-1BB is human 4-1BB.

53. The chimeric antigen receptor of claim 52, wherein the costimulatory signaling region is or comprises an amino acid sequence set forth in SEQ ID NO: 90 or an amino acid sequence that is at least 90% identical to SEQ ID NO: 90.

54. A chimeric antigen receptor comprising an amino acid sequence having at least 90%, 91%, 92%, 93%, 94%, 95%, 96%, 97%, 98%, 99% or 100% identity to SEQ ID NO: 18, 20, 22, 24, 26, 28, 30, 91, 92, 93, 99, 101, or 103.

55. The chimeric antigen receptor of claim 54, wherein the amino acid sequence is at least 90%, 91%, 92%, 93%, 94%, 95%, 96%, 97%, 98%, 99% or 100% identical to SEQ ID NO: 18, 99, 101, or 103.

56. The chimeric antigen receptor of claim 54, wherein the amino acid sequence is at least 90%, 91%, 92%, 93%, 94%, 95%, 96%, 97%, 98%, 99% or 100% identical to SEQ ID NO: 20.

57. The chimeric antigen receptor of claim 54, wherein the amino acid sequence is at least 90%, 91%, 92%, 93%, 94%, 95%, 96%, 97%, 98%, 99% or 100% identical to SEQ ID NO: 22.

58. The chimeric antigen receptor of claim 54, wherein the amino acid sequence is at least 90%, 91%, 92%, 93%, 94%, 95%, 96%, 97%, 98%, 99% or 100% identical to SEQ ID NO: 24.

59. The chimeric antigen receptor of claim 54, wherein the amino acid sequence is at least 90%, 91%, 92%, 93%, 94%, 95%, 96%, 97%, 98%, 99% or 100% identical to SEQ ID NO: 26.

60. The chimeric antigen receptor of claim 51, wherein the amino acid sequence is at least 90%, 91%, 92%, 93%, 94%, 95%, 96%, 97%, 98%, 99% or 100% identical to SEQ ID NO: 28.

61. The chimeric antigen receptor of claim 54, wherein the amino acid sequence is at least 90%, 91%, 92%, 93%, 94%, 95%, 96%, 97%, 98%, 99% or 100% identical to SEQ ID NO: 30.

62. The chimeric antigen receptor of claim 54, wherein the amino acid sequence is at least 90%, 91%, 92%, 93%, 94%, 95%, 96%, 97%, 98%, 99% or 100% identical to SEQ ID NO: 91.

63. The chimeric antigen receptor of claim 54, wherein the amino acid sequence is at least 90%, 91%, 92%, 93%, 94%, 95%, 96%, 97%, 98%, 99% or 100% identical to SEQ ID NO: 92.

64. The chimeric antigen receptor of claim 54, wherein the amino acid sequence is at least 90%, 91%, 92%, 93%, 94%, 95%, 96%, 97%, 98%, 99% or 100% identical to SEQ ID NO: 93.

65. A polynucleotide encoding the chimeric antigen receptor of any of claims 17-64.

66. A vector comprising the polynucleotide of claim 65.

67. The vector of claim 66, wherein the vector further comprises a nucleic acid encoding an anti-PD-1 antibody or antigen binding fragment thereof.

68. The vector of claim 66, wherein the vector further comprises a nucleic acid encoding an anti-PD-L1 antibody or antigen binding fragment thereof.

69. The vector of any one of claims 66-68, wherein the vector is a viral vector.

70. The vector of claim 69, wherein the viral vector is a retroviral vector or a lentiviral vector.

71. An engineered cell, comprising the chimeric antigen receptor of any one of claims 17-64.

72. An engineered cell, comprising the polynucleotide of claim 65 or the vector of any one of claims 66-70.

73. The engineered cell of claim 71 or 72, wherein the engineered cell is a primary cell obtained from a subject (e.g., a human subject).

74. The engineered cell of claim 71 or 72, wherein the engineered cell is a cell line.

75. The engineered cell of any one of claims 71-74, wherein the engineered cell is an immune cell.

76. The engineered cell of claim 75, wherein the immune cell is an NK cell or a T cell.

77. The engineered cell of any one of claims 71-76, wherein the engineered cell is a T cell.

78. The engineered cell of claim 77, wherein the T cell is CD8+.

79. The engineered cell of claim 77, wherein the T cell is CD4+.

80. The engineered cell of any one of claims 76-79, wherein the T cell is isolated from a human subject.

81. The engineered cell of any one of claims 71-80, wherein the engineered cell expresses the chimeric antigen receptor.

82. The engineered cell of any one of claims 71-81, wherein the engineered cell expresses a cytokine and/or a co-stimulatory ligand.

83. The engineered cell of claim 82, wherein the cytokine and/or the co-stimulatory ligand is membrane tethered.

84. The engineered cell of claim 82, wherein the cytokine and/or the co-stimulatory ligand is secreted.

85. The engineered cell of any one of claims 82-84, wherein the cytokine is IL-2, IL-5, or IL-12.

86. The engineered cell of any one of claims 82-84, wherein the co-stimulatory ligand is CD4OL (CD154) or 41-BBL (CD137L).

87. The engineered cell of any one of claims 71-86, wherein the engineered cell expresses an antibody or antigen-binding fragment thereof (e.g., an scFv).

88. The engineered cell of any one of claims 87, wherein the antibody or antigen-binding fragment thereof is an immune checkpoint inhibitor.

89. The engineered cell of claim 88, wherein the antibody or antigen-binding fragment thereof specifically binds to PD-1, PD-L1, or CTLA-4.

90. A method for producing the engineered cell, comprising introducing a vector of claims 66-70 into a cell in vitro or ex vivo.

91. The method of claim 90, wherein the vector is a viral vector and the introducing is carried out by transduction.

92. A method of generating a population of cells, comprising introducing a nucleic acid into a cell, where the nucleic acid comprises the polynucleotide of claim 65, or a nucleic acid encoding the chimeric antigen receptor of any one of claims 17-64.

93. A method of treating an ALPP-associated disease or disorder in a subject, comprising administering the engineered cell of any of claims 71-89 to the subject.

94. The method of claim 93, wherein the ALPP-associated disease or disorder is a cancer.

95. The method of claim 94, wherein the cancer is testicular cancer, endometrial cancer, ovarian cancer, cervical cancer, urothelial cancer, pancreatic cancer, liver cancer, or stomach cancer.

96. The method of any one of claims 93-95, wherein the method further comprises administering a checkpoint inhibitor to the subject.

97. The method of claim 96, wherein the checkpoint inhibitor is an anti-PD-1 antibody or antigen binding fragment thereof, an anti-PD-L1 antibody or antigen binding fragment thereof, or an anti-CTLA-4 antibody or antigen-binding fragment thereof.

98. An anti-ALPP antibody or antigen-binding fragment thereof comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, and 3 amino acid sequences are set forth in SEQ ID NOs: 51, 52, and 53, respectively, and the selected VL CDRs 1, 2, and 3 amino acid sequences are set forth in SEQ ID NOs: 54, 55, and 56, respectively;
(2) the selected VH CDRs 1, 2, and 3 amino acid sequences are set forth in SEQ ID NOs: 57, 58, and 59, respectively, and the selected VL CDRs 1, 2, and 3 amino acid sequences are set forth in SEQ ID NOs: 60, 61, and 62, respectively;
(3) the selected VH CDRs 1, 2, and 3 amino acid sequences are set forth in SEQ ID NOs: 63, 64, and 65, respectively, and the selected VL CDRs 1, 2, and 3 amino acid sequences are set forth in SEQ ID NOs: 66, 67, and 68, respectively; and
(4) the selected VH CDRs 1, 2, and 3 amino acid sequences are set forth in SEQ ID NOs: 81, 82, and 83, respectively, and the selected VL CDRs 1, 2, and 3 amino acid sequences are set forth in SEQ ID NOs: 84, 85, and 86, respectively.

99. An antibody or antigen-binding fragment thereof that binds to ALPP comprising a heavy chain variable region (VH) comprising an amino acid sequence that is at least 90%, 91%, 92%, 93%, 94%, 95%, 96%, 97%, 98%, 99%, or 100% identical to a selected VH sequence, and a light chain variable region (VL) comprising an amino acid sequence that is at least 90%, 91%, 92%, 93%, 94%, 95%, 96%, 97%, 98%, 99%, or 100% identical to a selected VL sequence, wherein the selected VH sequence is selected from SEQ ID NOs: 1, 3, 5, 7, 9, 11, and 13, and the selected VL sequence is selected from SEQ ID NOs: 2, 4, 6, 8, 10, 12, 14, or 98.

100. The antibody or antigen-binding fragment thereof of claim 98 or 99, wherein the antibody or antigen-binding fragment specifically binds to human ALPP.

101. The antibody or antigen-binding fragment thereof of any one of claims 98-100, wherein the antibody or antigen-binding fragment is a humanized antibody or antigen-binding fragment thereof.

102. The antibody or antigen-binding fragment thereof of any one of claims 98-101, wherein the antibody or antigen-binding fragment is a single-chain variable fragment (scFv).

103. An antibody or antigen-binding fragment thereof comprising the VH CDRs 1, 2, and 3, and the VL CDRs 1, 2, and 3 of the antibody or antigen-binding fragment thereof of any one of claims 98-102.

104. A chimeric antigen receptor comprising the VH CDRs 1, 2, and 3, and the VL CDRs 1, 2, and 3 of the antibody or antigen-binding fragment thereof of any one of claims 98-103.
